(19) United States
(12) Patent Application Publication
(10) Pub. No.: US 2010/0324611 A1
(43) Pub. Date: Dec. 23, 2010

(54) DEVICES, SYSTEMS AND METHODS FOR PREVENTING AND TREATING SENSATION LOSS

(75) Inventors: Laura Deming, Lunenburg, MA (US); Marc Gibeley, Boxford, MA (US); Donald King, Lancaster, MA (US); Michael Ouradnik, Wayland, MA (US); Anthony Tremaglio, Waban, MA (US); Thomas Varricchione, Lakeville, MA (US); Bryan R. Hotaling, Harvard, MA (US); James Varney, Maynard, MA (US)

Correspondence Address: LANDO & ANASTASI, LLP, ONE MAIN STREET, SUITE 1100, CAMBRIDGE, MA, 02142, US

(73) Assignee: WAVERX, INC., Waltham, MA (US)

(21) Appl. No.: 12/635,343

(22) Filed: Dec. 10, 2009

Related U.S. Application Data
(60) Provisional application No. 61/121,469, filed on Dec. 10, 2008.

(51) Int. Cl. A61N 1/00; A47K 3/022

(52) U.S. Cl. 607/2; 4/622

(57) ABSTRACT

Certain features described herein are directed to treatment devices and treatment methods to treat and/or prevent sensation loss. The devices may take many forms including, but not limited to, a footbath, sock, slipper, sandal, insole and the like. Many different types of current and waveforms may be used to effectuate treatment.

### PRIORITY APPLICATION

This application claims priority to U.S. Provisional Application No. 61/121,469 filed on Dec. 10, 2008, the entire disclosure of which is hereby incorporated herein by reference for all purposes.

### TECHNOLOGICAL FIELD

Certain embodiments disclosed herein relate to devices, systems and methods for treating sensation loss and/or preventing sensation loss.

### BACKGROUND

Many diabetic and elderly patients have reduced or no sensation in their feet. Such reduced sensation can make it difficult or impossible to recognize the presence of injuries on the foot that could lead to skin breakdown, and development of ulcers on the foot. Infection of these ulcers can lead to amputation of the foot and/or leg.

### SUMMARY

In a first aspect, a footbath comprising a housing, a first and a second receptacle in the housing, each of the first and second receptacles sized and arranged to receive a compartment and comprising an electrical connector designed to electrically couple the compartment to the receptacle, and a power source electrically coupled to the first and second receptacles, the power source configured to provide an effective amount of current to the compartment to prevent or treat loss of sensation is described.

In certain embodiments, the footbath can include a port configured to provide fluidic coupling between the compartment in the receptacle and a container inserted into the port. In other embodiments, the container can be configured as a fluid cartridge. In some embodiments, the fluid cartridge comprises a pump for pumping fluid into the compartment and for removing fluid from the compartment. In additional embodiments, each of the receptacles is angled from front to back to facilitate use by a subject. In certain examples, the power source is configured to provide a waveform with an amplitude greater than 10 Volts, e.g., 10-300 Volts. In some examples, the power source is configured to provide a pulsed current up to about 50 milliamperes. In additional examples, the power source is configured to provide a pulse width between about 5 and about 100 microseconds. In other examples, the power source can be configured to provide a pulse spacing from leading edge to leading edge of about 100 to 250 microseconds. In additional examples, the power source can be configured to provide a pulse pair frequency between about 100 and about 200 Hertz. In certain embodiments, the power source can be configured to provide a square pulse. In additional embodiments, the power source can be configured to provide a monophasic waveform. In other embodiments, the power source can be configured to provide a biphasic waveform.

In certain embodiments, the power source can be integrated into the housing and the housing comprises an extendable arm configured to electrically couple to a remote designed to receive user input. In some embodiments, the footbath can include a remote coupled to the housing and designed to receive user input. In other embodiments, each of the receptacles is independently movable to adjust the spacing between the receptacles. In certain examples, the footbath can include a controller integrated into the housing and configured to receive user input. In additional examples, the housing comprises a retaining mechanism configured to engage the compartment to retain the compartment in the receptacle. In some examples, each of the receptacles includes an electrical coupling configured to receive an interconnect to connect a remote with the receptacle. In certain examples, the footbath comprises a cover. In additional examples, the housing comprises one or more wheels to facilitate movement of the footbath. In some examples, the housing comprises a handle. In other examples, the housing comprises a medial hinge that is configured to permit folding of the footbath. In certain examples, the footbath comprises a first electrode and a second electrode in each of the receptacles, the first electrode and the second electrode each configured to be electrically coupled to the compartment when the compartment is inserted into the receptacle. In some examples, the position of each of the first electrode and the second electrode is independently adjustable in each of the receptacles. In additional examples, the first electrode is positioned at the front of the housing and the second electrode is positioned at the back of the housing. In other examples, each receptacle further comprises a positioning mechanism to position the compartment in the receptacle. In additional examples, the footbath comprises a wireless transmitter/receiver configured to receive a signal from a remote. In other examples, the footbath comprises a docking port configured to receive a wireless remote.

In another aspect, a footbath comprising a fluid reservoir, a pair of electrodes in the fluid reservoir, and a power source coupled to the pair of electrodes and configured to provide an effective amount of current to a foot for an effective duration to prevent or treat loss of sensation of the foot is provided.

In certain embodiments, the footbath comprises an interface for receiving a fluid cartridge. In additional embodiments, the fluid cartridge comprises a pump for pumping fluid into the fluid reservoir and for removing fluid from the fluid reservoir. In other embodiments, a lower surface of the footbath is angled to facilitate use by a subject. In certain examples, the power source can be configured to provide a waveform having an amplitude greater than 10 Volts, e.g., 10-150 or 10-300 Volts. In some examples, the power source can be configured to provide a pulsed current up to about 50 milliamperes. In additional examples, the power source can be configured to provide a pulse width between about 5 and about 100 microseconds. In other examples, the power source can be configured to provide a pulse spacing from leading edge to leading edge of about 100 to 250 microseconds. In additional embodiments, the power source can be configured to provide a pulse pair frequency between about 100 and about 200 Hertz. In other embodiments, the power source can be configured to provide a square pulse. In certain examples, the power source can be configured to provide a monophasic waveform. In additional examples, the power source can be configured to provide a biphasic waveform. In some examples, the electrodes can be configured for use with a sponge comprising a fluid, the sponge configured to surround the toes of the foot. In additional examples, the footbath comprises a circuit configured to provide the effective amount of current for the effective duration. In other examples, the pair of electrodes are integrated into the fluid reservoir. In certain embodiments, the fluid reservoir comprises one or more slots for receiving and positioning the electrodes in the fluid reservoir. In additional embodiments, at least one electrode of the pair of electrodes is present as an electrode array. In some embodiments, the footbath comprises a conductive area in the fluid reservoir that is configured to receive an insole.

In an additional aspect, a compartment sized and arranged to receive a human foot and comprising a housing, an electrical connector on the housing that is configured to electrically couple the compartment to a receptacle of a footbath, and a pair of electrodes in the housing is provided.

In certain embodiments, the compartment can include a heel adjuster operative as a first electrode of the pair of electrodes and configured to be placed in contact with a heel of the human foot. In other embodiments, the compartment can include a conductive area adjacent to a toe portion of the compartment, the conductive area operative as a second electrode of the pair of electrodes. In additional embodiments, the compartment can include a cover effective to provide a substantially fluid tight seal when in a closed position. In certain examples, the compartment can include at least one port configured to receiver a container. In other examples, the port comprises a mechanism effective to puncture the container causing release of the container contents into the compartment. In some examples, the surface of the compartment comprises a conductive material in areas that contact the human foot. In additional examples, the compartment can include an insole in contact with the surface, the insole comprising a non-conductive substrate and removable areas effective to provide an electrical path between the conductive material and the foot at the removable areas. In certain examples, the compartment can include an insole in contact with the surface, the insole comprising a conductive substrate and removable areas effective to act as insulators when present in the insole. In other examples, a depth of the compartment at a toe portion of the compartment is less than a doeth of the compartment at a heel portion of the compartment to provide an angled compartment.

In another aspect, a kit comprising a footbath comprising a housing, at least one receptacle in the housing that is sized and arranged to receive a compartment, the receptacle comprising an electrical connector designed to electrically couple the compartment to the receptacle, and a compartment comprising a connector configured to couple to the electrical connector of the receptacle to provide an electrical path between a pair of electrodes in the compartment and the receptacle is disclosed.

In certain embodiments, the kit can include a power source configured to be electrically coupled to the receptacle, the power source further configured to be electrically coupled to a circuit in the footbath that is operative to provide an effective amount of current to a foot in the compartment for an effective duration to prevent or treat loss of sensation of the foot. In additional examples, the kit can include a container, and the compartment further comprises a port configured to provide fluidic coupling between the compartment and the container when inserted into the port. In other examples, the container can be configured as a fluid cartridge. In some embodiments, the receptacle is angled from front to back to facilitate use by a subject. In additional embodiments, the power source can be configured to provide an effective amount of current having an amplitude greater than 10 Volts to prevent or treat loss of sensation of the foot. In other embodiments, the power source can be configured to provide an effective amount of current having an amplitude of 10-150 Volts to prevent or treat loss of sensation of the foot. In certain examples, the power source can be configured to provide an effective amount of current having an amplitude of 10-300 Volts to prevent or treat loss of sensation of the foot. In additional examples, the power source can be configured to provide an effective amount of pulsed up to about 50 milliamperes to prevent or treat loss of sensation of the foot. In other examples, the pulsed current has a pulse width between about 5 and about 100 microseconds. In additional examples, the pulsed current has a pulse spacing from leading edge to leading edge of about 100 to 250 microseconds. In some examples, the pulsed current has a pulse pair frequency between about 100 and about 200 Hertz. In certain embodiments, the pulsed current comprises a square pulse. In additional embodiments, the power source can be configured to provide a monophasic waveform to prevent or treat loss of sensation of the foot. In other embodiments, the power source can be configured to provide a biphasic waveform to prevent or treat loss of sensation of the foot. In some embodiments, the power source can be integrated into the housing and the housing comprises an extendable arm configured to electrically couple to a remote designed to receive user input. In certain examples, the kit can include a remote coupled to the housing and configured to receive user input. In some examples, the kit can include two receptacles each of which is independently movable to adjust the spacing between the receptacles. In other examples, the housing comprises a retaining mechanism configured to engage the compartment to retain the compartment in the receptacle. In additional examples, the kit can include a first electrode and a second electrode in each of the receptacles, the first electrode and the second electrode each configured to be electrically coupled to the compartment when the compartment is inserted into the receptacle.

In another aspect, a shoe comprising a pair of electrodes and an interface for coupling the pair of electrodes to a power source configured to provide an effective amount of current to a foot for an effective duration to prevent or treat loss of sensation of the foot is disclosed.

In certain examples, the shoe includes a plurality of apertures that permit fluid entry into the shoe. In other examples, the shoe includes an on-board fluid reservoir for providing fluid to an area of a foot in the shoe. In additional examples, the pair of electrodes are positioned on an insole of the shoe. In certain examples, the shoe can be configured for use with a sponge designed to surround the toe portion of the foot. In other examples, a first electrode is adjacent to a toe portion of the shoe and a second electrode is adjacent to a heel portion of the shoe. In some examples, the effective amount of current to treat loss of sensation of the foot is administered using a waveform with an amplitude of greater than 10 Volts, e.g., 10-150 Volts or 10-300 Volts. In additional examples, the effective amount of current is a pulsed current up to about 50 milliamperes. In some examples, the pulsed current has a pulse width between about 5 and about 100 microseconds. In other examples, the pulsed current has a pulse spacing from leading edge to leading edge of about 100 to 250 microseconds. In certain examples, the pulsed current has a pulse pair frequency between about 100 and about 200 Hertz. In other examples, the pulsed current is a square pulse. In certain embodiments, the effective amount of current is provided as a monophasic waveform. In additional embodiments, the effective amount of current is provided as a biphasic waveform. In other embodiments, the shoe comprises a conductive lower surface configured to provide electrical coupling between the shoe and a receptacle of a footbath. In certain examples, the shoe comprises an adjustable heel strap that is operative as one electrode of the electrode pair. In additional examples, the shoe comprises an insole. In other examples, the insole comprises at least one removable area operative to provide an electrical path between the shoe and the foot when the removable area is removed.

In an additional aspect, a slipper comprising a fluid to be delivered to a foot and a pair of electrodes configured to be in contact with the foot, and an interface for coupling the pair of electrodes to a power source configured to provide an effective amount of current to a foot to prevent or treat loss of sensation of the foot is described.

In certain embodiments, the electrodes are positioned on a surface of the slipper that contact the bottom of a foot. In other embodiments, the slipper can include an on-board fluid reservoir for providing fluid to an area of a foot in the slipper. In additional embodiments, the power source is on-board the slipper. In some embodiments, the effective amount of current to prevent or treat loss of sensation of the foot is administered using a voltage of 10-300 Volts. In additional embodiments, the effective amount of current is provided as a pulsed current up to about 50 milliamperes. In other examples, the pulsed current has a pulse width between about 5 and about 100 microseconds, a pulse spacing from leading edge to leading edge of about 100 to 250 microseconds, and a pulse pair frequency between about 100 and about 200 Hertz. In some examples, the pulsed current is a square pulse. In certain examples, the effective amount of current is provided as a monophasic waveform or a biphasic waveform.

In another aspect, a sock comprising a pair of electrodes and an interface for coupling the pair of electrodes to a power source configured to provide an effective amount of current to a foot to prevent or treat loss of sensation of a foot is provided.

In certain embodiments, the sock can include a carrier comprising a fluid. In some embodiments, the sock can include an on-board power source. In additional embodiments, the electrodes are positioned on a lower surface of the sock that contacts the bottom of the feet. In other embodiments, the effective amount of current to prevent or treat loss of sensation of the foot is administered using a voltage of 10-300 Volts. In certain examples, the effective amount of current is provided as a pulsed current up to about 50 milliamperes. In additional examples, the pulsed current has a pulse width between about 5 and about 100 microseconds, a pulse spacing from leading edge to leading edge of about 100 to 250 microseconds, and a pulse pair frequency between about 100 and about 200 Hertz. In other examples, the pulsed current is a square pulse. In some examples, the effective amount of current is provided as a monophasic waveform or a biphasic waveform.

In additional aspect, an electrical bandage comprising a porous sleeve configured to retain a fluid, and a pair of electrodes in or coupled to the porous sleeve and configured to couple to a power source to provide an effective amount of current to a foot to prevent or treat loss of sensation of the foot is disclosed.

In certain embodiments, the bandage can include a conductive material in the sleeve. In other embodiments, the bandage can include an on-board power source. In additional embodiments, the bandage can include a scrim configured to retain fluid within the electrical bandage. In some embodiments, the power source can be configured to provide the effective amount of current using a voltage of 10-300 Volts to prevent or treat loss of sensation of the foot. In other embodiments, the power source is configured to provide the effective amount of pulsed current up to about 50 milliamperes to prevent or treat loss of sensation of the foot. In certain examples, the power source can be configured to provide the pulsed current having a pulse width between about 5 and about 100 microseconds, a pulse spacing from leading edge to leading edge of about 100 to 250 microseconds, and a pulse pair frequency between about 100 and about 200 Hertz. In additional examples, the pulsed current is a square pulse. In other examples, the power source is configured to provide the effective amount of current as a monophasic waveform or a biphasic waveform to prevent or treat loss of sensation of the foot.

In another aspect, a method comprising identifying and/or selecting a subject having loss of sensation, and treating the loss of sensation using one or more of the devices described herein. In some examples, the treating step is performed in the absence of a drug agent.

In an additional aspect, a method comprising identifying and/or selecting a diabetic subject having reduced sensation, and preventing further sensation reduction by treating an area of the diabetic subject using one or more of the devices described herein. In certain examples, the preventing step is performed in the absence of a drug agent.

In another aspect, a method comprising identifying and/or selecting a subject having loss of sensation in a foot, and administering an effective amount of current to the foot to treat the sensation loss using a waveform with an amplitude of 10-300 Volts, a pulsed current up to about 50 milliamperes, a pulse width between about 5 and about 100 microseconds, pulse spacing (from leading edge to leading edge) of about 100 to 250 microseconds, a pulse pair frequency between about 100 and about 200 Hertz, a square pulse, and a monophasic or a biphasic waveform is described. In certain examples, the administering step is performed in the absence of a drug agent.

In another aspect, a method comprising identifying and/or selecting a diabetic subject having reduced sensation in a foot, and administering an effective amount of current to the foot to prevent further sensation loss using a waveform with an amplitude of 10-300 Volts, a pulsed current up to about 50 milliamperes, a pulse width between about 5 and about 100 microseconds, pulse spacing (from leading edge to leading edge) of about 100 to 250 microseconds, a pulse pair frequency between about 100 and about 200 Hertz, a square pulse, and a monophasic or a biphasic waveform is disclosed. In some examples, the administering step is performed in the absence of a drug agent.

Additional features, aspect, examples and embodiments are described in more detail below.

### BRIEF DESCRIPTION OF THE FIGURES

Certain embodiments are described with reference to the figures in which:

FIG. 1 is a side-view illustration of a treatment device, in accordance with certain examples;

FIGS. 2A-2D are illustrations of a treatment device having separate compartments and

FIG. 2E is an illustration of a remote suitable for use with the treatment device shown in FIGS. 2A-2D, in accordance with certain examples;

FIG. 3 is an illustration of a treatment device having separate compartments and a wired remote, in accordance with certain examples;

FIGS. 4A and 4B are illustrations of a treatment device having separate compartments with handles and a wired remote, in accordance with certain examples;

FIGS. 5A and 5B are illustrations of a treatment device having a remote that can detach to an extendable arm, in accordance with certain examples;

FIGS. 6A and 6B are other illustrations of a treatment device having a remote that can detach to an extendable arm, in accordance with certain examples;

FIG. 7 is an illustration of a treatment device that includes a cover, in accordance with certain examples;

FIG. 8 is an illustration of a treatment device with a compartment that includes a cover, in accordance with certain examples;

FIGS. 9A-9D are illustrations of a treatment device that includes a port configured to receive a container, in accordance with certain examples;

FIGS. 10A-13B are illustrations of treatment devices that include portability features, in accordance with certain examples;

FIG. 14 is an illustration of a shoe or boot, in accordance with certain examples;

FIG. 15 is an illustration of an insole suitable for use with the treatment devices described herein, in accordance with certain examples;

FIGS. 16A-16C are illustration of a treatment device configured as a shoe and used with a sponge, in accordance with certain examples;

FIG. 17 is an illustration of a treatment device including an adjustable heel strap, in accordance with certain examples;

FIG. 18 is an illustration of a bathtub, in accordance with certain examples,

FIG. 19 is an illustration of a sock, in accordance with certain examples;

FIGS. 20A-23B are schematics of devices for providing treatment, in accordance with certain examples;

FIG. 24 is an illustration of a remote for controlling a treatment device in accordance with certain examples;

FIG. 25 is an illustration of a treatment device that is used with an insole, in accordance with certain examples;

FIG. 26 is an illustration of a treatment device having receptacles whose positions can be adjusted, in accordance with certain examples;

FIGS. 27A and 27B are illustration of a treatment device, in accordance with certain examples;

FIGS. 28-37 show the results of various measurements, in accordance with certain examples;

FIG. 38 is a circuit diagram of a pulse generator, in accordance with certain examples;

FIGS. 39A-39F is a block diagram of a controller (FIG. 39A) and associated circuit diagrams (FIGS. 39B-39F) and FIGS. 39G-39N show illustrative waveforms, pulses and pulse trains that can be generated using the controller;

FIG. 40 is a graph showing the relationship between the coefficient of variation and the spacing of the heel electrode away from the foot, in accordance with certain examples;

FIG. 41 is a graph showing the relationship between the coefficient of variation and the conductivity of the treatment solution, in accordance with certain examples;

FIGS. 42A and 42B show a treatment device that includes a bridging controller, in accordance with certain examples;

FIGS. 43A-43D show a treatment device for use with a mat that is configured to position the treatment device, in accordance with certain examples;

FIG. 44 shows a treatment device including a cover configured to receive a controller, in accordance with certain examples; and

FIG. 45 shows a satchel configured to receive a treatment device, in accordance with certain examples.

It will be recognized by the person of ordinary skill in the art, given the benefit of this disclosure, that certain dimensions or features in the figures may have been enlarged, distorted or shown in an otherwise unconventional or non-proportional manner to provide a more user friendly version of the figures. Where dimensions are specified in the description below, the dimensions are provided for illustrative purposes only.

### DETAILED DESCRIPTION

In certain examples, the devices, systems and methods described herein may be used to treat and/or prevent sensation loss. As used herein, “sensation loss” generally refers to reduced ability to sense stimulus or pain anywhere including the hands, legs, feet, etc. “Loss of protective sensation” is a subset of sensation loss and refers to the inability to sense stimulus or pain specifically in the feet. For ease of description, the devices, systems and methods are described in certain instances in connection with preventing and/or treating sensation loss with certain devices constructed and arranged particularly for treatment of loss of protective sensation. The devices may be adapted or constructed and arranged, however, to prevent and/or treat sensation loss anywhere on the body including but not limited to, the fingers, hands, arms, legs, feet, toes or other body areas or portions thereof. Subjects can be identified as having loss of sensation according to one or more well known protocols with illustrative protocols described below.

Certain devices are described herein as “treatment devices.” This term is intended to include many different configurations that can provide an electric current, voltage or the like to treat and/or prevent loss of sensation. Illustrative types of treatment devices are described in more detail below. The treatment devices and methods described herein are effective to treat or prevent loss of protective sensation and other disorders in the absence of a drug agent. In particular, the devices and methods described herein can be effective to treat or prevent loss of sensation even though they are not functioning as iontophoresis or electrophoresis devices. For example, the current parameters and waveforms themselves, when used in combination with one or more of the treatment devices, are effective to treat many different disorders including loss of protective sensation.

In certain embodiments, the devices and methods described herein may be used on many different subjects. For example, diabetics with severe ischemic ulcers in feet that are destined for amputation may particularly benefit from using the devices and methods disclosed herein. These diabetics may also have underlying osteomeyelitis, which does not respond well to standard therapy including systemic antibiotics and wound care. The instant methods and devices can be used to greatly improve the management of these and other conditions.

In certain examples, the devices and methods disclosed herein can be used to stimulate, at least in part, the activity of fibroblasts in the healing process. For example, in the healing of ischemic ulcers the fibroblasts act first to build the framework upon which further cell types including skin and capillaries grow. In some embodiments, the devices and methods can stimulate activity in bone cells as well, which accelerates fracture healing. It may be desirable to alter the particular amount of current, voltage, etc., that is provided by the devices described herein to promote one or more of fibroblast activity, bone cell growth or the like. In certain embodiments, such methods can be provided simultaneously with the treatment methods described herein or in addition to the treatment methods described herein.

In other examples, the devices and methods described herein can be used in preventing and/or reversing neuropathy in the feet and legs of diabetics (or others) being treated. The term “neuropathy” refers generally to disorders of the nervous system and, in particular, diseases or conditions that affect the nerves or nerve cells. There are many different types of neuropathy including, but not limited to, peripheral neuropathy, cranial neuropathy and other neuropathies that can cause neuropathic pain. Peripheral neuropathy refers to conditions of the peripheral nerves, e.g., those nerves outside of the brain and spinal cord. Cranial neuropathy refers to conditions of the cranial nerves, e.g., those nerves extending directly from the brain stem, and may be subdivided into conditions affecting individual cranial nerves, e.g., the optic nerve, the auditory nerve, etc. Other types of neuropathies also exist including diabetic neuropathy, polyneuropathy, neuropathic arthropathy, autonomic neuropathy, compression mononeuropathy and others. Neuropathy is diagnosed or identified in many instances after the appearance of symptoms. The particular symptoms that appear depend on the neuropathic condition and may include numbness, dizziness, muscle weakness, muscle contractions, burning pain, and dysesthesia. The devices and methods described herein are particularly suitable for treatment of neuropathic conditions in the limbs and appendages, but they may be adapted to treat other types of neuropathic conditions in other areas of the body.

In certain embodiments, other illustrative treatable conditions using the methods and devices provided herein include, but are not limited to, the following: (1) diabetic ulcers or ischemic ulcers in the bedfast or neurologically compromised patient are characterized by decreased healing due to ischemia and compromise in the microcirculation, which the present treatment can enhance and remodel; (2) large decubiti requiring surgical closure with skin, fat or muscle flaps can benefit from preheating before the closure by improved blood flow, granulation, and epithelialization of wound margins; (3) sports injuries including sprains and strains can benefit from more rapid healing due to the enhanced blood flow, the increased activity of the fibroblasts and the reeducation of the entire muscle mass as well as the ligaments and tendons; (4) repetitive stress injuries such as carpal tunnel syndrome characterized by an imbalance between the wear and tear in the tissues and an inadequate healing and repair response usually respond rapidly to the present treatment; (5) chronic pain syndromes such as fibromyalgia and chronic low back pain benefit from a decrease in pain and an increase in flexibility and function; (6) healing time for bone fractures can be decreased due to the increased blood flow as well as the direct stimulation of the bone by the methods and devices described herein; (7) ischemic rest pain conditions due to arterial insufficiency can be improved with the methods and devices described herein; (8) degenerative arthritic conditions including osteoarthritis and degenerative joint disease can be improved through the enhanced blood flow and healing provided by the methods and devices described herein; (9) the methods and devices described herein can prevent or reverse diabetic neuropathy and keep it reversed; and (10) the methods and devices described herein can be used to increase the effectiveness of other medical treatments as well, e.g., can be used to treat neuropathic loss of sensation secondary to chemotherapy. The devices and methods are particularly suited to treat loss of protective sensation and/or prevent loss of sensation.

### Waterbath Treatment Devices

While certain devices are described or referred to below for convenience purposes as a “waterbath” or “footbath,” the device can be used to treat disorders using fluids other than water and can treat disorders on areas other than the feet. Where the waterbath is described as having a single fluid reservoir, multiple different reservoirs may also be used, e.g., two or more reservoirs each sized and arranged to receive a single foot or other appendage such as the hand or arm. Where two or more reservoirs are used, each of the reservoirs may be separately provided with a current independent of the other reservoir, or the two reservoirs may be provided with the same level and type of current. In addition, two or more individual fluid reservoirs, which are fixed or removable from a larger housing, may be provided to facilitate use of the fluid reservoirs or to permit the use of different types or sizes of fluid reservoirs with a single housing. Similarly, where reference is made to “electrodes” or “pairs of electrodes,” such reference is for convenience purposes only. The electrodes may be present in a single pair, multiple pairs, electrode arrays, or may take other forms where a plurality of electrodes are present. In some examples described below, individual components may include conductive devices or materials that when coupled together can function as an electrode or an electrode array. For example, a treatment compartment may include a conductive area that when coupled to a conductive area on a receptacle permits the conductive area of the compartment to function as an electrode.

In addition, certain embodiments are described below as including a fluid. However, by placement of the electrodes in direct contact with the area to be treated, “fluidless treatment” may be performed such that the entire area need not be immersed in a fluid. For example, the surface of the area to be treated may be wetted, and an electrode can be placed in contact with the wetted area without having to immerse the entire area in a fluid.

Existing footbath designs require a significant volume of fluid to cover the patient's feet because there is a substantial amount of space between the outer surface of the subject's feet and the inner surface of the container which is typically rectangular with an open top. The need for a significant volume of fluid creates a challenge for the patient to fill and empty the bath. To fill the bath, the patient must either fill the bath at a sink and then carry the heavy, filled bath to the preferred treatment location which will likely result in spilled bath solution or the patient must fill a separate container and carry that container to the bath in the preferred treatment location, possibly multiple times. To empty the bath, the patient must either carry the heavy filled bath back to the sink which will likely result in spilled bath solution or must empty the bath solution into a separate container and then carry that container to the sink, possibly multiple times.

In certain embodiments described herein, the waterbath can include a single fluid reservoir or may be divided into two or more individual fluid reservoirs referred to in certain instances as “compartments.” Where individual compartments are present, use of both compartments is not required. In particular, treatment of only one foot or area can be performed and the other compartment can remain unused. In some examples, a compartment can be inserted into a receptacle in the housing that is configured to mate the compartment to electrical components (or other components) of the device. Where one or more compartments are present, the compartment may include user-friendly features including, but not limited to, being separable from a base unit or housing, a mechanism to releasably retain the compartment in the base unit, can be angled downward, upward, outward or inward for better positioning of the area to be treated, can be shaped or contoured to the area to reduce the fluid volume used in the treatment methods, can include drainage features such as pumps, spouts, plugs and the like, can be formed of plastic or lightweight material to reduce the overall weight, can be configured to be independently controlled or simultaneously controlled with another compartment, can include texturing, non-slip surfaces, arch support, can include insoles, slippers, socks, sleeves, pads, spacers or sponges, can include one or more covers for spill containment and/or storage purposes, and can include electrodes or conductive areas that can couple to electrodes to facilitate treatment using one or more of the treatment methods described herein. These and other features are described in more detail below.

In certain embodiments where two or more fluid reservoirs, e.g., compartments, are present, the reservoirs may be coupled to each other through a controller which is operative to provide the treatment parameters to each of the reservoirs. The controller can act as a bridge to connect the two fluid reservoirs to each other. Examples of devices that include multiple fluid reservoirs are described in more detail below.

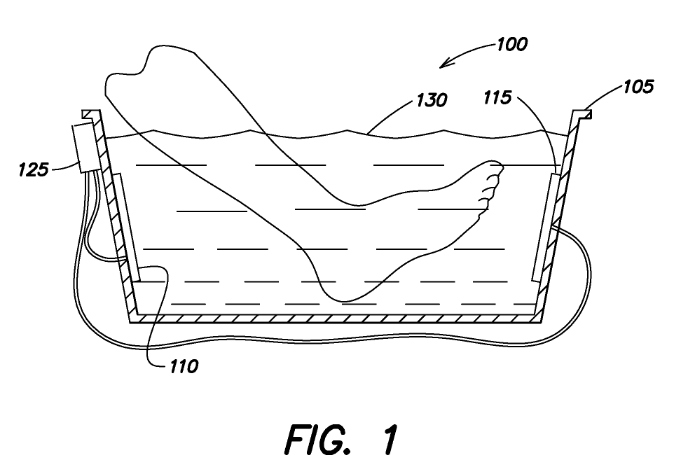

In certain examples, a waterbath may be used to treat and/or prevent sensation loss. Referring to FIG. 1, the waterbath 100 comprises a fluid reservoir 105 that may be sized and arranged to receive a specific appendage or portion of the body. For example, the waterbath may be sized and arranged to receive one or both hands, one or both feet, an entire leg or legs, the lower body portion of a subject, e.g., below the waist, or the entire body of a subject. The waterbath shown in FIG. 1 is sized and arranged to receive one or both feet but the principal design is applicable to other waterbaths. The waterbath 100 also includes a first electrode 110 and a second electrode 115 that provide current to the foot 120 in the fluid reservoir 105. The current can be provided directly to the foot 120 or may be provided through a fluid 130 disposed in the fluid reservoir 105. As discussed in more detail below in the “Waveform, Current and Controllers and Treatment Methods” section, the current may be a monophasic current, a biphasic current, a continuous current, a pulsed current or other forms of a current that can prevent and/or treat sensation loss or other disorders discussed herein. In the embodiment shown in FIG. 1, a voltage source 125 is integrated into the fluid reservoir 105 such that connection of the voltage source to a battery, standard 110V AC residential outlets or other sources provide the current for treatment. Various current sources are described in more detail below.

During treatment using the waterbath 100, a subject places one or both feet in the bath. A fluid 130 may be added to the fluid reservoir 105. The fluid may be substantially pure water, tap water, water or other conductive media including therapeutics, antimicrobials, detergents or other species as described herein. Current is applied through the electrodes 110 and 115 in an effective amount for an effective duration to treat the sensation loss or to prevent sensation loss, e.g., to prevent further sensation loss. The treatment regimen may vary as described in more detail below.

While the waterbath is shown in FIG. 1 as having a substantially flat surface from which the foot may rest, the base of the footbath may be tilted or angled to increase user comfort. In addition, one or more wedges, which may be removable or fixed, or other devices may be inserted in the bath to provide a user selected angle for overall comfort.

In certain embodiments, the housing of the waterbath can provide structural support for the fluid reservoir and can also include other features. For example, the housing can be an enclosure for electronics module and cable routing so there are no components accessible to the user or to get in the way. The lack of external cables provides for easier user setup (no connections to be made by the user or opportunity for errors in setup) and reduces safety hazards. The housing may also include integrated handles or other means of lifting and moving the housing for adjustment by the user before, during and after usage, e.g., by locating wheels on the front portion of the housing, the rear can be lifted to move the waterbath. The housing can include or be used with a floor mat to protect areas under the unit from spillage during setup, treatment or cleanup. In some examples, the housing can include feet or other features mounted to the housing to facilitate use and/or storage of the waterbath.

In certain examples, the housing can include an integrated display. In some embodiments, the display has large characters for easy readability by subjects during treatment. The display can be angled or adjusted to a desired viewing angle by the end-user. The display may be waterproof or sealed in the event fluid is spilled on the display. As described in more detail below, in certain examples, the display may be present on a remote instead of the housing or may be present on both a remote and the housing.

In some examples, a remote can be used with the waterbath. The remote can be wired or wireless and, for example, can be shaped for comfortable positioning in hand. The remote may include a backlight control to light the display in dim settings, but lower battery consumption when not backlit. The remote can be used to select a treatment method that is wirelessly transmitted to the waterbath, or can be coupled to the waterbath to transfer a selected treatment method to the waterbath. One or more storage compartments can be integrated into the housing such that the remote is not misplaced during storage. If misplaced, the waterbath can include suitable locator electronics that may be activated to assist in finding the remote, e.g., an audible sound generated by remote when called by the waterbath. Other features on a remote may also be present and some of these are described further below.

In certain examples, the fluid reservoirs described herein may include one or more “anti-spill” features including lateral legs, partially occluded top surfaces or the like to reduce the likelihood that fluid is lost during transport and/or use. In other embodiments, the fluid reservoir can be configured to receive a cover which can snap into place, be held in place with clips or may be friction fitted to the fluid reservoir such that fluid in the fluid reservoir does not spill out during movement of the waterbath.

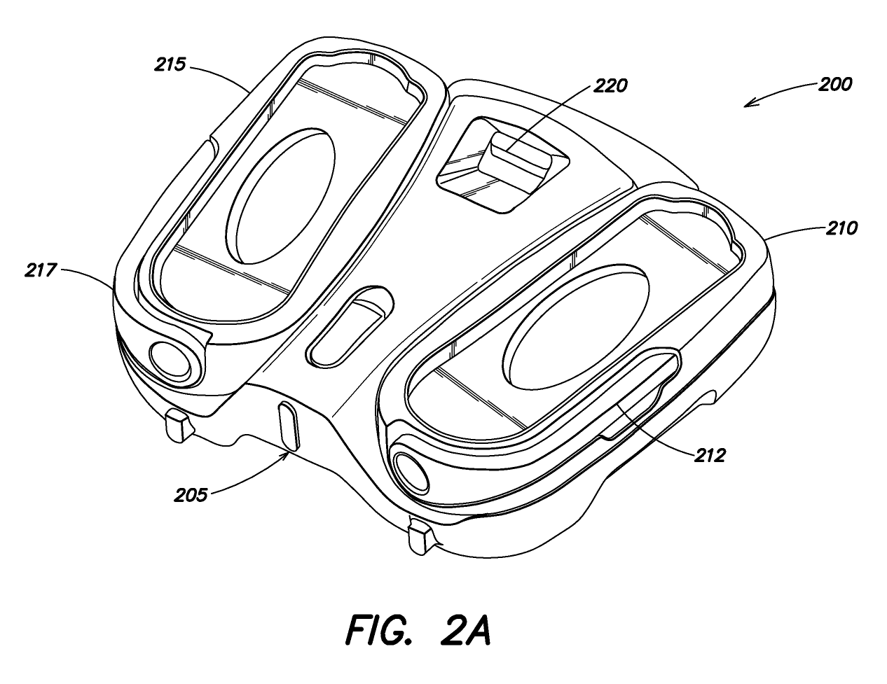

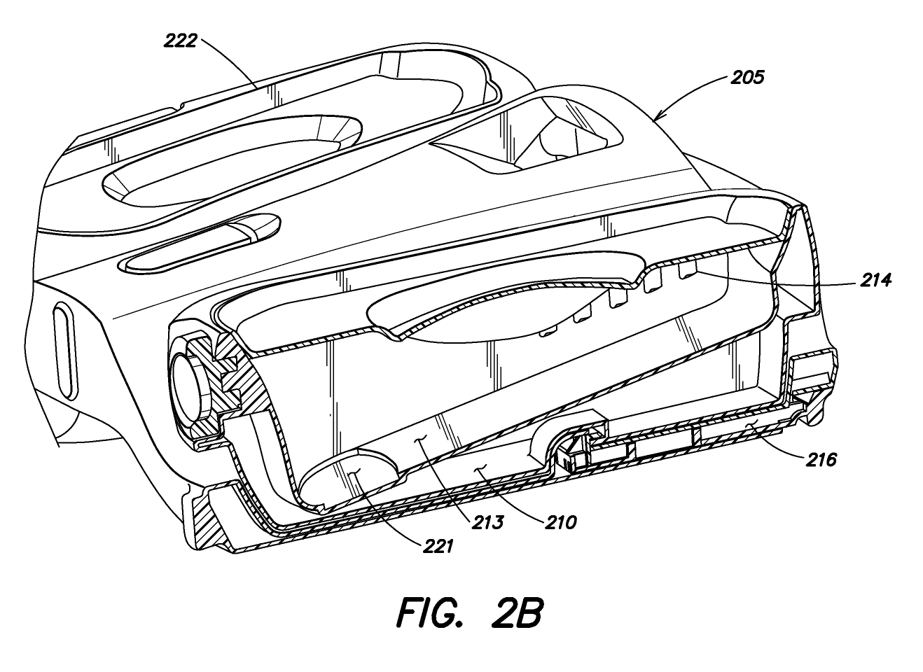

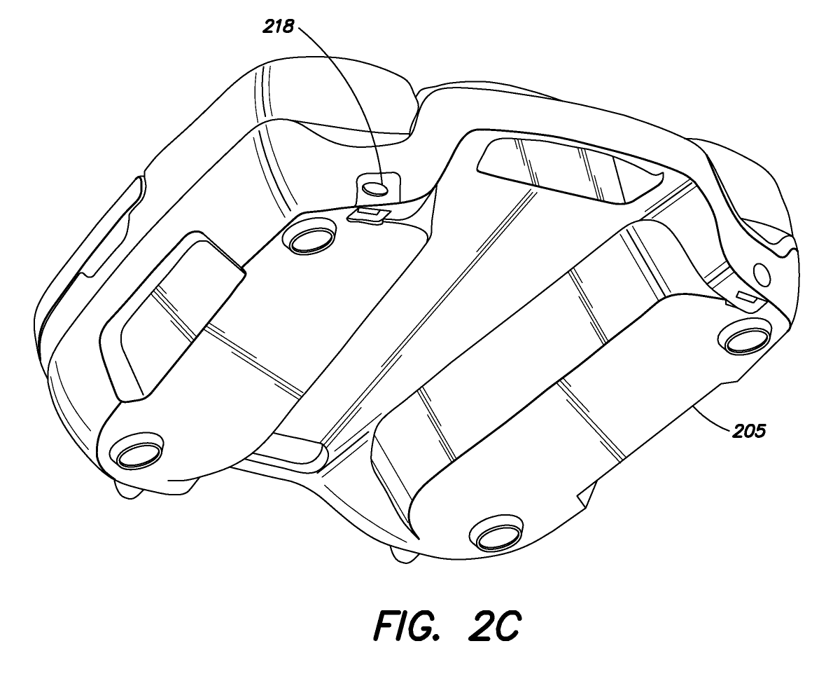

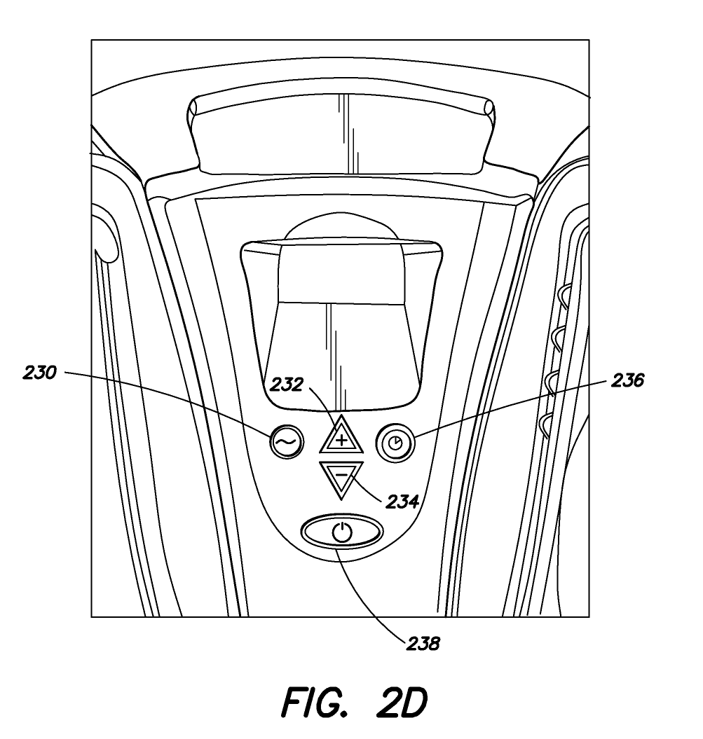

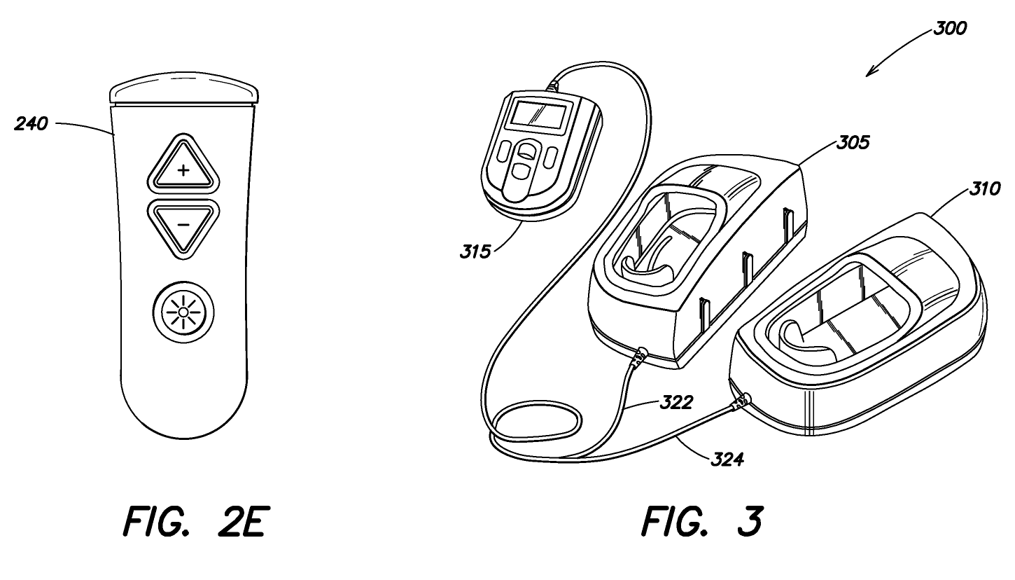

In some embodiments, the waterbath may be configured with individual compartments that can be inserted into a housing to facilitate treatment. One example of such a configuration is shown in FIGS. 2A-2F. Referring to the perspective view shown in FIG. 2A, the waterbath 200 includes a housing 205, a first compartment 210 and a second compartment 215 that are configured to be placed in or on receptacles in the housing 205 to effectuate treatment. The first compartment 210 and the second compartment 215 each can include a handle 212 and 217, respectively, to facilitate lifting of the compartments out of the housing. In particular, the handles can provide for easier disposal of a fluid in each of the compartments 210 and 215 after treatment has been ended. By separating the fluid reservoir into individual compartments several advantages are achieved including, but not limited to, the ability to use a single housing with different sized compartments, the ability for users with limited mobility from neuropathic conditions to empty lower volumes of fluid from each compartment as compared to a fluid reservoir with one large single compartment, the ability provide treatment to only a single foot or other appendage rather than both feet, the ability to place different electrodes in close proximity to affected areas on each feet, and the ability to vary the current or voltage provided to each compartment.

Referring again to FIG. 2A, the waterbath 200 also includes a display/controller 220 that can be used to display treatment parameters or can be used to program the treatment parameters into the waterbath 200. As discussed further below, the waterbath 200 may be used with an associated remote to facilitate programming of the device without having to bend over to enter the treatment parameters.

In certain examples, the compartments may be configured to receive one or more electrodes such as those described below or may have integrated electrodes or conductive areas. A cross-section of one of the receptacles is shown in FIG. 2B. The compartment 210 comprises a fluid reservoir 213, a plurality of slots such as slot 214 that can receive one or more electrodes, a depression 221 that can receive another electrode and a cover 222 that can provide a substantially fluid tight seal when the compartment 210 is being transported. The compartment 210 may also include a retaining mechanism 216 (see FIGS. 2B and 2C) that is effective to engage the housing 205 in at least some manner to retain the compartment 210 in the housing 205 for at least some period.

For example, the receptacle of the housing may include a suitable opening or catch that can engage a complementary feature on the compartment to lock the receptacle into place. Such locking into place using mechanical components is referred to herein as an “active retaining feature.” Once engaged, the catch can be released by depression of a release or area on the housing. FIG. 2C shows one such release 218, which is coupled to the retaining mechanism 216, positioned on a bottom surface of the housing 205. The exact placement of the release is not critical and in certain examples, the release can be placed on the same surface as the controls to facilitate easy depression of the release. In some examples, increased downward pressure applied to the compartment can result in release of the compartment. For example, the compartment can first be placed in the housing minimal force. Once engaged, treatment can be performed using the waterbath. The user may then stand, or otherwise push down on the compartment, using additional force, which causes release of the compartment from the housing. Illustrative retaining mechanisms include, but are not limited to, springs, spring loaded slides, release latches, hole-and-pin mechanisms, catches and other mechanical devices that are operative to mate one component to another for at least some period. In certain embodiments, the release may be actuated through one or more features on the remote control such that a user with limited mobility can disengage the compartment by pushing a button on the remote control. For example, the release mechanism may include an actuating device such as a motor that can cause the release mechanism to be moved and thereby release the compartment. Such automated release may be particularly desirable for elderly subjects with limited hand strength.

In other examples, the compartment can include one or more passive retaining features such as, for example, guides or pins on a surface that mates to the housing, with the pins designed to position the compartment at a suitable place in or on the housing. In this configuration, the compartment is retained by the housing under gravitational forces rather than any locking components. In other embodiments, a combination of passive retaining features and the active retaining features may be used. For example, one or more guides or pins may be used to facilitate placement of the compartment, and an active release mechanism may be present to securely fasten the compartment to the housing until a release is depressed or otherwise actuated.

In certain embodiments and referring to FIGS. 2D and 2E, the waterbath 200 may be operated using controls located on the housing (FIG. 2D) or using a remote control (FIG. 2E) or both. The exact number of buttons may vary depending on the treatment that the waterbath 200 is designed to accomplish. In some examples, the waterbath 200 can include a button 230 to select a particular waveform, buttons 232 and 234 to increase or decrease treatment currents, voltage parameters, etc., a button 236 to select a desired treatment time and a power button 238. In other examples, a remote 240 (FIG. 2E) can be used to program the waterbath instead of, or in addition to, the controls used on the waterbath display. In operation, a user can select the desired treatment parameters, as described further below, using the remote or the controls or both.

In certain examples, the compartments may each be coupled to a wired controller (and optionally can be coupled to each other) as shown in FIG. 3. The device 300 includes a first compartment 305 and a second compartment 310, each of which is coupled to a remote 315 through interconnects 322 and 324. The interconnects 322 and 324 can be permanently attached to the compartments 305 and 310 or can be plugged into the compartments 305 and 310 prior to operation. The compartments 305 and 310 may be coupled to a housing that includes a power source and the desired circuitry to provide treatment or the circuitry and power sources can be integrated into each of the compartments 305 and 310 such that the waterbath 300 can provide treatment without the need to use additional devices. In some embodiments, the compartments 305 and 310 can be decoupled from the remote 315 so that any fluid added to the compartments can be emptied after use.

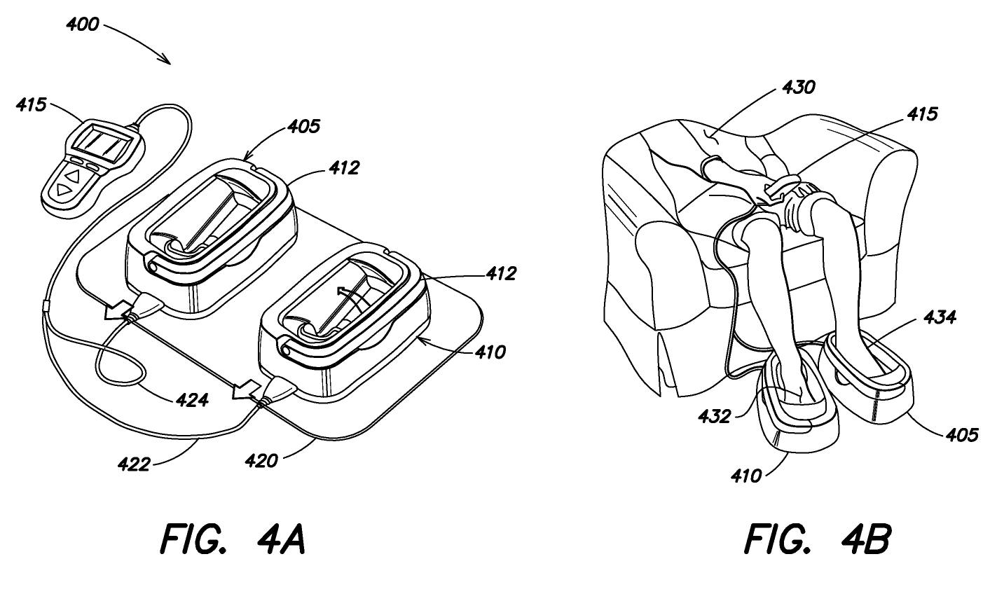

In certain embodiments, the separate compartments may each include a handle to facilitate transport and/or use of the treatment device. FIGS. 4A and 4B show another treatment device that includes separate compartments each of which has a handle. The device 400 includes compartments 405, 410 each of which has a handle, such as the handle 412 on the compartment 410. The handle 412 may be configured to rotate upward to facilitate transport of the compartment 410. As shown in FIG. 4A, the device 400 has been placed on a mat 420 to protect the underlying surface from any fluid spills, though this placement is optional. The compartments 405, 410 are electrically coupled to a remote 415 through interconnects 422 and 424. A user 430 can place one or more of their feet 432, 434 into the compartments 405, 410 as shown in FIG. 4B. The user can then select the desired treatment parameters using the remote 415. Once treatment is completed, the user 430 can then disconnect the interconnects 422 and 424 from the compartments 405 and 410 and empty each compartment separately.

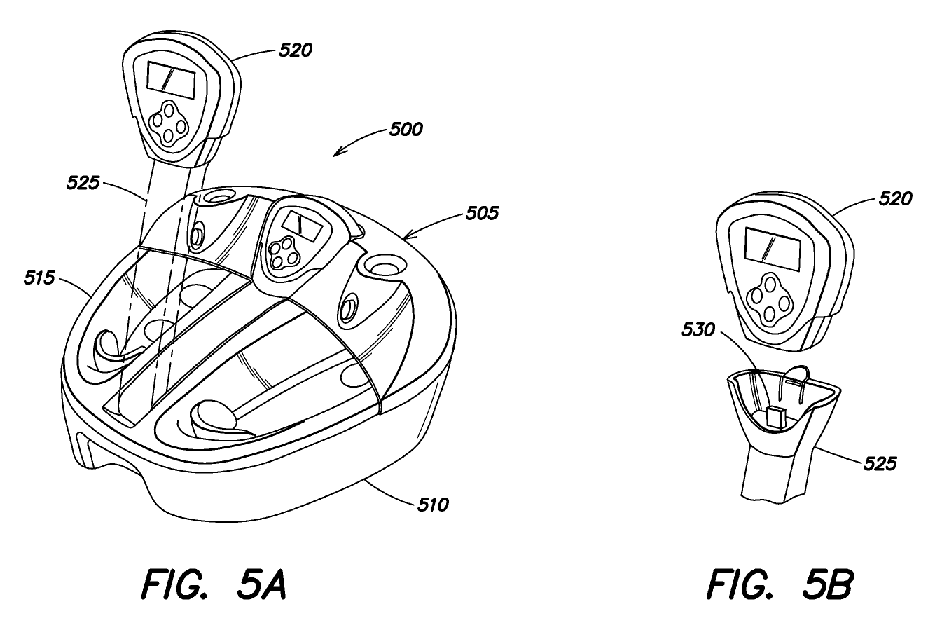

In certain examples, the separate compartments can be integrated into a waterbath and may be configured as fixed compartments. One such example is shown in FIGS. 5A and 5B. Referring to FIG. 5A, the device 500 includes a housing 505 comprising a first compartment 510 and a second compartment 515. The device 500 also includes a removable remote 520 that is coupled to the device 500 through an arm 525. While not shown, the device 500 may include a suitable power supply and circuitry to provide a desired type of treatment, as discussed in more detail below. The arm 525 of the device 500 may be depressed to a position such that it is flush with the top of the housing 505 or may be raised to a position that is substantially orthogonal to the top planar surface of the housing 505. In some examples, the arm 525 may be extended vertically to place the remote 520 at a desired height relative to the user. For example, it may be desirable for a user to have the remote 520 positioned at shoulder height when they are seated in a chair so that they can program the remote 520 more easily. In such instances, the arm can include internal sleeves that can be extended and locked into place at various positions. In addition or in the alternative, the remote 520 can be disengaged from the arm 525 to permit programming of the remote 520. Once programmed, the remote 520 can be reinserted into the arm 525 and treatment can begin. The arm 525 can include a suitable connector 530 to electrically couple the controller 520 to the other electrical components of the device 500.

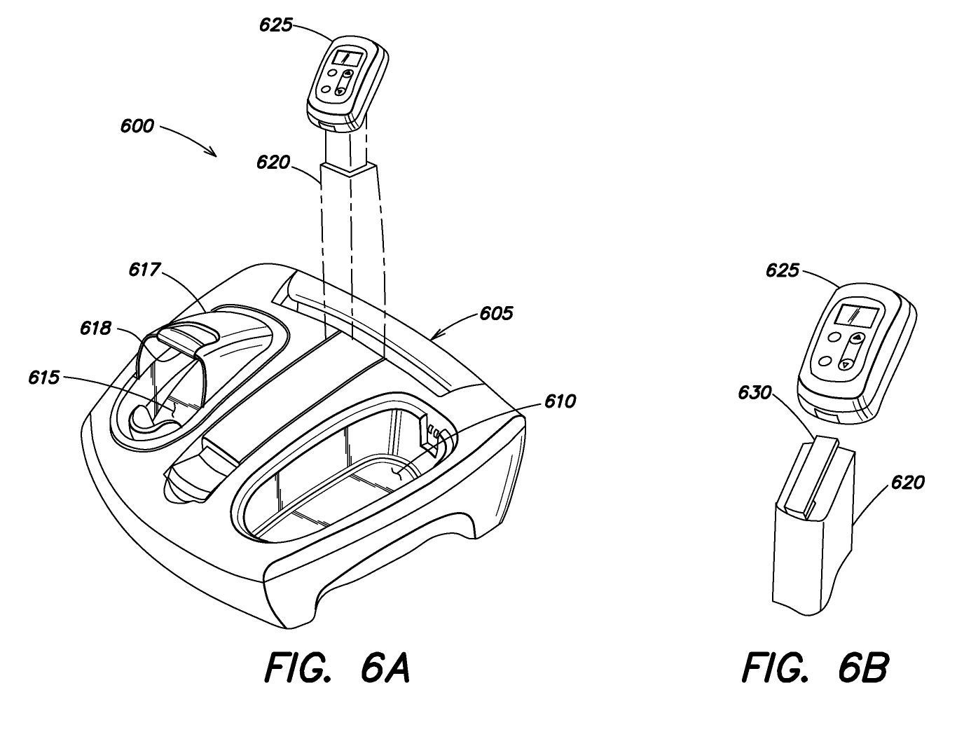

In some examples, the arm used to couple the programmer to the waterbath can be positioned at different sites on the waterbath. One example is shown in FIGS. 6A and 6B. Referring to FIG. 6A, the waterbath 600 includes a housing 605 having first and second compartments 610 and 615. The waterbath 600 also includes an extendable arm 620 positioned on a top, forward surface of the housing. The extendable arm 620 can be configured to fold toward the rear of the waterbath 600 for storage. In operation, the extendable arm 620 can be tilted up toward the front of the waterbath 600. The extendable arm 620 can then be extended upward to a desired height to facilitate use of the waterbath 600. In some embodiments, a remote 625 can be used to program the waterbath 600 for treatment. In certain examples, the remote 625 can be fixed or may be removable as shown in FIG. 6B. For example, the remote 625 can be removed such that it can be programmed by a user and then replaced on the extendable arm 620 during use. The extendable arm 620 can include a connector 630 to provide electrical coupling of the remote 625 to the waterbath 600. The waterbath 600 also is shown as including an optional cover 617 over the compartment 615. The cover 617 is shown as having a handle 618 which can facilitate placement of the footbath at a desired area or can be used to remove the compartment 615 from the housing 605 for emptying of fluid in the compartment 615.

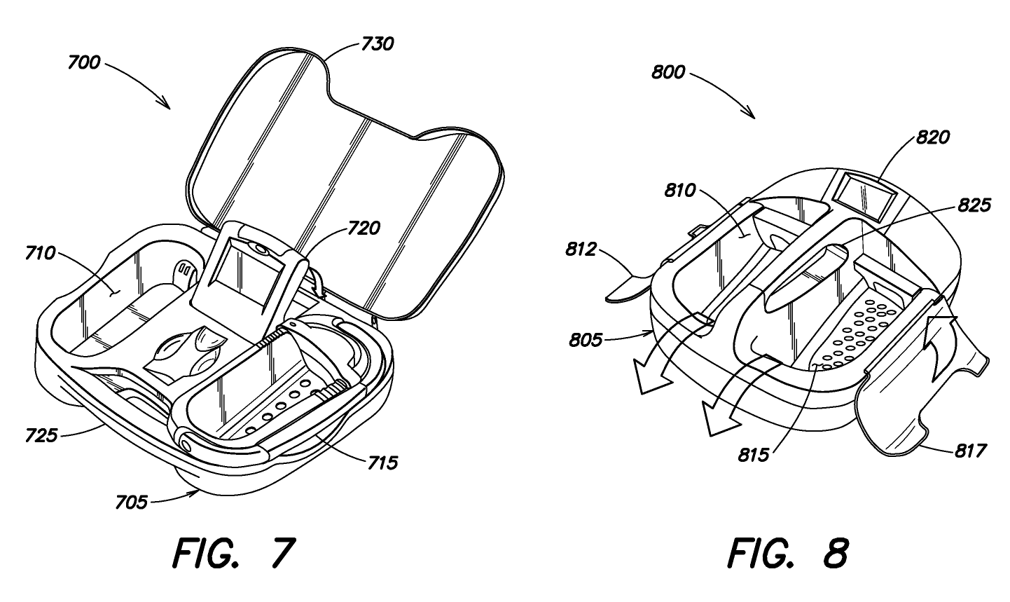

In certain examples, the waterbath suitable for use in the treatment methods described herein can include a cover that releasably engages the housing to provide a substantially fluid tight seal. Such substantially fluid tight seal permits transport of the fluid in the waterbath and can reduce contamination of the waterbath by particles such as dust or microorganisms such as fungal spores. The cover can be present in waterbaths having one fluid reservoir or waterbaths having two individual compartments. Referring to FIG. 7, a waterbath 700 comprises a housing 705, a first receptacle 710 configured to receive a compartment, such as compartment 715, a display 720, a handle 725 and a cover 730. The compartment 715 can be inserted into the receptacle on the right side of the waterbath 700 and can engage an electrical connector such that current can be provided to a fluid in the compartment 715. The compartment 715 may also include a handle, as described herein, to facilitate removal and transport of the compartment. The display 720 is configured to display and/or receive input parameters entered by a user. The handle 725 of the waterbath 700 can be used to transport the housing 705 to a desired location or to position the waterbath 700 suitably for treatment. The cover 730 of the waterbath 700 can be attached to a front surface of the housing 705 through a hinge mechanism such that the cover 730 can be raised and lowered as desired. For example, during storage of the waterbath, the display 720 can be pressed into the housing 705 and the cover 730 can be lowered to provide a substantially fluid tight seal.

In other embodiments, the waterbath may include two or more separate covers that can be moved into place during storage or opened during use. One example is shown in FIG. 8. The waterbath 800 includes a housing 805, a first compartment 810, a second compartment 815, a display 820, a center handle 825, a first cover 812 coupled to the first compartment 810 and a second cover 817 coupled to the second compartment 815. In operation, the first cover 812 and the second cover 817 are folded to the position shown in FIG. 8. Suitable treatment parameters can be entered into the device and displayed on the display 820, and treatment can be provided by introducing a fluid into each of the first compartment 810 and the second compartment 815 and then initiating the treatment method. Once treatment is finished, the introduced fluid can be removed from the waterbath 800 using the center handle 825. For example, the waterbath 800 can be transported to a sink or bathtub and the fluid can be dumped into the sink or bathtub. Alternatively, the individual compartments 810, 815 can be removed from the housing 805 and each compartment can be dumped individually.

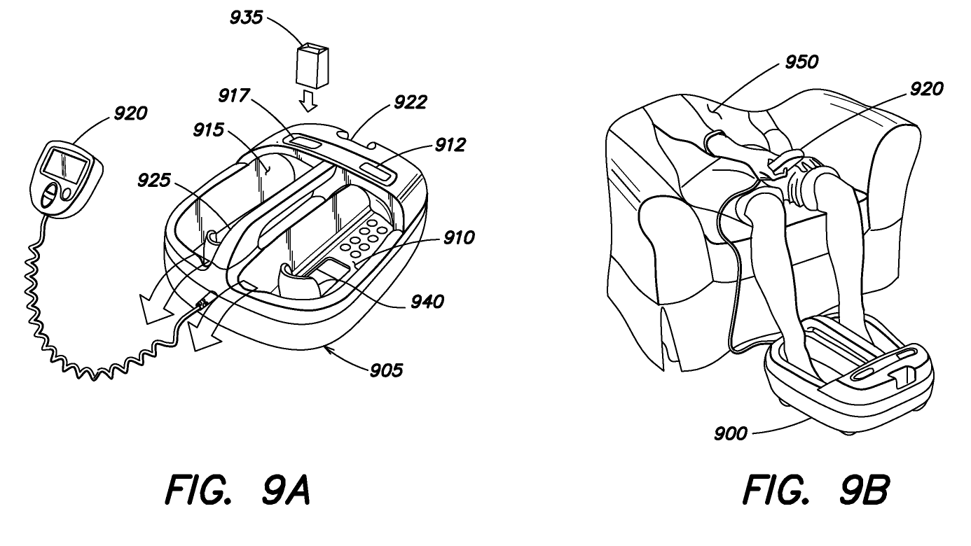

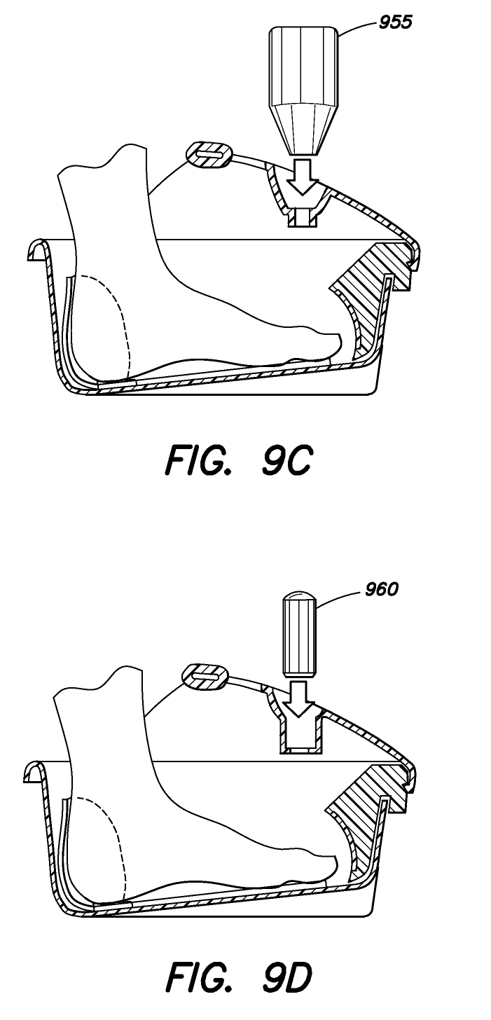

In certain embodiments, the waterbath can be configured with one or more ports or couplings to facilitate introduction of desired species into the waterbath. Referring to FIGS. 9A-9D, a waterbath 900 can include a housing 905, a first compartment 910, a second compartment 915, and a controller 920 coupled to the housing 905. Each of the compartments 910, 915 may include a respective port 912, 917 for introducing a species into fluid in the waterbath 900. For example, a prepackaged amount of a salt can be provided in the form of a small box or container 935. In an alternative configuration, the container 935 may include a pre-measured fluid volume that is introduced into the compartment to provide a desired amount of fluid and/or fluid containing a desired concentration of species such as salts or other chemicals. Notwithstanding the type of material included in the container 935, the ports 912, 917 can include a structural feature configured to cause the release of the contents of the container 935 into the compartment. For example, the port may include a point or protrusion that punctures the container. In other embodiments, the container and the port can each include a suitable fitting such that when the container is mated with the port, species from the container can flow into the compartment. Other possible configurations for releasing the species in the container 935 into the compartments of a waterbath will be selected by the person of ordinary skill in the art, given the benefit of this disclosure. In addition, the exact configuration of the container 935 is not critical. The container may take the form of a bottle 955 (FIG. 9C), a cylinder 960 (FIG. 9D), a fluid cartridge or other forms, such as solid tablets. In addition, the port can be located anywhere on the housing provided the contents of the container, when coupled to the port, can be released into one or more compartments of the waterbath 900. As described elsewhere, the remote 920 of the waterbath 900 can be used to program a treatment method. When not in use the remote 920 can be stored in a dock 922 on a front surface of the housing 905. For example, once the treatment parameters are initiated by a user 950 (see FIG. 9B), the remote 920 can be placed in the dock 922 so that a user does not have to hold the remote 920 during treatment. Where additives or fluids are used in the treatment methods described herein, indicators can be added to provide a visual indicator that the dispensed species has already been added to the water (to avoid risk of multiple doses). The exact volume or amount of material added to the compartments can vary depending on the compartment design, the amount and type of current to be delivered and the desired concentration of any species in the fluid.

In some embodiments, the waterbath shown in FIG. 9A can also include an adjustable heel stop 940. This heel stop 940 can be used to facilitate proper placement of a foot in the waterbath 900. The heel stop 940 is optional and may be omitted in the device shown in FIG. 9A or can be included in the other waterbath embodiments described herein. For example, a user can place a foot such that the toes of the foot contact an electrode toward the front surface of the waterbath 900. The heel stop 940 can then be slid forward to contact the heel. In some embodiments, the heel stop 940 can include one or more electrodes such that placement of the heel against the heel stop 940 brings the electrode into contact with the heel of the foot. In this manner, placement of the foot at desired position in the waterbath 900 can be performed. The integrated handle 925 permits placement of the footbath 900 at the desired area and/or also facilitates lifting of the footbath and dumping of any fluid from the footbath.

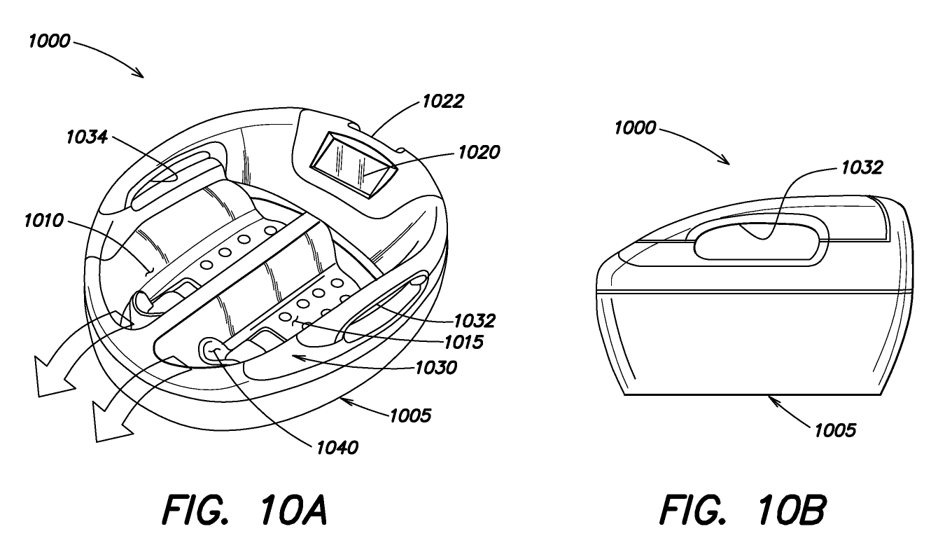

In certain embodiments, the waterbaths described herein can include two or more handles or other features designed to facilitate movement or portability of the waterbath. FIGS. 10A-13B show waterbaths having various features that increase the overall portability of the device. Referring to FIGS. 10A and 10B, the waterbath 1000 includes a housing 1005, a first compartment 1010, a second compartment 1015, a display 1020 and a remote dock 1022 and heel supports 1040. The housing 1005 has a tapered shape from front to back such that the height of the housing at the back is lower than that at the front. For example, the area 1030 of the housing 1005 typically has less height than the height of the area at the front of the housing 1005. The housing 1005 also has two integrated handles 1032 and 1034 The integrated handles 1032 and 1034 permit placement of the footbath 1000 at a desired area and/or also facilitate lifting of the footbath and dumping of any fluid from the footbath.

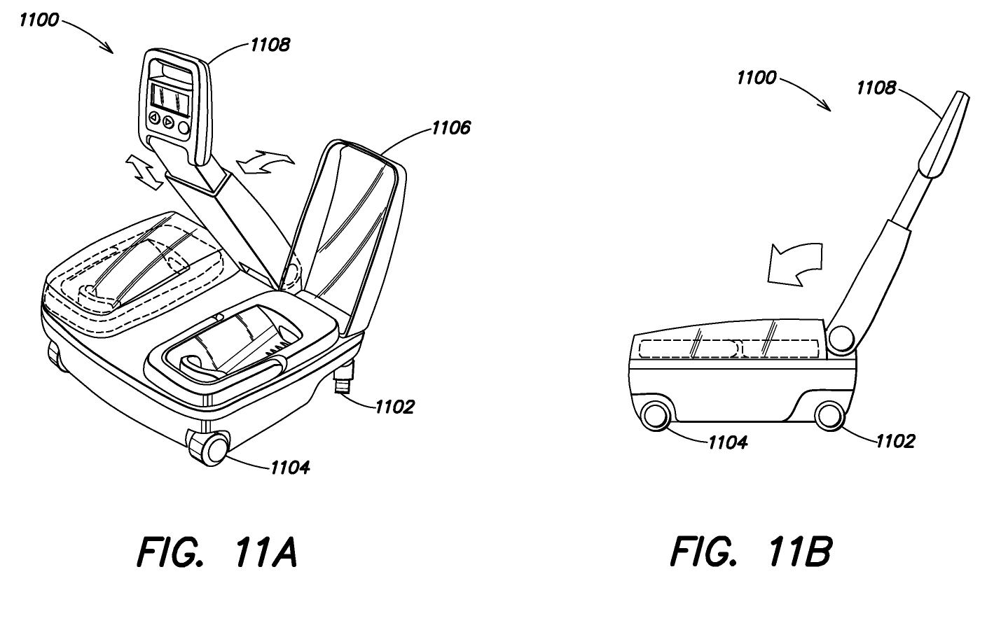

In other examples, the waterbath housing can include features to facilitate movement of the footbath when, for example, the footbath is empty or is full of fluid. Referring to FIGS. 11A and 11B, the footbath 1100 includes a set of wheels, which could be manual or powered, on each lower corner including wheels 1102 and 1104 to facilitate rolling of the footbath 1100. The footbath 1100 also includes a cover 1106 and a handle 1108. The cover 1106 can be lowered into place to retain the fluid within the waterbath 1100 during movement or storage of the waterbath 1100. For example, the cover 1106 can be lowered and the handle 1108 can be grasped to roll the waterbath 1100 to a desired position.

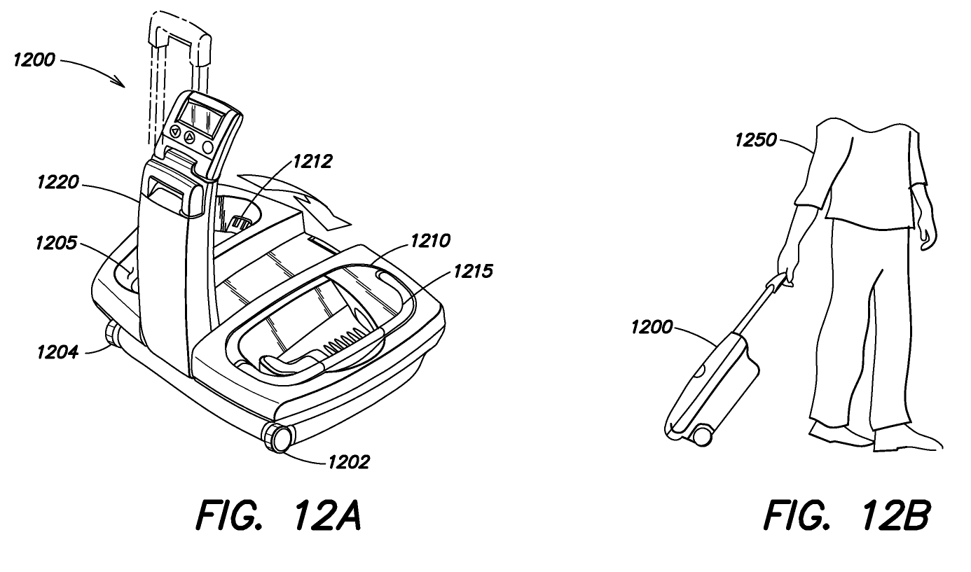

In additional examples, a waterbath can include a pair of wheels on adjacent corners of the waterbath. Referring to FIGS. 12A and 12B, the waterbath 1200 includes a first wheel 1202, a second wheel 1204 and a handle 1220. The waterbath 1200 also includes receptacles 1205 and 1210 each configured to receive a compartment such as the compartment 1215 shown inserted into the receptacle 1210. Shown in receptacle 1205 is a connector 1212 that is effective to couple one of the treatment compartments to the waterbath 1200 so that current can be provided to the compartment. The handle 1220 can be used to move the waterbath 1200 when a user 1250 tilts the waterbath 1200 and rolls it using the wheels 1202 and 1204. If compartments are inserted into the waterbath 1200 during movement, the compartments desirably include a cover that can be lowered into position to retain any fluid in the compartment within the waterbath 1200.

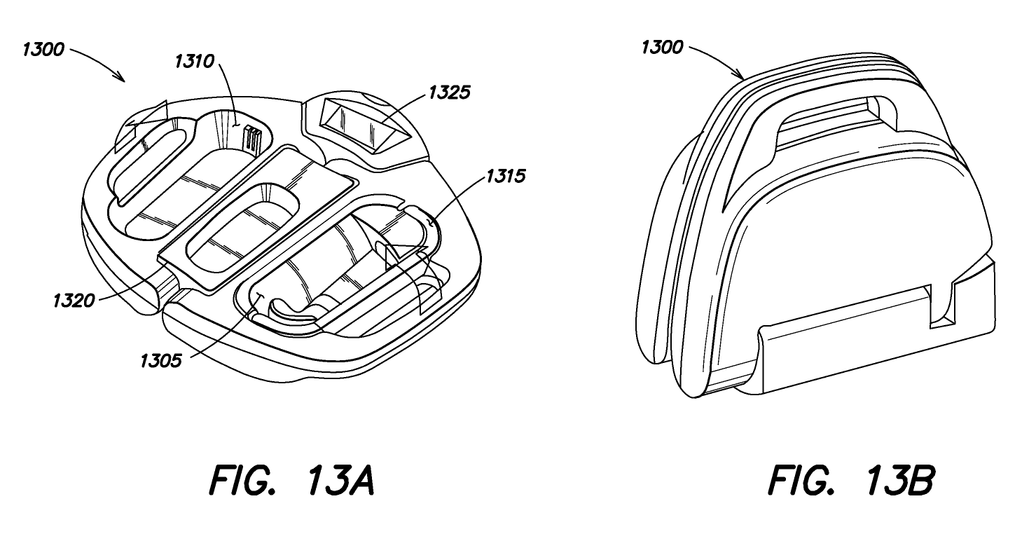

In certain examples, the waterbath housing can be configured to be portable. Referring to FIGS. 13A and 13B, a waterbath 1300 comprises two receptacles 1305, 1310 each configured to receive a treatment compartment such as a treatment compartment 1315. The waterbath 1300 also includes an insert 1320, a controller 1330, and a display 1325. The insert 1320 can be used to keep the waterbath 1300 in place and optionally to store a remote or other desired items. The waterbath 1300 can be folded by a medial hinge (or a hinge placed elsewhere) that connects the lateral sides of the footbath 1300 to each other (FIG. 13B).

In the waterbaths shown above, certain components can be interchanged with other components listed above without departing from the scope or operation of the waterbath.

Other Treatment Devices

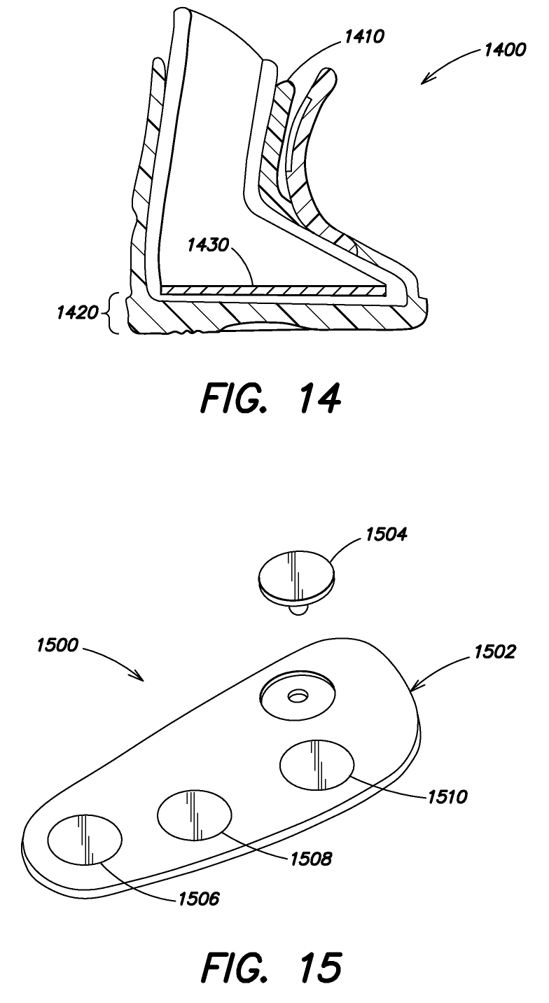

In certain embodiments, non-waterbath treatment devices can be used with the treatment methods described herein. In certain examples, a boot or shoe may be used to prevent and/or treat sensation loss. One illustration of such a device is shown in FIG. 14. The boot 1400 comprises a sole 1420 and an upper 1410. The sole 1420 may include two or more electrodes. For example, two or more electrodes may be embedded in an insole 1430 such that placement of a user's foot within the shoe results in contact of the foot with the electrodes. The upper 1410 may include a gel or fluid carrier that surrounds the foot or is otherwise in contact with some portion of the foot. The gel or fluid carrier may be contained within a membrane or other suitable material such that the foot itself does not get wet. The shoe typically includes two or more electrodes, which may be integral to the sole 1420. Such electrodes can be electrically coupled to an external power source, or the power source may be onboard, e.g., a battery, so that the user can be ambulatory during treatment.

In other examples, an insert may be placed in the shoe 1400. The insert may substantially conform to the inner surfaces of the upper 1410. The insert may take the form of a bootie or sock-like device that substantially envelops the foot and may include fluid trapped or saturated in a mesh or other material. In use, the insert can rupture such that the foot is in contact with fluid. In certain examples, once treatment is performed, the insert may be removed and discarded. In some examples, only certain areas or bladders of the insert may retain fluid while other areas remain dry or include air or other gas to provide increased comfort when the boot/shoe is being used.

In some examples, the insert may include electrodes or conductive areas where treatment is to be provided. Such conductive areas may be arranged to come into contact with an electrode in the sole of the shoe such that current can flow from the shoe through the insert and to the subject. For example, force from a user inserting their foot into the insert may force conductive areas of the insert into contact with conductive areas of the shoe such that current can flow from the shoe and to the insert.

In an alternative configuration, the insole 1430 may include a gel or fluid that is released when the foot is inserted into the shoe 1400. For example, a user may insert a foot and apply a force to the insole 1430 by standing. This force can rupture a bladder in the insole 1430 and release the gel or fluid such that the gel or fluid is in contact with an area of the foot to be treated. Similar to the insert, the insole may include one or more conductive areas that can come into contact with electrodes of the sole such that current may flow from the insole and to the area of the subject's foot to be treated.

In some examples, the shoe may include or be produced from a conformable or viscoelastic material having an open pore structure that can retain a fluid within the pores. Such material can provide for cushioning of the feet as well as acting as a carrier to retain the fluid. Conductive particles may be disposed in the material, e.g., either in the whole material or in selected areas of the material, such that current can flow to those areas of the feet. Such conductive particles include, but are not limited to, metals such as copper, silver, gold, etc., conductive plastics, semiconducting materials, organometallics, alloys, and other conductive species.

In some examples, the shoe may be a fixed size such that a user selects the size based on the overall length and width of each foot. In other examples, the shoe may be adjustable, e.g., include rear straps, such that one shoe may be used in two or more subjects with different size feet. For example, the shoe may have a sandal or “croc” configuration such that the overall dimensions between the front and back of the shoe (and/or the lateral dimensions) may be adjusted by the user. In some examples, the shoe may be sized and arranged similar to commercially available insoles, e.g., small, medium and large, such that a user can select the most appropriate size based on their foot size and small adjustments can be made to provide a tight fit. Alternatively, there may be no rear portion to the shoe such that the overall size of the sole is less critical. In such embodiments, the entire sole may be electrically conductive to provide current to substantially the entire bottom of the feet, whereas in other examples, areas of the sole may be electrically conductive.

In embodiments described herein that include one or more electrically conductive areas, other areas may include an insulator, such as rubber or other elastomers, to render them substantially non-conductive. In some embodiments, the sole or insole may include a plurality of removable insulated discs such that the user can remove selected discs to provide a conductive pathway to the foot. One illustration of such a device is shown in FIG. 15. The insole 1500 includes a plurality of removable discs 1504, 1506, 1508 and 1510. In one embodiment, the base 1502 of the insole may be nonconductive and the top of each of the discs may be nonconductive. When one of the discs is removed, the underlying area is conductive and can provide a current to the area of the body near that disc. In another embodiment, the base 1502 of the insole 1500 can be conductive and the top surfaces of each of the plurality of discs can be conductive. When a disc is removed, then that area becomes substantially non-conductive while other areas in contact with the insole can be provided a current. The insoles can be used with both waterbath and non-waterbath treatment devices to facilitate treatment of a desired area. In some examples, the circular or other shaped discs can include an adhesive that may be removed in certain areas that would lie against the foot to provide treatment to those areas. Such insulated discs may include protrusions or similar features to facilitate ease of removal from the insole or sole. The exact number of discs may vary and in certain instances at least three, four, five, ten, fifteen or more disks may be present. The exact dimensions of the discs may also vary with the cross-section being substantially the same as the area of the foot to be treated.

In certain examples, illustrative dimensions for an insole include but are not limited to those dimensions commonly encountered for human foot. For example, foot size for treatment area (height would be distance from floor to ankle bone aka malleoli) may be Men 95th percentile: width=4.2 inches, length=11.5 inches (this is a US size 12.5). Extra wide foot at this shoe size is 4.5 inches. Women 95th percentile: width=3.4 inches, length=8.4 inches, but with limb amputation, e.g., distal portion of foot could lower it to in the order of 6 inches. For example, the lower end of sizing is highly variable as there can be amputated limbs which would be exposed to the treatment. Other dimensions will be recognized by the person of ordinary skill in the art, given the benefit of this disclosure, depending on the exact shape of the discs in the sole or insole.

In other embodiments, an insole that is substantially conductive over its entire surface may be rendered non-conductive by application of an insulator. For example, a user may apply strips or shapes of a non-conductive overlay such that current is only provided to certain areas of the feet. Alternatively, a user may apply such overlay to the feet themselves to prevent or reduce current delivered to those areas of the feet.

In certain examples, the insole may include embedded electrodes, whereas in other examples, the insole may include apertures that permit the foot to contact one or more electrodes embedded in the sole of the shoe. For example, the electrodes may be embedded in the insole and coupled to a power source through the sole or other areas of the shoe. The insole may be custom fitted to the user to account for variations in foot dimensions, ulcerations of the foot, deformation of the foot or toes or the like. For example, a mold or impression of the user's foot may be produced and used to produce an insole designed for that particular person. Electrodes may be embedded in the insole during production such that treatment can be accomplished using the methods described herein to prevent and/or treat sensation loss. In an alternative configuration, conductive areas may be embedded in the insole that can mate with electrodes embedded in the sole to provide a conductive path to the feet.

In certain examples, the shoe shown in FIG. 14 is designed to provide for treatment by insertion of the foot into the shoe and application of a current using the shoe and either a power source on-board or external to the shoe. In some configurations, a shoe may be designed to function with a waterbath similar to the ones shown in FIGS. 1-13B. For example, the shoe may include a plurality of apertures or through-holes that are substantially continuous from the inner part of the shoe to the outer part of the shoe such that fluid from the waterbath can enter the shoe after insertion of the shoe into the waterbath. In such embodiments, there may be a conductive path from the waterbath to the shoe and to the foot to provide for treatment. The use of a shoe in combination with a waterbath may be particularly desirable where complete immersion of the foot in a fluid is useful for treatment, e.g., for treating multiple areas of the foot, ankle, etc. or for where a species in the fluid is desirably provided to many different areas of the foot, e.g., an emollient, moisturizing, debriding, antimicrobial, antibiotic or other agent is provided in the waterbath solution.

In certain embodiments, a device configured as a slipper may be used to provide treatment to prevent and/or restore sensation loss. In some examples, the slipper may be pre-soaked in a fluid such that the user need only insert their foot into the slipper. In other examples, the user may add fluid to the slipper prior to treatment. In either instance, such fluid can be tailored to meet the particular needs of the subject. For example, where the subject has one or more ulcerations on the foot, the fluid may include an antibiotic to prevent or treat a bacterial infection of or in the ulceration. Where a subject has a fungal infection of the foot, e.g., on the nails or skin such as Athlete's foot, the fluid may include an antifungal such as those commonly used to treat Athlete's foot, e.g., terbinafine, clotrimazole or miconazole. The fluid may also include surfactants, conductive species or other materials to provide desired properties.

In some examples, the slipper may be sealed in a foil or plastic pouch to prevent dehydration and/or contamination prior to use. During use, the slipper may be removed from the pouch and coupled to a power source, or the slipper may include an on-board power source such as, for example, a battery pack. The slipper can be configured to provide a single treatment or multiple treatments. To avoid contamination or user error, in certain embodiments, the slipper may be configured to provide treatment for a specified time at a specific current and using a specified waveform, any of which may be those illustrative treatment times, currents and waveforms described herein. For example, the user may activate the slipper by pressing a button or can activate the slipper by standing to force two or more conductors to come into contact. Once activated, a timing circuit or the like may be integrated such that treatment is not provided for too long of a duration. Where a power source is on-board, the power source may be configured to provide only enough power to provide a desired treatment time at a desired current. Such configurations facilitate ease of use and reduce the likelihood of user error.

In some embodiments, the slipper is configured for single use and may be disposed of subsequent to use. In other embodiments, the slipper may be used multiple times and can be stored, for example, in a suitable solution to prohibit bacterial or fungal growth. Where the slipper is configured for multiple uses, the material used to construct the slipper may be fabrics or other materials that can withstand repeated use. Where the slipper is configured for single use, the slipper may be constructed from paper, cardboard or other relatively non-durable materials that can withstand a single use but not necessarily multiple uses.

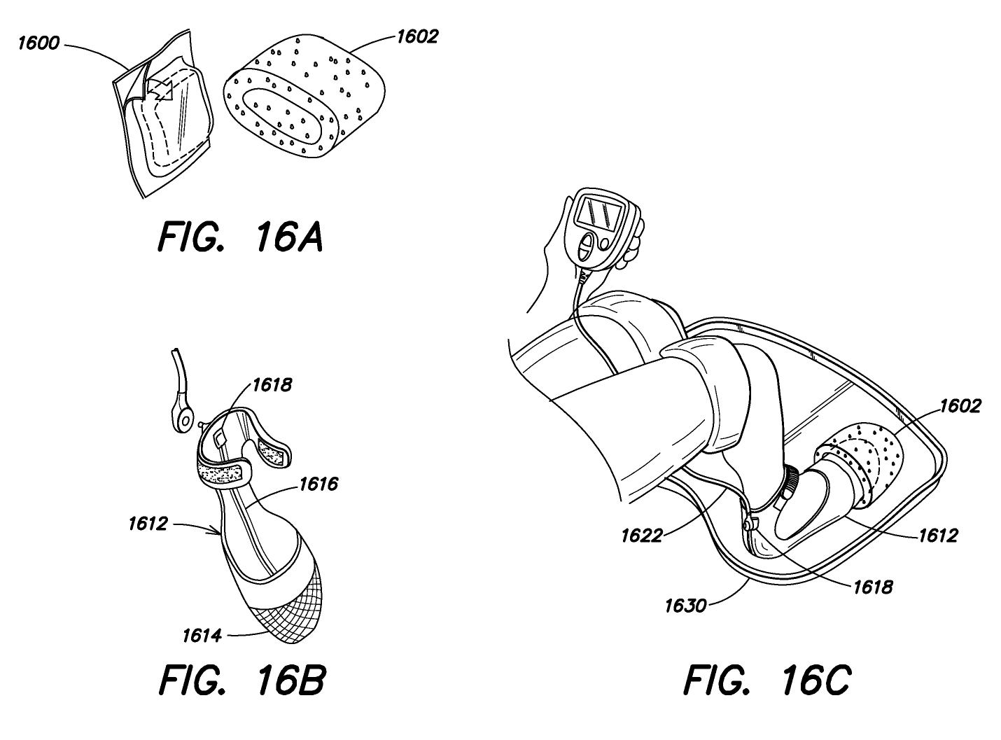

In certain embodiments, the treatment device may include or be a pad, spacer, or sponge soaked in a solution. The sponge can be used with the waterbaths described herein or can be used by themselves. For example and referring to FIGS. 16A-16C, the sponge 1602 can be sealed in a package 1600 that includes a fluid. By sealing the sponge in a package, such as a single use foil or other pouch with easy tear corner, or puncture disc, or screw cap, it can be easily opened by individuals that may suffer from conditions such as arthritis. The package can be shaped and weighted for easy holding in the hand. The package also compresses to a small volume when empty to take up less space in the trash. The sponge 1602 can be used with a shoe 1612 which includes electrodes embedded in it. For example, the shoe 1612 may include a conductive toe portion 1614, an internal wire 1616 and a connector 1618 on the heel that can be coupled to an interconnect to provide a current to the shoe 1612. In use, the sponge 1602 is placed around the toes and/or shoe 1612 (FIG. 16C), and an interconnect 1622 can be coupled to the shoe 1612. The sponge 1602 provides fluid to the toe portion inserted into the shoe 1612, and then a current can be provided to the shoe through the interconnect 1622. An optional mat 1630 can be used to avoid spilling any fluid on the surface which treatment is performed.

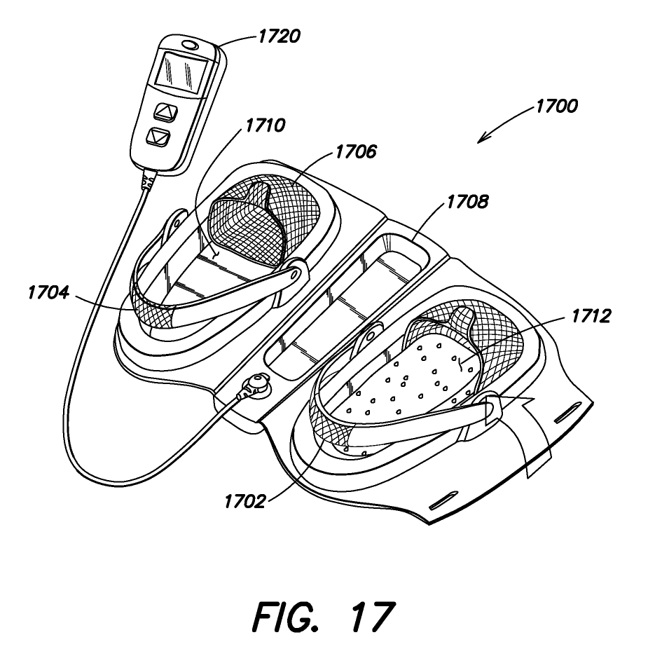

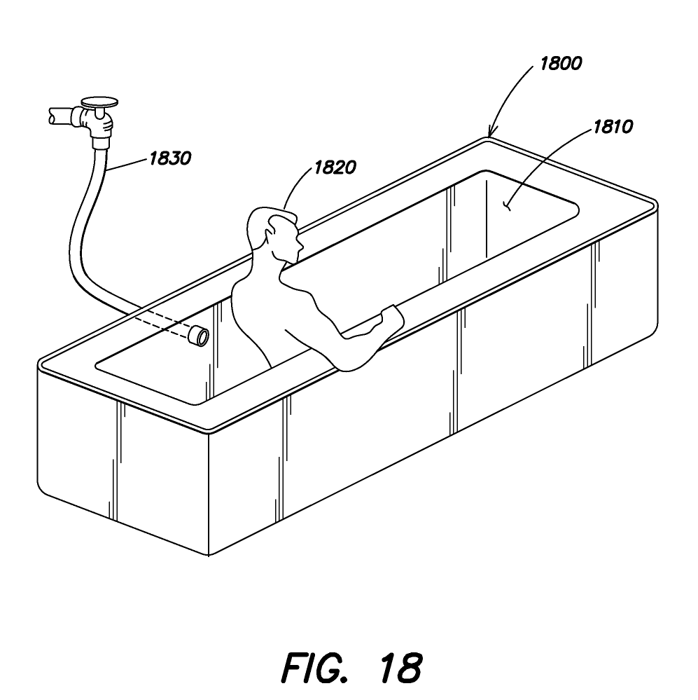

In other embodiments, the sponge may be sized and arranged to contact substantially all bottom surfaces of the foot. One example of a device including such a sponge is shown in FIG. 17. The device 1700 includes two adjustable heel straps 1702 and 1704 to permit placement of different size feet in the device 1700. The device 1700 also includes a conductive material 1706 attached to each of the foot wells, such as foot well 1710. A sponge insole 1712 can placed in the foot well and a foot can be placed on the sponge insole 1712. The adjustable heel straps 1702 and 1704 can include a conductive material which is operative as one of the electrodes, and the conductive material 1706 is operative as the other electrode. A user can place a foot onto the sponge insole 1712 and the heel strap 1702 can be tightened around the heel such that both electrodes are in contact with the foot. One or more treatment methods may then be applied to effectuate treatment of a desired area. A storage inset 1708 is provided to store the remote control 1720.

In certain embodiments, the devices disclosed herein may be configured to position the foot in such a way to provide a substantially similar amount of fluid between the foot and the electrode. In certain instances, the electric field provided to the foot may vary as a function of the thickness of fluid between the foot and the electrodes. To provide for a more uniform electric field distribution, spacers or positioners may be used in the devices such that the various areas of the foot are substantially the same distance from the electrodes. Such positioning can provide for more even treatment and reduce the likelihood that certain areas may experience local heating effects which could be harmful to the tissue.

Alternatively, where spacers or positioners are not suitable for use, one or more areas of the foot may be coated with a hydrophobic conductive material to increase the overall current provided to that particular area. Such hydrophobic conductive materials may include petroleum based jellies including a conductive metal or other species embedded or impregnated in the jelly. In other configurations, areas of the foot receiving too much current may be coated with non-conductive material to reduce the overall current delivered to such areas. Non-conductive petroleum based jellies and other non-conductive materials, or materials having a reduced conductivity, may be used for such purposes. Such materials may have the added benefit of inhibiting bacterial growth at areas or sites where the materials are applied.

In certain embodiments, rather than subjecting the user to placement of the foot in a waterbath, shoe or slipper, the subject may sit in a bathtub or other device. One illustration of such a device is shown in FIG. 18. The bathtub 1800 includes a fluid reservoir 1810 sized and arranged to receive an entire person 1820. Treatment may be accomplished by providing a current to the entire reservoir 1810 by, for example, integrating a plurality of electrodes into the fluid reservoir 1810, or may be accomplished by including electrodes in selected areas of the fluid reservoir 1810. For example, electrodes may be placed at one end of the tub such that one or more feet can be placed on the electrodes to provide treatment to the feet. As shown in FIG. 18, the bathtub 1800 may include a fill mechanism 1830 to facilitate addition of fluid to the bathtub 1800. A drain mechanism (not shown) may also be included, e.g., a drain connected to indoor plumbing, to facilitate easy removal of the fluid from the bathtub 1800.

In other embodiments, the treatment device may take the form of a shower having electrodes embedded in or added to the shower basin. The subject may stand in the shower and a continuous supply of fluid may be added by directing the shower water to the area to be treated. For example, the shower water can be directed to one or more feet that are placed on electrodes in the shower basin. In operation, the subject may enter the shower or just place the area to be treated in the shower. The use of a shower obviates the need to remove fluid from a basin once treatment is finished.

In some examples, the treatment may be targeted toward a specific area or portion of an appendage to be treated. For example, current may be preferentially provided, or provided in higher amounts, to a plantar surface of the foot. In some embodiment, one or more sensation loss tests, as described herein, may be performed to identify areas of the foot or other appendage where treatment is needed. Current can be delivered to such areas to effectuate treatment without delivering current substantially to areas that are not in need of such treatment. For ease of administration, areas not in need of treatment may be masked such that substantially no current is delivered to those areas, whereas areas to be treated may remain unmasked.

In some examples, the waterbath or treatment device may take the general shape of the appendage to be treated, e.g., foot-shaped to treat the feet, hand-shaped to treat the hand, etc. In such configurations, the waterbath may be produced in several sizes, to accommodate different sized feet, which minimizes or reduces the gap between the inner surface of the bath and the outer surface of the patient's foot so that the amount of bath solution required to cover the subject's foot, e.g., up to the ankle, is reduced or minimized. Since only a fraction of the bath solution is needed to fill the bath, carrying and emptying the device filled with solution is much easier for a subject, especially if they are elderly as are many diabetic subjects. Alternatively, filling a small container and carrying that container to the preferred treatment location is also much easier for the subject.

In certain examples, the opening of the shoe or foot sized bath is significantly smaller than the opening of a standard rectangular bath, and the device might also include a sealing cover which can be closed to prevent the filled foot bath from spilling during transit to or from the sink.

In certain embodiments, the devices used herein may be used in combination with one or more accessory devices designed to facilitate ease of treatment. For example, due to the limited mobility of many subjects with sensation loss, a hassock, ottoman or comparable device that includes a power supply and optionally space for storage may be included or used with the devices described herein. For example, the waterbath may be electrically coupled to the ottoman through a power cord or electrical leads to provide current from the ottoman to the waterbath. Similarly, where a shoe is used, the shoe may include inputs for receiving electrical leads coupled to the ottoman. The treatment protocol may be controlled using an interface of the ottoman to simplify the overall construction of the waterbath and/or shoe and to permit use of the ottoman with multiple different treatment devices. In some examples, the ottoman may also include storage space for retaining fluid, therapeutics or other materials or devices used in the treatment process.

In certain embodiments, the devices described herein may also be used with pre-filled pliable cartridges or pliable cartridges that can be filled by the subject. These pliable cartridges can be designed for connection into the foot bath to enable easy filling or emptying. The pliable cartridge filled with bath solution can be connected to the bath and squeezed to fill the bath or may enter the bath through gravity flow. When the bath requires emptying then the empty pliable cartridge can be connected to the bath and squeezed to draw the bath solution back into the cartridge which enables easy disposal of the cartridge and the used bath solution. In an alternative configuration, a small electric pump may be integrated into the inlet/outlet of the cartridge to pump fluid back to cartridge. In certain instances, the pliable cartridge design can include a sealing mechanism that ensures solution does not spill from the cartridge during filling, emptying or disposal. The pliable cartridge design may include a removable cap for easy filling by a subject.

In certain embodiments, one or more pre-determined quantities of materials may be added to the cartridge prior to or after filling as described herein. For example, pre-determined quantities of peroxide, therapeutics or the like may be placed in the cartridge prior to filling to facilitate addition of such materials by a subject.

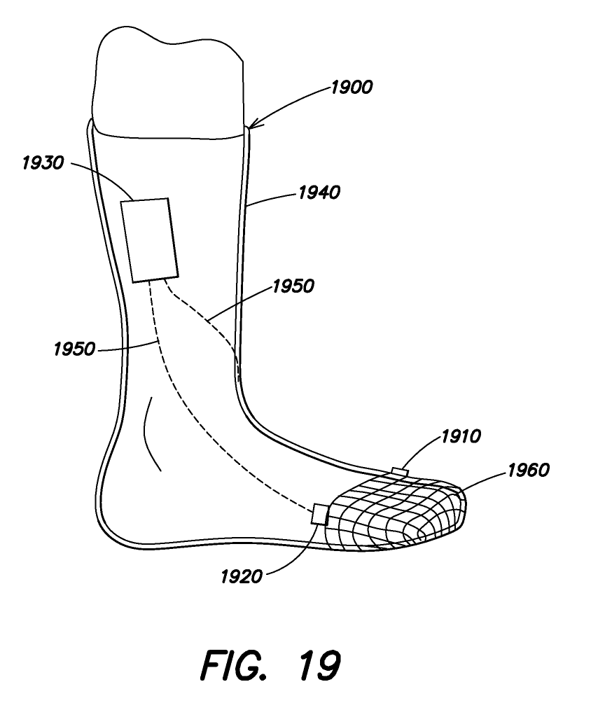

In certain embodiment, a wearable device such as a sock may be used to provide the treatment protocols described herein. The wearable device may be worn throughout the day or may be put on solely for treatment. One illustration of a wearable device configured as a sock is shown in FIG. 19. The sock 1900 may comprise electrodes 1910 and 1920 integrated into the sock 1900 and may include an on-board power source 1930 or may be designed to couple to a battery pack or other power source to provide the current for treatment. Where an on-board power source is present, it may be located on an upper portion of the shell 1940 of the sock so that the subject may still walk easily. Electrical leads 1950 may provide an electrical connection between the power source 1930 and the electrodes 1910 and 1920. In certain embodiments, a carrier 1960 that includes a fluid, gel or the like may be present and surround or be in contact with an area of the foot to be treated. In the embodiment shown in FIG. 19, the carrier 1960 is located proximal to the toes, but in other embodiments may be located elsewhere along the sock 1900. The fluid in the carrier may be retained in the carrier or may leak out of, or be forced out of the carrier, to contact the area to be treated. In embodiments where the sock includes an on-board power source, the power source may be a watch battery, 9V battery or other batteries. In some examples, the sock may include an accessory plug that can interface with a 12 V source, e.g., in a vehicle, or a 110 V source, e.g., in a domestic or commercial setting. Other power sources and voltages may also be used.

In certain embodiments, a treatment device may include a bandage that can be wrapped around the foot. In some examples, the bandage may include a conductive material and/or electrodes. In one embodiment, the bandage comprises a membrane or sleeve which may retain a fluid. In some examples, a scrim may be present on an outer surface of the bandage to reduce fluid loss. In certain examples, a power-source may be on-board, e.g., toward one end of the bandage, whereas in other examples, the electrodes may be coupled to an external power source. To treat and/or prevent loss of sensation, one or more of the treatment currents, time, protocols, etc. described herein may be used.

In certain examples, a treatment device configured as a glove may be used with the treatment methods described herein. The glove may take the form of a mitten or may include individual finger regions to separate the various fingers. In certain examples, the glove may include integrated electrodes that can couple to a power source that provides an effective amount of current to treat and/or prevent sensation loss.

In certain embodiments, the devices disclosed herein may include a means or device to provide fluid to the device. One such example is shown as fill-hose 1830 in the bathtub 1800 of FIG. 18. The use of a filling device may be particularly desirable to avoid the subject having to carry heavy amounts of water to the treatment device. In some examples, a hose or tube may be coupled to the treatment device For example, a first end of a hose may be coupled to a faucet and the second end of the hose may be coupled to a fluid reservoir to permit filling of the fluid reservoir. In other embodiments, tubing may be used to couple a fluid cartridge to a fluid reservoir such that fluid may flow into the fluid reservoir. For example, a fluid cartridge or bag comprising a fluid may be suspended from a device, e.g., a device similar to those used to retain intravenous fluid bags, and tubing may be used to connect the fluid bag to the treatment device so that fluid can be provided to the treatment device. In some examples, the fluid bag may be configured for placement on a belt or as a backpack so that the subject need not hold the fluid bag while fluid is being delivered.

Subsequent to treatment, it may be desirable to remove the fluid. This may be performed manually by dumping the fluid out or may be performed using other means. For example, one or more pumps in the fluid reservoir may be operative to pump the fluid to a sink, tube or drain for disposal. In other embodiments, a heating element may be used to evaporate the fluid. For example, a heating element may be integrated into the footbath and used to heat the fluid to facilitate evaporation. Such heating has the added benefit of humidifying the local area occupied by the subject. As discussed herein, aromatic species may be added to the water to provide an overall pleasant smell during treatment and/or during evaporation of the fluid. In other examples, the footbath may include a wick and a fan that provides air to the wick such that humidification is accomplished without heating. Combinations of pumping, heating and humidification may also be used. For example, a majority of the fluid may be pumped out of the fluid reservoir and any residual fluid may be heated to evaporation to dry the fluid reservoir.

Electrodes and Other Devices

In certain embodiments, the devices described herein can include two or more electrodes which can be placed in many different areas and have many different spacings. The exact electrode spacing of the device can vary depending on the treatment protocol and the particular device used. Where a waterbath is used, electrodes may be positioned on opposite sides of the waterbath. Where a shoe, boot or slipper is used, one electrode may be positioned at the top of the shoe, boot or slipper near the toes and the other electrode may be positioned at the rear of the shoe, boot or slipper near the heel.

In certain examples, the devices described herein can include a pair of electrodes or conductive areas that can electrically couple to a pair of electrodes. In some examples, a distal (toe) and proximal (heel) electrodes shaped for effective uniform distribution of energy can be used. In other examples, the spacing between an electrode and a tissue surface can be selected to provide for an effective uniform distribution of energy. For example, spacing in the order of 2-5 mm between the electrode and the tissue can be used. In certain embodiments, an adjustable electrode spacing can be implemented by the user by adjustment of the toe electrode, the heel electrode or both. In some examples, the electrodes can be shaped to follow the contour of foot to provide uniform spacing between the tissue and the electrode. The electrodes can typically be replaced by an end-user after a desired number of uses or at periodic intervals. In addition, the electrode materials are typically selected to be biocompatible materials to reduce toxicity and the potential of irritation. In some examples, one or more materials can be added between the tissue and the electrode to enhance treatment. For example, an open cell material, the voids filled with treatment solution such that electrical conductivity approximates the treatment solution. As discussed herein, the electrodes can be present in the fluid reservoir, a receptacle, a compartment, an insole or in combinations thereof, for example.

In some examples, the device may be designed such that the electrodes may have an infinite number of spacing positions between them by, for example making the electrode positions adjustable. Such adjustability may be accomplished by placing the electrodes in horizontal racks with a plurality of positions where the electrodes can lock into place. In some examples, the rack may be a smooth channel such that the electrodes can be positioned anywhere along the length of the rack. Where horizontal racks are used in a footbath, it may be desirable to include a removable plate that sits on top of the rack and where the subject's foot rests during treatment. For example, a removable plate, which may be conductive or include conductive areas, can be placed and rest on the electrodes to facilitate treatment. By way of example only, the heel spacer 1040 shown in FIG. 10A may have numerous positions, shown as depressions on the lower surface of the compartment 1015, such that a position of an electrode embedded in the heel spacer can be adjusted as the heel spacer is adjusted. Similarly, the position of the other electrode (or electrodes) in the device can be adjusted

In embodiments where a shoe, boot or slipper is used, the electrode spacing many be adjusted using insoles, by placing conductive strips in the sole or on the insole or using other positioning methods and devices. In other configurations, the electrodes may be positioned in the sole and attached using hook-and-loop fastener, an adhesive to permit removal and/or replacement by the user.

In certain embodiments, the composition of the electrodes is not critical and any conductive material including but not limited to graphite, stainless steel or other material can be used. In some examples, non-conductive materials can be coated with a conductive material to provide an electrode suitable for use. For example, lightweight polymeric materials can be coated with silver or other conductive metals to provide a lightweight electrode.

In certain embodiments, the devices described herein may include keyed or sized electrodes to prevent mismatch or misconnection of the electrodes to an external power source. For example, a power source having different shapes plugs for the cathode and anode may be used with electrodes including plugs shaped to fit into the cathode or the anode. Such a configuration permits ease of use in a home setting without worrying about misconnection of the various components.

In certain embodiments, during treatment it may be desirable to heat and/or cool the fluid to a desired temperature. For example, heating to a fixed safe temperature to stimulate blood vessel dilation and nerve regeneration may be performed to facilitate treatment and improve the overall outcome. Temperature control may be provided by regulating the temperature of fluid entering the device, by heating the fluid, by cooling the fluid or other means. The device may include integrated temperature control, temperature sensors and the like to prevent overheating or burning of the subject. In some examples, during administration of the treatment, heating and cooling cycles may be administered to assist in treatment. Such heating and cooling cycles may be performed using many different gradients, methods and cycle times. In addition, temperature sensors may be added to the area to be treated to monitor the temperature of the area and ensure that the temperature is not so high as to cause tissue damage.

In certain examples, the devices disclosed herein may include a conductivity sensor or device configured to measure the conductivity. During operation, the overall conductivity of a device may change or the conductivity adjacent to an area to be treated may be different than the area adjacent the electrode. As such, it is desirable to measure the conductivity near the area to be treated to make sure that the desired amount and level of current is actually being delivered to the area. Illustrative conductivity sensors are well known in the art and may be integrated into the devices described herein or may be added subsequent to use of the devices described herein.

In certain embodiments, the exact materials used to produce the treatment devices described herein may vary. In some examples, the materials desirably are not susceptible to bacterial or fungal growth or do not promote bacterial or fungal growth. The materials may be coated with an anti-bacterial agent or anti-fungal agent to reduce the likelihood of contamination. The electrodes used in the devices disclosed herein may be produced using many different materials including, but not limited to, stainless steel, conductive plastics, carbon, graphite and other materials. Where portability is desired, the electrodes are desirably produced from a lightweight material to facilitate movement by the subject.

In certain examples, during calibration or checking of the device prior to use, the conditions and/or state of the electrodes may be tested. For example, in a calibration check, the electrodes may be tested to make sure they have a characteristic impedance which can be measured in a particular configuration during a portion of the calibration process. Such calibration can be used to verify that the electrodes are still performing within specification and have not been damaged or otherwise reached the end of their useful life. In other examples, the number of uses of the electrodes may be written or recorded in a table or database to monitor the specific number of uses of the electrode or time used. When a limit is reached, the device may be programmed to not work and/or generate an error message prompting the user to replace the electrodes.

The treatment devices described herein may be used in combination with each other. For example, a sock may be used in combination with a footbath to accomplish treatment. The sock may include an integrated power source and the footbath may be used to provide fluid to the sock for treatment. Alternatively, a sock may be used in combination with a boot or shoe that provides fluid. Also, a boot or shoe may be used with the footbath. Other devices, e.g., a bandage, may be configured for use with two or more different devices. It will be within the ability of the person of ordinary skill in the art, given the benefit of this disclosure, to use two or more treatment devices to provide treatment for prevention and/or treatment of loss of sensation.

In certain embodiments, one or more creams, emollients or the like may be added to the area to be treated pre- or post-treatment. For example, due to the presence of a fluid during treatment, the area may wrinkle from continued exposure to the fluid. In some examples, creams suitable for use may be those that can that setup a membrane barrier to the natural water flow for the lower conductivity solution (the water bath) to the higher conductivity physiological saline solution in the tissue. A petroleum jelly based product, for example, could setup such a barrier. When such localized barriers are present, the alteration of current flow to selected areas may be promoted. For example, it is desirable that the current flow from the electrode(s) through the tissue versus traveling around the tissue to the other electrode. By using a barrier cream, in non-electrode areas current can be “forced” preferentially to targeted tissue areas. Suitable creams and materials will be readily selected by the person of ordinary skill in the art, given the benefit of this disclosure.

In certain embodiments, a substantially solid hydrated material pre-shaped with electrolytes may be used. For example, a gelatin-like high water content material which could serve as a “current carrier” may be used. The hydrated material can be cut, shaped or preformed to the shape of your foot so that it could be stored in a foil pouch to hold the moisture, then removed and placed into a reusable slipper, e.g., essentially a slipper in a slipper. There may be slits or apertures that are positioned in the outside slipper that guide positioning into the disposable slipper. The electrodes could be inserted into the internal gel slipper as well.

In certain examples, the treatment device may be a closed system in that the total fluid volume does not change before, during or after treatment. For example, fluid may be recycled or pumped to a storage basin for reuse. The fluid may be filtered or treated, e.g., with UV light, ozone, peroxides, hypochlorite, etc. to destroy any organisms in the fluid. After a selected number of treatments, the fluid may be discarded and replaced with fresh fluid.

In some embodiments, the treatment devices disclosed herein may include an in-line generator or in-line valving such that other species may be added to the fluid during treatment. For example, it may be desirable to add peroxides, ozone, oxygen or other gases, anti-microbials, anti-fungals and the like during treatment. Such species can be added directly to the fluid being provided to the treatment device to facilitate mixing. In addition, the level of such species may be monitored or periodically adjusted to ensure a substantially constant level during treatment. In some examples, a species, e.g., an aromatic species, may be added to increase the overall usability of the device. Such species include, but are not limited to, esters, fragrances, oils, perfumes, aromatherapy salts, tea tree oil, Vitamin E, herbals, lavender, etc. may be added.

In certain embodiments and as described elsewhere herein, the treatment devices may be designed to facilitate portability. For example, stackable components that can be stored easily may be produced or the treatment devices may include packaging or housing to facilitate transport and/or storage. In the case of footbaths and waterbaths, the housing may collapse or be foldable, deflatable or the like such that storage and transport is simplified. The overall footprint of the devices can be minimized to reduce the space occupied the by the device during treatment. In some examples, the fluid reservoir may be clear and/or colorless such that the area to be treated can be observed during treatment, e.g., the entire area of the foot can be seen during treatment. In other examples, a mirror coating may be included on the bottom surface of the footbath so that the bottom of the foot can be observed from above.

In certain examples, a visual indicator may be added to the treatment device to monitor the course of treatment. For example, electrochemically active species may be included that are oxidized or reduced to a noticeable color after a certain period. Once this color appears, the subject can stop treatment. Similarly, dye molecules or other species that diffuse at a certain rate may be used as a timer to indicate that sufficient treatment has been performed.

In certain examples, the treatment devices and methods disclosed herein may be used to treat two or more disorders. For example, the devices may be used to treat a fungal infection of the skin of the foot and to treat and/or prevent loss of sensation. In other configurations, the device may be used to treat neuropathy, as described, for example, in U.S. Provisional Application No. 61/057,162, the entire disclosure of which is hereby incorporated herein by reference, in combination with treatment of sensation loss. In other embodiments, the devices may be used to stimulate muscle tissue in combination with treatment of loss of sensation.

In certain embodiments, a foot scanner may be used in combination with the treatment devices disclosed herein to assess the effectiveness of treatment. The foot scanner may implement one or more of the assessment methods described herein in a substantially automated manner such that a subject can get feedback regarding the outcome of treatment. The foot scanner can be used on a periodic basis to determine if treatment is effective or to determine if treatment should be continued or discontinued.

Certain embodiments described herein use a fluid in combination with current to treat and/or prevent sensation loss. The particular nature of the fluid is not critical and may be water, gases or other fluids or materials. In place of or in addition to the fluid, other materials may also be used. For example, gels, creams, microspheres, sols, ball bearings, water saturated meshes and the like may also be used. Such materials may be placed in a carrier or mesh or may be applied directly to the affected area.

In certain examples, a carrier or mesh may be added to the device to provide for sustained release of a material. For example, a carrier or mesh may include hydrogen peroxide impregnated in the carrier. During use, the peroxide may diffuse out of the carrier and into the fluid. Alternatively, the carrier or the fluid or both may include a peroxide generator such that peroxide is generated continuously during use of the treatment device.

In certain embodiments, it may be desirable to include one or more species in the fluid that provide a visual indicator of current flow. For example, exciting phosphors, magnetic particles optionally coupled to a dye molecule or some other physical change may be used to monitor current flow during treatment.

In certain examples, the fluid may include an additive that fluoresces or is otherwise visible under ultraviolet or infrared light. Such additive may be used to monitor the fluid life with the disappearance of the additive indicative of a fluid change being needed. In other embodiments, the additive may be used as a proprietary indicator or to “color-code” the particular fluid for a particular use, e.g., treatment of multiple disorders or treatment of a single disorder.

As discussed in certain instances herein, the species may be added to the fluid inline, by manual addition, through the use of a gel capsule, through effervescent release, by rupturing a bladder or blister pack of through other methods. When added or ruptured, the amount of species added to the fluid is desirably within a selected concentration range such that little or no dilution need occur prior to treatment.

Illustrative additives that may be used include, but are not limited to, hydrogen peroxide, epsom salts, hydrosols, hydrogels, amino acids, colloidal silver, sodium bicarbonate, salicylic acid, Efferdent, oils such as tea trea oil and other suitable materials. The additives may serve to provide some therapeutic function, e.g., antimicrobial, antifungal, etc. or may be used for aesthetic reasons, e.g., to prevent wrinkling, soften the skin, etc.

In accordance with certain examples, the treatment devices described herein may be particularly suited for use with subject having one or more limbs amputated. For example, individuals with loss of protective sensation frequently sustain wounds that do not heal, ultimately requiring amputation. The devices described herein may be sized and arranged to accommodate limbs post-amputation. For example, the devices may be configured such that they are operable even if one or more toes, or some portion of the foot, have been amputated.

In certain examples, the treatment devices described herein may be used prophylactically for diabetic patients at risk of loss of protective sensation. For example, a subject may be tested using one or more of the sensation loss testing procedures described herein, and those subject having reduced sensation may be treated prior to actual loss of sensation. Such pro-active treatment may be particularly effective at reducing the likelihood of untreated ulceration of the limbs and potential amputation.

In certain examples, the treatment methods and devices disclosed herein may be used in combination with other sources and/or types of energy and in particular may be used in combination with electromagnetic energy. As used herein, the term “electromagnetic energy” is used broadly and is intended to include gamma rays (wavelength less than about 10⁻⁹ cm), X-rays (wavelength from about 10⁻⁷ cm to about 10⁻⁹ cm), ultraviolet light (wavelength of about 4×10⁻⁵ cm to about 10⁻⁷ cm), visible light (wavelength of about 7×10⁻⁵ cm to about 4×10⁻⁵ cm), infrared light (wavelength of about 0.01 cm to about 7×10⁻⁵ cm), microwave radiation (wavelength of about 10 cm to about 0.01 cm), radio waves (wavelength of greater than about 10 cm) and any wavelength or energy between these illustrative types of electromagnetic energy, e.g., sound waves in various forms or from devices such as ultrasound devices having a wavelength of about 1.5 mm. The exact form of the electromagnetic energy used may vary depending on numerous factors including the wavelength of the electromagnetic energy, the area to be treated, treatment times, dosage and the like. Illustrative forms and devices for providing electromagnetic energy to tissue for treatment are described, for example, in U.S. patent application Ser. No. 11/774,367.

In certain embodiments, combination treatment with different modalities, e.g., current and UV light may be particularly desirable for certain disorders. For example, a bacterial skin infection such as, for example, cellulitis, erythrasma, folliculitis, skin abscesses, carbuncles, Hidradenitis suppurativa, impetigo, necrotizing skin infections or Staphylococcal scalded skin syndrome may be treated. In other examples, a blistering disease such as, for example, bullous pemphigoid, dermatitis herpetiformis, or pemphigus may be treated. In yet other examples, the applicator may be configured to deliver electromagnetic energy to treat a fungal skin infection such as, for example, candidiasis, ringworm, tinea versicolor, tinea pedis or onychomycosis. In still additional examples, an itching and noninfectious rash such as, for example, contact dermatitis, atopic dermatitis, seborrheic dermatitis, nummular dermatitis, generalized exfoliative dermatitis, stasis dermatitis, perioral dermatitis, pompholyx, a drug rash, erythema multiforme, erythema nodosum, granuloma annulare, itching, keratosis pilaris, lichen planus, pityriasis rosea, psoriasis, rosacea, Stevens-Johnson Syndrome, toxic epidermal necrolysis or other dermatalogical disorders such as, for example, dry nail may be treated. In certain examples, parasitic skin infections such as, for example, creeping eruption, lice infestation, or scabies maybe treated. In yet other examples, a viral skin infection, such as molluscum contagiosum or warts may be treated. In other examples, psoriatic nail disease following nummular dermatitis can be treated.

In certain examples, the treatment methods and devices disclosed herein may be used with one or more therapeutics or other compositions designed to prevent or reduce the likelihood of reinfection. Illustrative materials include antibiotics, antifungals, tissue sealants, tissue barriers, benfotiamine, amica, eucalyptus, panthenol, tocotrienols and tocopherols (e.g., vitamin E) and the like. It will be within the ability of the person of ordinary skill in the art, given the benefit of this disclosure, to select suitable compositions and devices to discourage or prevent reinfection of a tissue. By applying current during treatment, the therapeutic, in certain instances, may be driven into the tissue to increase the overall uptake.

In some examples, once the subject has been treated, the foot may be dried using numerous methods. As many subjects who are targeted for treatment with the devices and methods disclosed herein may have limited mobility and flexibility, external drying apparatus may be used. In some examples, the waterbath itself may include an integral heat gun to provide for drying of the foot without manual labor by the subject. For example, a heat gun may be positioned on an external surface of the waterbath and the subject may the foot under the heat gun for a desirable period to remove any residual fluid or species.

In other examples, the user may place the treated foot in a composition designed to remove the water, e.g., one including drying agents and optionally one or more therapeutics, to facilitate removal of the fluid. Such composition may be placed in a footbath or other device.

In some examples, after treatment using the waterbath, the user may place their foot in a sock or other wearable device which can absorb the fluid from the user's foot. Such wearable device may also include antimicrobials, e.g., antibiotics, silver, etc., anti-fungals or other therapeutics or compositions that may be beneficial in treating disorders of the foot.

In certain embodiments, the foot may be dried manually by the user. For example, a towel or other device may be used to remove residual fluid from the foot.

In some examples, massage or manipulation of the area to be treated may also be performed simultaneously with, before or after delivery of current. For example, massaging of the feet could be provided to supply additional stimulation to the foot to assist with improved blood flow and muscle relaxation during treatment. Such massage may be performed manually by the user, e.g., using their hands, or may be performed by including vibratory devices or other suitable devices that can provide massage.

The devices described above may be used with any one or more of the illustrative treatment protocols, times and the like described below in the following section or other suitable treatment protocols and time.

Waveforms, Current and Controllers and Treatment Methods

The exact waveform, current types, and levels that are used to prevent or treat the sensation loss may vary and may be altered during the course of treatment of a particular subject if the subject is not responding favorably to treatment. Reference is made below to a controller, which is the device, including any associated circuitry or accessory devices, that provides the particular amount and type of current (and optionally electromagnetic energy) for treatment. In addition, it may be desirable to use different current intensities to treat and/or prevent loss of sensation at different areas. For example, the current parameters used to treat loss of sensation of the foot may not be the same as those used to treat loss of sensation of the hand.

In certain embodiments, the waveform used may be square, exponential, trapezoidal, sinusoidal, monophasic, biphasic, symmetric, asymmetric or may take other forms commonly used in electrical stimulation devices such as, for example transcutaneous electrical nerve stimulation or neuromuscular electrical stimulation devices.

Similar to the type of waveform, the type and intensity of the current can vary from body area to body area and from subject to subject. For example, the electrical current may be a DC current modulation which can have trapezoid, square and sinusoidal wave pulses from 0-150 volts with alternating and continuous pulses modulated at between 1-200 hertz and electrical current ranging from 0-50 mA, with a current range from 20-50 mA.

In certain embodiments, the following parameters may be used for treatment: an amplitude of greater than 10 volts, e.g., 10-150 or 10-300 Volts, a pulsed current up to about 50 milliamperes, a pulse width between 5-100 microseconds, and pulse spacing (from leading edge to leading edge) of about 100 to 250 microseconds. In certain examples, the pulsed current comprises pulse pairs with a frequency of between about 100 and about 200 Hertz. In some embodiments, the current can be provided as a square wave, or as close as possible thereto, having a substantially fast rise and fall time. Such square pulses may be monophasic, biphasic, used in pairs or may take other forms.

In certain embodiments, a signal generator can be used to provide a selected current to the devices and systems described herein. For example, a signal generator operative to generate a biphasic waveform comprising successive cycles each containing a positive and negative pulse can be used. In some examples, the biphasic waveform is continuous. By a “continuous” biphasic waveform, it is meant a series of cycles in which the leading pulses are equally spaced.

In certain instances, the mean pulse width for the forward and reverse pulses, and preferably the pulse width of each of the forward and reverse pulses, may be 6 microseconds or less, 4 microseconds or less, e.g., 3 microseconds, 2 microseconds, 1.5 microseconds, 1 microseconds, 0.75 microseconds or less, and optionally at least 0.01 microseconds, 0.05 microseconds, or 0.5 microseconds. In some embodiments, short pulse widths are preferred so as to increase the rate of change of the electrical field, e.g., for a given RMS current in the tissues.

Without wishing to be bound by any particular scientific theory, by increasing the rate of change of the electrical field, increased coupling to cellular structures involved in transmission of pain may occur. Such coupling can provide an analgesic effect in addition to the treatment and/or prevention of loss of sensation. The signal penetrates deep tissues, and it is believed that it may produce beneficial effects by producing changes that affect one or more processes that occur in the central and or peripheral nervous systems, for example the behavior of microtubules, the rate of release of certain ligands and or the responses to them by various ligand gated receptors. The signal may also have effects on the mobility of ions associated with the transmission of action potentials and act directly on other cell structures such as voltage gated channels in both the peripheral and central nervous system.

In some embodiments, it is desirable that the mean pulse voltage, and preferably the voltage of each positive and negative pulse, is at least 100V, preferably 150V and more preferably 200V. Optionally, the mean pulse voltage and/or voltage of each pulse has an upper limit of 500V, 400V, 300V or 250V, e.g., to meet safety requirements.

While all combinations of preferred voltages and pulse widths are specifically included and can be used with any of the treatment devices described herein, optionally, when the voltage (mean value or voltage of each pulse) is at least 100V, the pulse width (mean value or width of each pulse) is 6 microseconds or 4 microseconds or less; when the voltage is at least 150V the pulse width is 3 microseconds or less; and when the voltage is at least 200V the pulse width is 1.5 microseconds or less. Additional suitable voltages, pulse widths and the like are described in more detail below.

In certain embodiments, the pulse frequency, i.e., the number of forward and reverse pulses per second, may be at least 100 Hz, 200 Hz, 500 Hz, 1000 Hz, 1200 Hz or more. It is desirable that the duty cycle (the ratio of on time to “off time”) through one complete cycle should be less than 10% or 5%, preferably less than 2% or 1%, and greater than 0.1%, particularly where the biphasic wave is continuous.

In certain examples, each pulse in the biphasic wave preferably has a rapid rise and fall phase, e.g., is substantially rectangular, subject to capacitor droop. Preferably the edge rate exceeds 250V/microseconds, more preferably 500V/microsecond or 1000V/microseconds.

In some embodiments, a high pulse current during the pulse “on” time is used. For example the waveform may have a pulse current of at least 0.3 A throughout the pulse period. The current may vary over the pulse period due to capacitor droop, and may for example be 0.7 A-3 A at the start of the pulse, falling to 0.5 A to 2 A at the end of the pulse. The RMS current flowing through the patient is preferably at least 3 mA, preferably at least 6 mA and more preferably at least 10, 20, 30, 40 or 50 mA. In certain embodiments, the interpulse spacing may be varied during the treatment. For example, the spacing can be varied independently of the power level or current supplied by the device or the associated rate of change of the electrical field in the tissues. In some embodiments it may be desirable to reduce the level of sensation that is felt, so that there is low or no sensation, e.g., allowing the patient to sleep when the device is operating. Alternatively, it may be desirable to provide a mild sensation, as this can be comforting to the user, and can help to distract from aches and pains.

In certain examples, it may be desirable that the mean pulse phase width, and more preferably the pulse phase width of each forward and reverse pulse, be selected to improve the ability of the sensation level to be varied with the interpulse spacing. In embodiments where some sensation may be desirable, the interpulse spacing may be at least 5 microseconds, preferably at least 6, 7, 8, 9, 10 microseconds or 75-150 microseconds, e.g., 10 microseconds. In some examples, the interpulse spacing may also be varied to alter the wave harmonics. Without wishing to be bound by any particular scientific theory, the harmonic content of the wave may play a role in determining its treatment efficacy. For instance, the continuous wave form can produces a series of harmonics at frequencies spread widely over the spectrum, when compared to a burst wave form in which most of the wave energy is concentrated around a narrow peak.

In some examples, high mean pulse voltages may be used, e.g., at least 100V, 150V or 200V where sensation is more likely to be experienced.

In certain embodiments, the controller that provides the current may be operated by a subject such that the user selects a desired current level. Illustrative calibration methods to select such a level are described herein. In some example, the current level selected is high enough to provide some sensation but not so high that localized heating or tissue damage may occur. The user may also be able to select the pulse spacing, total treatment time and other parameters.

Alternatively, the control element may provide automated variation of interpulse spacing, for example rhythmical modulation, or automated random modulation, e.g., in a series of modulation cycles. This may be of particular benefit at modulation rates below 200 Hz, preferably modulation rates below 100 Hz or 50 Hz, and/or greater than 0.25 Hz, so as to modulate the sensory nerves within the physiological range. Moreover, since the carrier signal can be applied at well above the physiological range and with high peak voltages it may be of particular benefit when the power levels are fairly high so as to penetrate large volumes of tissues.

In certain embodiments, the controller comprises a suitably programmed processor, a manual control or circuit adapted to provide a continuous, rhythmic or automated random modulation. Alternatively, or in addition, the processor may implement one or more other treatment protocols.

In some embodiments, the controller can also be configured to vary the pulse voltage or pulse width or both, for example a control which would allow the base level of sensation to be set by the user.

In certain examples, the treatment protocol may use devices that comply with applicable safety standards. For examples, two international safety standards of particular relevance are IEC 60601-2-10, “Particular requirements for the safety of nerve and muscle stimulators” and the US standard AAMI NS4-1985 (Transcutaneous Electrical Nerve Stimulation). Key safety requirements of 60601-2-10 are maximum limits on output current (rms) of 80 mA at DC, 50 mA at 400 Hz, 80 mA at 1500 Hz and 100 mA above 1500 Hz (with a 500 ohm resistive load), the maximum pulse energy should not exceed 300 mJ and the peak output voltage should not exceed 500V. The key requirements of NS4 are resistive loads of 200 Ohms, 500 Ohms and 1 kOhms as the test loads. A resistive load of 500 Ohms is considered as the reference waveform for safety purposes. The minimum output for efficacy (with the controls at maximum) is either 7 microC per pulse or a complex waveform whose average stimulating component amplitude is at least 0.5 mA into a load of 500 Ohms. The maximum charge per pulse should under no circumstances exceed 75 microC into a 500 Ohms load. Maximum average current shall not exceed 10 mA, the limit for DC currents to reduce burns due to ionic transport.

In certain embodiments, the controller may be configured such that it does not generate a sustained harmful average current, particularly where the controller provides a desired voltage but is limited in current output, so that it cannot generate a dangerous current during the pulse time either by itself or in combination with the capacitors, nor provide a pulse of more than the safety value in the event of either software failure or single component failure.

In some embodiments, the device may include a safety device or circuit which operates to discharge the capacitors to 0 Volts in the event that the voltage in either pathway and/or the output current exceeds a predetermined limit, e.g., as detected by monitoring circuits implemented in hardware. In some embodiments, this device can be a Silicon Controlled Rectifier (SCR). In preferred embodiments, the device may also be operable by a microprocessor in the event of an error or shutdown identified by the microprocessor.

In some examples, the controller may include two or more independent circuits for monitoring the voltage and/or current delivered and also comprises means for comparing the measured values, thus enabling an error in either circuit to be detected, and optionally causing shutdown of the device.

In certain embodiments described herein, pulse trains may be administered to effectuate treatment. The pulse train may be either a continuous stream of pulses or bursts of multiple pulses followed by a delay during which there is no activity. The particular delay between pulse trains can be varied and may be different depending on the particular disorder being treated.

As previously discussed, limitation of charge delivered to the patient is a key consideration in the safety of the apparatus, and it desirably is less than the limit of 75 microC which is the value at which charge may be hazardous through the chest (AAMI NS4) and it should also not exceed 300 mJ per pulse (IEC 60601-2-10). Bus capacitors can be designed such that the total charge delivered to the patient can never reach dangerous levels even in the event of multiple component failure causing the entire stored charge to be delivered. The charge delivered to the subject can be calculated by adding the charge transferred in positive and negative cycles and the continuous output of the boost converter during the pulse time. For this reason, the boost converter cannot be sized to maintain the voltage on load during the pulse output. The arrangement of separate forward and reverse bus capacitors permits the forward and reverse pulses to deliver essentially identical charge to the patient despite the droop in the bus, thereby ensuring that there is no net DC current which prevents adverse reactions caused by ionic transport to one or more electrodes. In some examples, the output of the controller may be a square wave biphasic pulse. The SCR can also be operated externally by the microprocessor, thereby providing a means of discharging the DC buses in the event of shutdown or an error identified by the microprocessor. The output current sensor is shown in the output circuit in the figure.

The independent circuit allows the microprocessor to determine if there is a failure in the voltage control part of the boost converter, by comparing the voltage set point with the voltage reported by the independent circuit. In addition the system voltage reference is continually checked by the microprocessor against a further secondary reference. Further the microprocessor can perform another safety check by comparing the average output current of the boost converter with the average patient current.

In certain embodiments, the overall controller can be considered as three sub-systems: a) the power supply and output stage which is the means of generating and controlling the output waveform and also has limits for output parameters such as voltage and current implemented in hardware and a means of reporting the values of key parameters; b) any independent safety circuit which provides a secondary means of limiting the output parameters to safe values, and reporting measured values; c) a device configured to control the output level by reducing the output of the first circuit from its maximum safe value and a means of comparing the voltages and currents sensed by the two independent circuits, thereby identifying if there is an error in either circuit and causing shut-down of the device.

In other examples, an electrical stimulator such as a Rich-Mar device may be coupled to the electrodes and current pulses as described herein may be provided using this device.

In certain embodiments, one or more calibration steps may be performed by a user prior to treatment. In one illustration, the current level is gradually increased until the user has tingling or sensation in the area to be treated. The current is then set at this level, and the particular pulse sequence and waveform may be applied for a desired period. In some examples, the condition of the electrodes may be tested, for example, during calibration to ensure that the electrodes have a characteristic impedance. Such checking may be performed to verify that the electrodes are still performing within specification and have not been damaged or otherwise reached the end of their useful life.

In another configuration, calibration may be accomplished using specific current levels. For example, a user may select from two, three or more particular current levels and select the lower current level where tingling or sensation is noticed. Subsequent to selection, treatment may begin. In other example, a higher current level than the minimum level where sensation is observed may be used. For example, it may be beneficial to use higher current levels than the minimum perceptible current level. As such, the current can be increased, for example, by 5 mA, 10 mA, 15 mA or 20 mA, above the minimum perceptible level to effectuate treatment. Temperature sensors may be added to the tissue to ensure that the tissue is not heated above a threshold temperature, e.g., 50° C. or 52° C.

In certain examples, one or more algorithms may be present to calibration the device prior to use. This calibration may be independent of the calibration used to set the current level. For example, the calibration may be a start up calibration that sets the temperature, checks solution conductivity and other parameters and/or automatically ramps intensity until feedback or until a user-selected current level is reached.

In certain embodiments, the exact treatment time may vary. In particular, the selected waveform, current, etc. may be applied for 30 minutes during each treatment session. Treatment may be repeated twice daily, once each day, every other day, biweekly, weekly or at other selected intervals. After each treatment or periodically, sensation loss may be evaluated using one or more of the test described herein. If, there is no response to treatment, then the treatment intensity, frequency of the like may be altered. If still no response occurs, then the subject may be non-responsive to the treatment.

In certain examples, treatment may be discontinued after a total amount of energy or current has been delivered to the target area. For example, the treatment may be integrated to provide a total current per treatment (based, e.g., on 50 mA for 50 microseconds×2 phases×100 pulse pairs per second×30 minutes) times the number of treatments. Thus, it may be desirable that the subject be exposed to a total amount of current. Such total current exposure may occur for each treatment or may occur based on the sum of all treatments over a weekly or monthly treatment plan.

In some examples, the controller may include an interface comprising one or more buttons that can be pressed or selected by user, e.g., the remote control shown in certain figures accompanying this description. Such buttons may be sized and arranged for operation by finger touch, toe touch or touch using a wand or associated stylus. In particular, foot operation may be desirable due to the limited mobility and flexibility of many of the subjects in need of the treatment devices and methods disclosed herein. In some examples, the user interface may be programmed into a remote control, which may be wired or wireless, such that the subject can control the parameters by selecting buttons on the remote or keys on an LCD screen of the remote. Where such a remote is used, the power source and other components may be integrated into the device, and a signal can be sent from the remote to the device regarding the selected treatment or other parameters.

In certain embodiments, one or more compliance and/or verification features may be integrated into the controller. For example, treatment information may be stored onto a flash card and brought to a physician's office for review/revision or such information may be remotely transmitted to a physician's office. In some examples, the device may include a card slot such that a treatment regimen may be written to the card by a physician and the card can be inserted into the device by the user prior to treatment. In other embodiments, the treatment protocols may be onto a device which is then read and operated by device to allow for customizing treatment over a period of time.

Due to the elderly nature of many of the subjects, the user interface can be simplified and include, for example, large display characters, limited keys, tactile feedback, etc. In particular, by limiting the number of keys, number of buttons and/or number of different treatment protocols on the device, the likelihood of user error can be reduced.

In some examples, the treatment protocols described herein may be used to simultaneously treat neuropathy and sensation loss. Illustrative protocols for treating neuropathy are described below.

In certain embodiments, several methods may be employed to evaluate sensation loss to determine whether or not treatment is effective. One method is commonly referred to as the 10-g Semmes-Weinstein Monofilament (MF) test. In this test, monofilament exerts 10 grams of force when bowed into a C-shape against the skin for one second. Patients who cannot reliably detect application of the 10-g monofilament to designated sites on the plantar surface of their feet are considered to have lost protective sensation. This loss of protective sensation is not equivalent to the total absence of sensation.

Patients with diabetes who have lost protective sensation as measured by standardized testing with the 10-g monofilament are at significantly increased risk to develop a foot ulcer that can lead to subsequent lower extremity amputation. Patients who have lost protective sensation are candidates for regular podiatric care, intensive foot care education, visual inspection of the feet at every office visit, and in some cases, therapeutic footwear. In addition, patients who have lost protective sensation may benefit from using the methods and devices described herein.

Testing for quantitative vibration perception threshold with an instrument called the biothesiometer is another excellent test for protective sensation, but the equipment is seldom available in primary care settings. Some clinicians believe that testing vibration sensation with the 128-Hz tuning fork over the hallux of each foot may detect loss of protective sensation equally well as compared to 10-g monofilament testing at four plantar sites on each foot. Although this has not been proven by an adequately powered prospective study, the 2006 “Clinical Practice Recommendations” of the American Diabetes Association propose that the use of both 10-g monofilament testing and vibration sense testing at the hallux may increase diagnostic ability to detect the loss of protective sensation. Suggested techniques for 10-g monofilament testing and vibration sense testing with the 128-Hz tuning fork are detailed below.

One protocol for the 10-g monofilament test is as follows: (1.) Obtain two or more reusable monofilaments or a packet of disposable monofilaments (MFs). (a) Use the 10-g MF<100 applications/day, then “rest” it for 24 h—thus the need for at least 2 MFs; (b) The accuracy of 10-g MFs obtained as samples from pharmaceutical companies is unknown. (2.) Check the 10-g ME for defects. Replace if bowed, kinked, or twisted; (3.) Compress the 10-g MF twice before use each day; (4.) Place the patient in the supine position for ease of testing; (5) Tell the patient that you are testing for loss of protective sensation that increases the risk for foot ulcer and amputation; (6). Demonstrate buckling of the 10-g MF on the patient's forearm or hand; (7.) Have the patient close their eyes; (8) Test four sites on each foot in random sequence. Avoid scars, calluses, and ulcers; (a.) Test the plantar surface of each great toe; (b.) Test the plantar surfaces of the 1st, 3rd, and 5th metatarsal heads of each foot; If callus, scar, or ulcer is present, test at adjacent sites on the plantar surface of the foot; (9.) Hold the 10-g MF perpendicular to the test site, and then bow it to a C-shape for one second; (a.) Do not allow the 10-g MF to slide along the skin; (b.) The patient should sense the 10-g MF by the time that it bows; (10.) Grade the patient's response by using the approach suggested by the International Consensus Group on the Diabetic Foot: (a) Bow the 10 g MF at a designated site, and ask the patient, “Do you feel it touch you yes or no?”; (b.) Repeat testing twice at each site and randomly include a “sham” application in which the 10-g MF is not applied. There will be a total of three applications at each site, one of which does not touch the skin; (c.) Protective sensation is considered to be present if the patient correctly answers two or more of the three applications, one of which was a sham; (d.) If the patient correctly answers only one or none of the three applications, return and retest that site; (e.) the patient is considered to have insensate feet if they fail on retesting at just one or more sites on either foot; (11.) Caveats: (a.) The feet may be falsely insensate when cold or edematous; (b.) Heel testing does not discriminate ulcer formers; (c.) Patients who have normal protective sensation should be retested annually; (d.) Technically, patients who have insensate feet need not be retested. Some clinicians believe that repeated testing of the individual with insensate feet may be a useful educational and motivational tool.

In certain embodiments, a tuning fork may be used as follows: 1. Use only the 128-Hz tuning fork (TF); 2. Demonstrate the sensation of vibration and its differentiation from pressure by applying the TF either to the wrist or elbow during and after stopping vibration; 3. Ask the patient to close their eyes; 4. Test the dorsum of each hallux (first or great toe) just proximal to the nail bed. Place the index finger of the other hand beneath the patient's toe to feel the vibration and determine the accuracy of the patient's response. Apply the TF perpendicularly with a constant pressure; 5. Use an initial sham test on each foot by applying a non-vibrating TF to be sure the patient does not mistake the sensation of pressure for vibration: “Is the tuning fork vibrating?” The patient should answer, “No.” 6. Use the “on-off” method to score the patient's response: (a.) Conduct testing twice on each great toe. (b.) On each test: Ask the patient to identify the beginning of the vibration sensation: “Is the tuning fork vibrating?” Ask the patient to identify the cessation of vibration on dampening the TF: “Tell me when the vibration stops.” Dampen the TF at random times without the patient's knowledge. (c.) The number of correct responses may vary from 0 to 8: vibration and cessation of vibration, each performed twice on each hallux. (d.) At least five incorrect responses rules in a diagnosis of peripheral neuropathy.

In some examples, a biothesiometer may be used to evaluate perception. A biothesiometer is an apparatus designed to measure the threshold of appreciation of vibration in human subjects. It is essentially an “electrical tuning fork” whose amplitude may be set to any predetermined level or whose amplitude may be gradually increased until the threshold of vibratory sensation is reached. Conversely, the amplitude may be lowered until the vibration is no longer discernible. In all cases the amplitude may be determined at any given level with a high degree of accuracy. A biothesiometer is available from Bio-Medical Instrument Company (Newbury, Ohio) or Diabetica Solutions (San Antonio, Tex.).

In some examples, nerve conduction velocity testing may be performed to evaluate treatment. This testing is typically used as a diagnostic for nerve damage, but is not necessarily correlated to loss of protective sensation (i.e., improvement in NCV does not necessarily infer improvement in sensory perception).

In other examples, a biphasic faradic pulse sequence can be used. Such a pulse sequence may treat and/or prevent loss of sensation, may induce muscle contractions, both or may provide additional treatment benefits. For example, a high phase charged system can be electronically pulsed and adjusted to induce deep-layered muscle contractions, causing greatly increased flow rates of both blood and lymphatics, patency of vessels permitting, and forcing blood into the microcirculation of the treated tissue. At the same time, the pulsing can treat and/or prevent loss of sensation. Alternatively, a different pulse, e.g., monophasic or biphasic waveform, may also be applied to treat and/or prevent loss of sensation while the biphasic faradic pulse sequence is used to induce muscle contraction and/or increase circulation. Without wishing to be bound by any particular scientific theory, biphasic faradic pulse sequence may stimulates angiogenesis, that is, budding of new capillaries and generation of denser capillary networks in the tissues. This lays the groundwork for new tissue growth and repair in the healing process. The pulsing may also increase the metabolic rate in the treated tissues, which can assist the intimal lining of the arteries to metabolize the excess unused nutrients clogging them. Whatever the actual cause, one resulting effect is improved blood flow.

In certain examples where a biphasic faradic pulse sequence is used, it is believed that the benefits of electro-stimulation are related to the stimulation frequency components (see, e.g. Savage, Brenda, “Inferential Therapy,” Faber and Faber, London, 1984). In some examples, the biphasic pulse period can be reduced to a lower limit with the pulse width at an upper limit There is one dominant frequency component with minimal high frequency content, as might be expected, considering that the corresponding biphasic pulse would begin to approximate a pure sinusoid.

In certain embodiments, there may be an optimum frequency for each type of tissue or for each type of disorder. Such frequencies may be, for example, programmed into the devices described herein such that a user can select the particular tissue or area to be treated and the device can provide that frequency during treatment. In some examples, the frequency may be between 0 and about 130 Hz. Frequencies between 5 Hz and 25 Hz are particularly desirable where biphasic faradic pulse sequences are used, whereas desirable frequencies for other waveforms may range, for example from about 75 to about 125 Hz.

In one example using a biphasic faradic pulse sequence, the following parameters may be used: load of 50 Ohms, a sequence of biphasic pulses, with a sequence duty cycle of 1.5 seconds on and 1.5 seconds off, a biphasic period of 17.5 ms and a repetition frequency of 57 Hz. In about 1.5 seconds, about 86 biphasic pulses to the patient will be delivered. The positive half of the biphasic pulse has a zero-to-zero pulse width of 110 microseconds. The leading edge has a 10 to 90 percent rise time of just under 8 microseconds. The trailing edge has a fall time of 9 microseconds. The negative going half has essentially the same characteristic as the positive going half.

In certain examples, the components of a machine that can implement a biphasic faradic pulse sequences are described, for example, in U.S. Patent Application No. 2007/0299482 published on Dec. 27, 2007, the entire disclosure of which is hereby incorporated herein by reference. In one example where treatment may be implemented using a biphasic faradic pulse sequence, in a first step it may be determined whether or not the treatment is suitable for the patient, e.g., patient selection may be performed. This protocol is for the treatment of any condition that can benefit from enhanced healing and repair through the mechanisms of increased blood flow, nutrient supply, waste removal and cellular activity.

The patient can be placed in a comfortable position, lying or sitting so the area of treatment can remain relaxed. Allow the area of treatment to be exposed, without pressure from the weight of the limb or body, to allow the stimulation of the circulation by the treatment. Typically, the choice of electrodes may be round, on the order of one to four inches in diameter, or rectangular on the order of one by two inches to eight by twelve inches, though different configurations and sizes may also be appropriate for specific body contours, as described elsewhere herein. In some examples, one to four pairs of electrodes surrounding the area can be used such that each pair causes the current to flow through the area of treatment. The electrodes may be secured with just enough pressure to cause full contact with the skin but not too much such that blood flow might be compromised to the area. Adhesives, gel carriers and other materials, as described herein, may be used to facilitate coupling of the electrodes to a particular area.

While treatment time may vary from person to person when biphasic faradic pulses are used, the duration of treatment may be, for example, 30-45 minutes twice a day, but a single 30-45 minute treatment 5 days a week can also effective. The typical treatment condition would be a severe diabetic ischemic foot ulcer that is in jeopardy of amputation. This affliction can require several weeks of treatment at 30-45 minutes twice a day. Conditions like carpal tunnel syndrome can require about 10 treatments over a two week period while conditions like acute sprain can respond in fewer treatments.

In some examples, all of the electronic parameters may be programmed into the device and only the intensity is varied. When placing the electrodes on the patient at the beginning of the treatment, the device can be turned off. With the power switch on, treatment begins by setting a timer. The initial intensity of current can be set with incremental adjustment upward the current on each set of electrodes as the patient tolerates over the first 5 minutes. The patient can develop a rapid tolerance to the current and there can be a decrease in the impedance of the tissues to the current as the body adjusts to it. 4-5 initial adjustments may be desirable. Readjust upward to tolerance after the first 10 minutes of the treatment.

In some examples, the intensity may be adjusted until visible muscle contractions are achieved. If there is a significant degree of disuse atrophy, active observable muscle contraction may not occur during the initial treatment session. In addition, considerable edema may make it difficult to observe muscle contraction. If there is no perception of contraction either by observation or by palpation of the muscle compartment, after the treatment is underway, then the electrode contact points may need to be checked for inadequate conduction of current. Repositioning may be necessary or a conductive material may be applied to the electrodes to achieve the desired results. When a patient has extreme neuropathy and claims to feel no electrical current, then the operator may check the integrity of the electrodes by turning down the intensity and applying it to the back of his or her own hand. The intensity setting of each channel being utilized can desirably be increased to the highest setting that the patient comfortably tolerates to enhance the effectiveness of treatment. At the highest tolerable setting, if there are very robust active muscle contractions, the operator may opt for decreasing the intensity slightly to avoid fatigue and soreness, particularly in the initial few treatments.

When the 30-45 minute treatment period is over, a buzzer will sound or the device can be configured to automatically shut off. Then all the intensity switches should be turned off or in the alternative, the timer may be coupled to a circuit that automatically turns off all the current intensity and includes a delay such that no additional treatment can be performed for a desired period.

In addition to the conditions that may be treated as described above other conditions suitable for treatment with the devices and methods disclosed herein include, but are not limited to, neuropathy such as, for example, diabetic neuropathy, peroneal palsey “drop foot,” Bells palsey of the face, trigeminal neuralgia, sciatica, HIV neuropathy, tarsal tunnel syndrome, alcoholic polyneuropathy, hereditary progressive muscle disease, hereditary progressive muscle dystrophy, paresthesia feet, paresthesia hands, ulnar nerve lesions, foot neuroma metatarsals, chemotherapy induced neuropathy, neuropathy of pernicious anemia, chronic pain syndromes, low back pain, upper back pain due to fibromyalgia, chronic tendonitis, painful shoulders or neck, diabetic ulcers of the toes, heel, calf, tibial surface or plantar surface, venous insufficiency, stasis ulcers pressure ulcers in immobile patients, ischial tuberosity bone fractures, “marching fractures,” “diabetic fractures,” avulsion fracture distal fibula, femur mid shaft fractures, femur impacted head fractures, radial head fractures, humeral head fractures, humeral mid shaft fractures, navicular fractures in the wrist, traumatic compression fracture in the lumbar spine, traumatic compression fracture in the thoracic spine, osteoporosis, osteoarthritis, or degenerative joint diseases, spontaneous compression fractures in the lumbar spine, spontaneous compression fractures in the thoracic spine, chronic hip pain from osteoporosis, degenerative arthritis knee, degenerative arthritis hip, degenerative arthritis ankles, osteoarthritis hand, generalized bone healing, heel pain, ischemic rest pain due to arterial insufficiency, feet, calf or thigh disuse atrophy, bedfast conditions such as lower and upper extremity wasting, muscle wasting conditions such as multiple sclerosis. muscle atrophy, Parkinsonism dementia paraplegia and quadriplegia, ischial tuberosity decubitus from a wheelchair, repetitive stress syndromes such as, for example, carpal tunnel syndrome, lateral epicondylitis (Tennis elbow), medial epicondylitis (golfers elbow), plantar fasciitis, costochondritis traumatic peripheral nerve injuries, sports injuries and acute sprains/strains such as, for example, ankle lateral sprain first or second degree, knee strain medial or lateral collateral ligament, wrist or shoulder strain, elbow, neck acute cervical strain, pulled hamstring, localized second and third degree burns, post radiation burns ulcerated or poorly healing, stasis ulcers due to venous insufficiency, post polio syndrome, lymphadema, post radiation treatment trauma, malignant tumors in conjunction with chemotherapy, bacterial infections including those causes by methicillin resistant Staphylococcus (MRS), Reynaud's Syndrome, chemotherapy-induced peripheral neuropathy, shingles (Herpes Zoster), relaxation of muscle spasms, prevention or retardation of disuse atrophy, increasing local blood circulation, muscle re-education, immediate post-surgical stimulation of calf muscles to prevent venous thrombosis, and maintaining or increasing range of motion, and the like.

Methods of treating and/or preventing loss of sensation may include, for example, identifying and/or selecting a subject having loss of sensation, and treating the loss of sensation using one or more of the treatment devices described herein. In other examples, methods or treating a diabetic subject having reduced sensation can include identifying and/or selecting a diabetic subject having reduced sensation, and preventing further sensation reduction by treating an area of the diabetic subject using one or more of the treatment devices described herein. In some embodiments, methods for treating loss of sensation can include identifying and/or selecting a subject having loss of sensation in a foot, and a administering an effective amount of current to the foot to treat the sensation loss using at least one of the treatment devices described herein and at least one of an amplitude of greater than 10 Volts, 10-150 or 10-300 Volts, a pulsed current up to about 50 milliamperes, a pulse width between about 5 and about 100 microseconds, a pulse spacing (from leading edge to leading edge) of about 100 to 250 microseconds, a pulse pair frequency between about 100 and about 200 Hertz, a square pulse, or a monophasic or a biphasic waveform. In further examples, methods for treating a diabetic subject having reduced sensation in a foot can include identifying and/or selecting a diabetic subject having reduced sensation in a foot, administering an effective amount of current to the foot to prevent further sensation loss using one or more of the treatment devices described herein and at least one of an amplitude of greater than 10 Volts, 10-150 or 10-300 Volts, a pulsed current up to about 50 milliamperes, a pulse width between about 5 and about 100 microseconds, a pulse spacing (from leading edge to leading edge) of about 100 to 250 microseconds, a pulse pair frequency between about 100 and about 200 Hertz, a square pulse, or a monophasic or a biphasic waveform. Methods of facilitating treatment by providing one or more of the treatment devices described herein can also be performed.

Certain specific examples are described below to illustrate further the novel embodiments and features described herein.

### Example 1

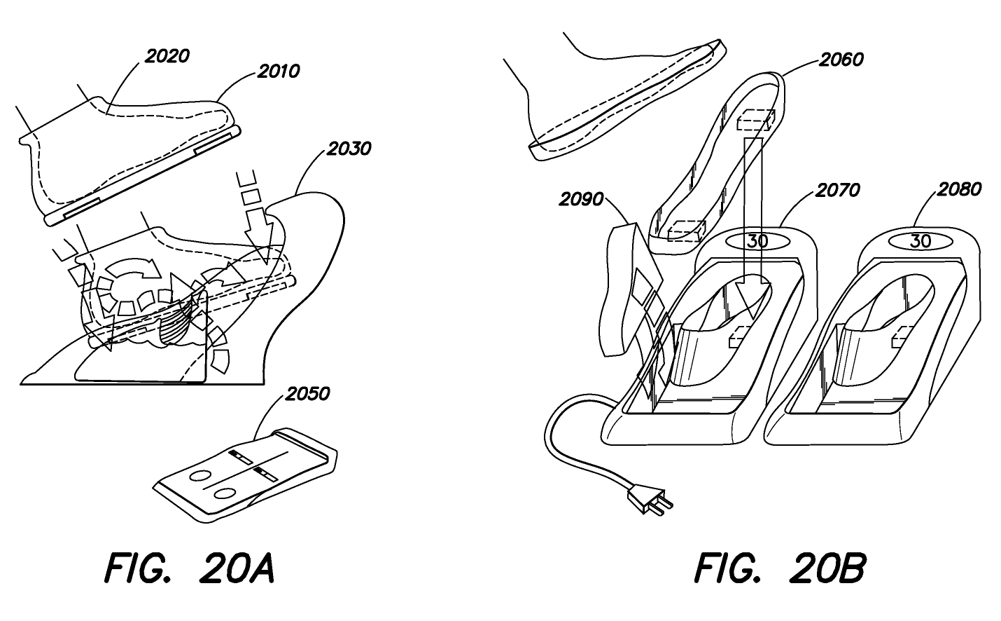

Referring to FIG. 20A, a slipper 2010 is shown that is constructed and arranged to receive a user's foot 2020 for treatment. The slipper 2010 may be designed for placement into a treatment device 2030, e.g., one or more of the footbaths described herein, such that treatment may be provided using one or more waveforms such as, for example, those described herein. The treatment device 2030 may be controlled remotely using the remote 2050.

### Example 2

Referring to FIG. 20B, an insole 2060 may be used to receive a subject's foot. The insole 2060 may be sized and arranged based on the size of the subject's foot. Similarly, electrode placement on the insole may be selected based on the area of the subject to be treated. The insole 2060 may be placed in one of treatment devices described herein. As shown, a treatment device 2070 is designed for treatment of the left foot and a treatment device 2080 is designed for treatment of the right foot. The intensity settings and waveforms applied using the treatment devices 2070 and 2080 may be the same or may be different. A cartridge 2090 may be coupled to one or both of the treatment devices 2070, 2080 to provide fluid to them. Each of treatment devices 2070 and 2080 may be controlled, for example, using a remote 2050.

### Example 3

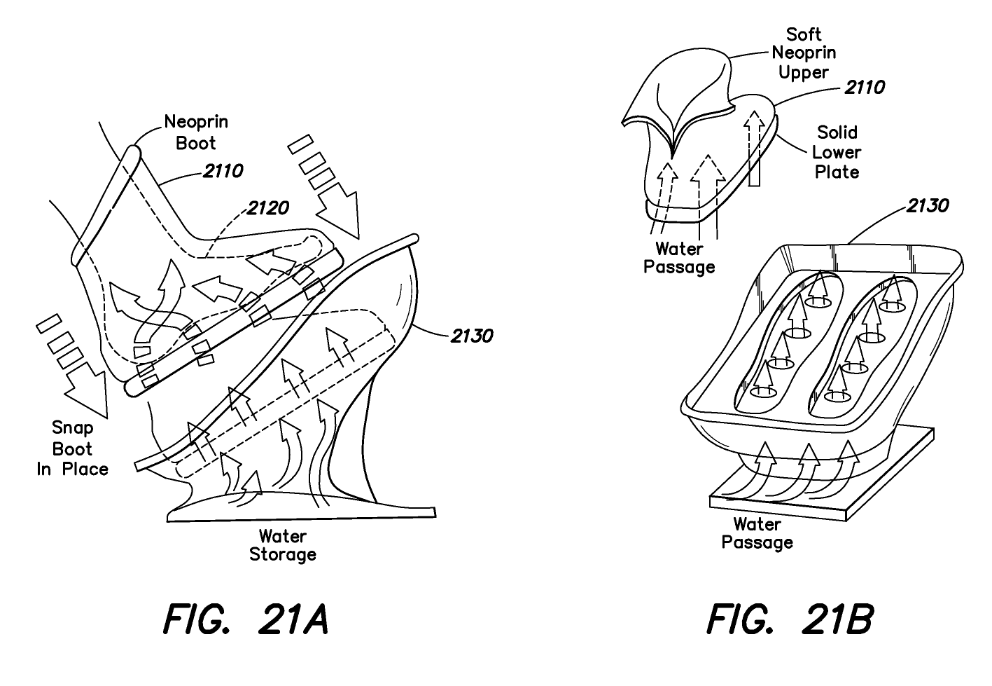

Referring to FIGS. 21A and 21B, a boot 2110 is shown that is constructed and arranged to receive a user's foot 2120 for treatment. The boot 2110 may be designed for placement into a treatment device 2130, e.g., a footbath, such that treatment may be provided using one or more waveforms such as, for example, those described herein. The treatment device 2130 may be controlled remotely using a hand held device.

The boot 2110 is constructed and arranged such that fluid from the treatment device 2130 may enter into the boot 2110 through lower surfaces of the boot 2110. For example, the lower portion of the upper may be fluid permeable, the sole of the boot 2110 may be fluid permeable or both may be fluid permeable. The sole may include integrated electrodes such that placement of the boot 2110 in the treatment device 2130 provide for electrical coupling to facilitate delivery of current to the foot 2120.

### Example 4

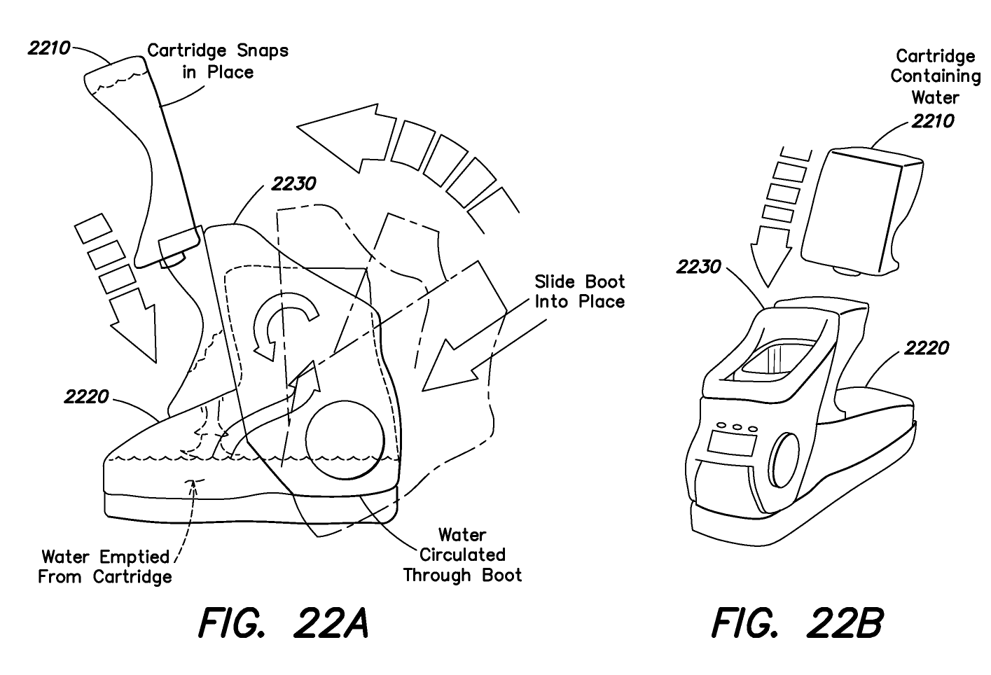

Referring to FIGS. 22A and 22B, a cartridge 2210 is shown that may be configured to mate or couple to a boot 2220 or other device. The cartridge 2210 may contain fluid and permit easier application of fluid by a user than the large volume of fluid used in a footbath. In operation (see FIG. 22B), the cartridge 2210 may be inserted into a guide or sleeve 2230 of the boot 2220 such that application of force to seat the cartridge 2210 results in rupture of a seal or gasket of the cartridge 2210 and delivery of its contents to the boot 2220. In other examples (see FIG. 22A), the cartridge 2210 may be inserted into the sleeve 2230 and movement of the sleeve 2230 to second position may rupture the seal or gasket of the cartridge 2210 to deliver its contents. A fluid conduit may connect the cartridge sleeve 2230 to the internal portions of the boot 2220 such that the fluid can be provided to one or substantially all areas of the foot.

### Example 5

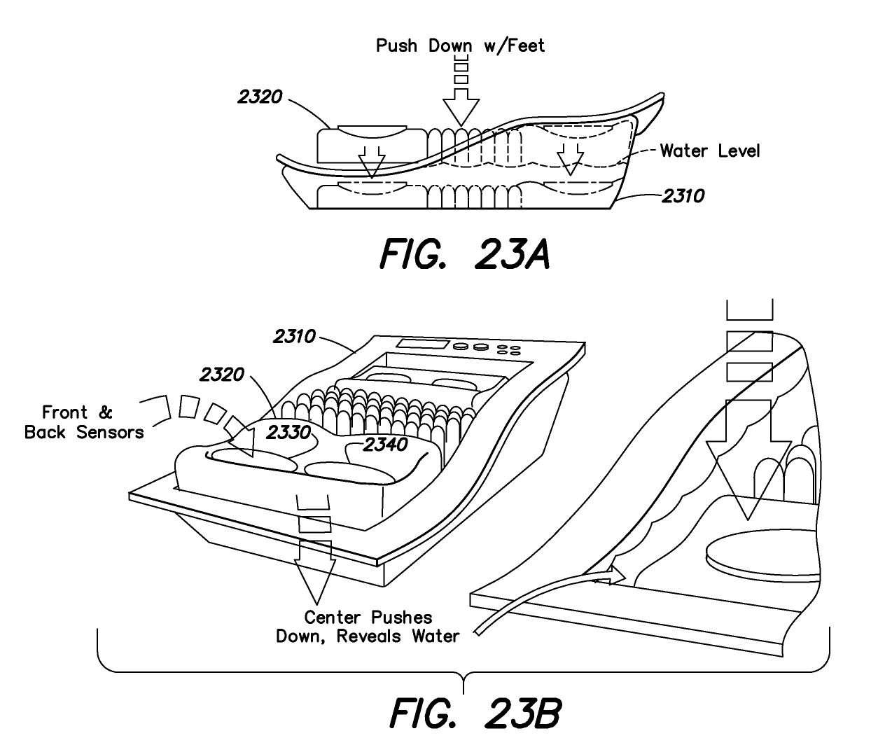

Referring to FIGS. 23A and 23B, a schematic of a device where direct pressure applied would enable compression of the material such that the feet would then come in contact with the water as it is displaced is shown. In the illustration shown in FIGS. 23A and 23B, the device includes a housing 2310, an insert 2320, and electrodes 2330 and 2340. Direct pressure applied to the insert 2320 can enable compression of the material of the insert 2320 such that the feet would then come in contact with the water as it is displaced from above. For example, a user may stand to provide a downward gravitation force such that fluid is pushed out of the material of the insert 2320 to wet the feet. The electrodes 2330 and 2340 can provide treatment as described herein.

### Example 6

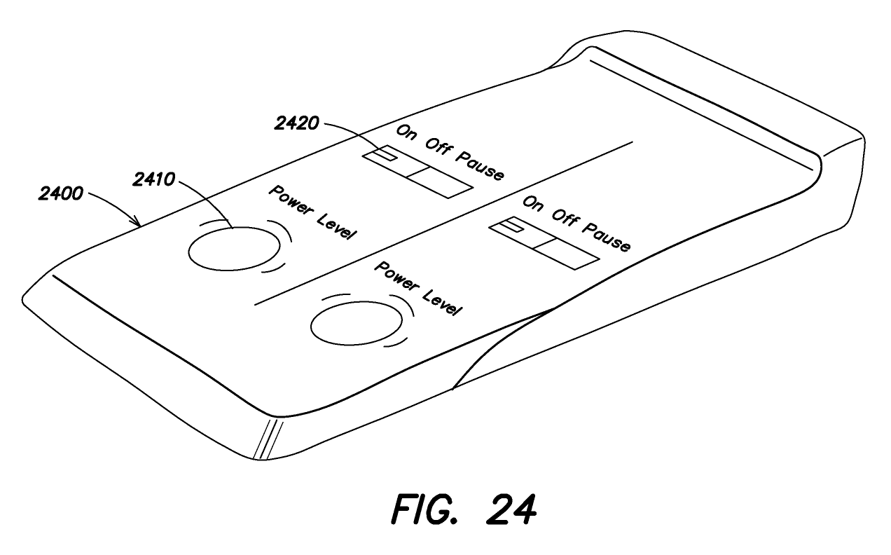

A remote 2400 is shown in FIG. 24. The remote 2400 may take the form of a wired or wireless remote control, as shown for example in many of the figures attached hereto, and may include user selected buttons to facilitate treatment. For example, the remote may include one or more buttons or features to enable a user to select and/or scroll through menu options on a display of a waterbath.

In the embodiment shown in FIG. 24, the remote is configured to operate two treatment devices. On the left side of the remote 2400, a button 2410 may be depressed to control the intensity of treatment. For example, two or more settings may be programmed into the remote 2400, e.g., low, medium and high, such that depression of the button can toggle between the intensity settings. A slideable switch 2420 may also be included so that, for example, a user can turn on treatment, turn off treatment or pause treatment. Similar controls may be present on the right side of the remote 2400 to facilitate use of a single remote 2400 to control treatment using separate devices, e.g., such as devices 2070 and 2080 of FIG. 20B.

In some examples, the size and shape of the remote may be selected to permit for (1) comfortable positioning in the hand (2) easy activation of the device from a seated position, sufficient cord length to allow for positioning of the remote on a waist strap or the like. That is, user features may be included to increase the overall usability of the remote and/or to enhance the aesthetic appearance of the remote.

### Example 7

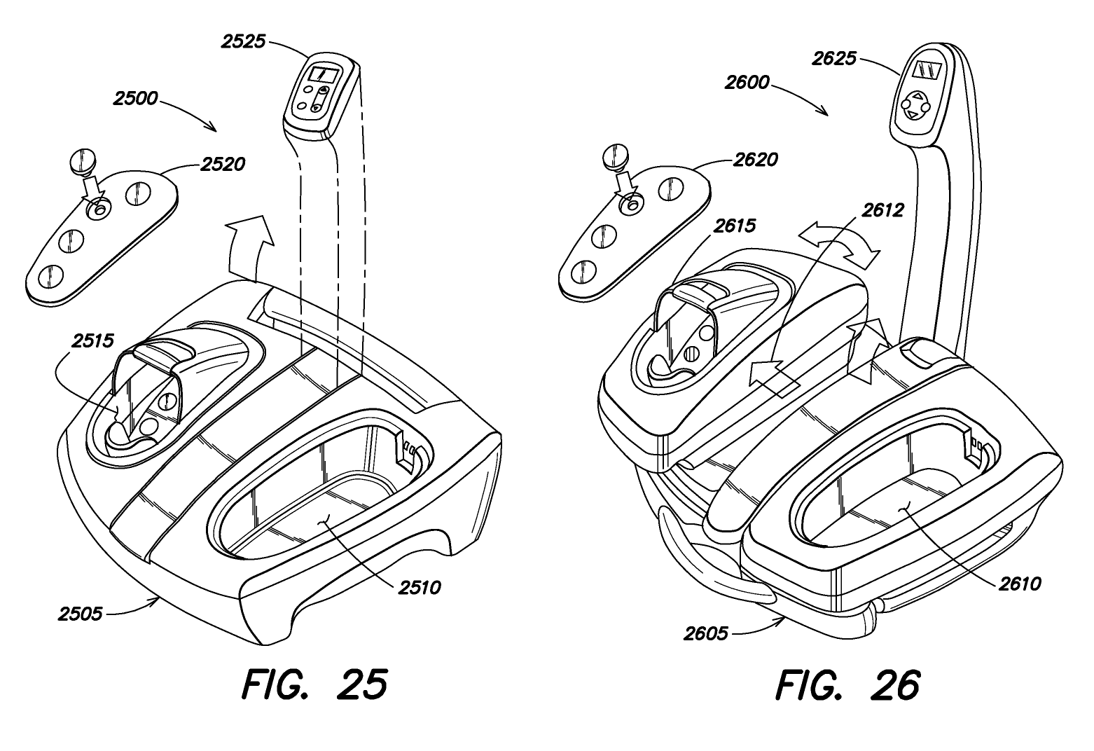

A waterbath having multiple receptacles configured to receive treatment compartments can be used to provide the treatment methods described herein. Referring to FIG. 25, a waterbath 2500 includes a housing 2505 having two receptacles such as receptacle 2510 each configured to receive a treatment compartment, such as treatment compartment 2515. The treatment compartment 2515 can be used with an insole 2520 such as the insole described in reference to FIG. 15, for example, or the insole 2520 shown in FIG. 25. The waterbath 2500 includes a controller 2525 that is operative to receive user input or selections and to implement a treatment method based on the user selections. The waterbath 2500 can be used to treat any one or more disorders described herein and can desirably be used to treat loss of sensation in the foot, to prevent loss (or further loss) of sensation of the foot, or other disorders of the foot.

In use, a treatment compartment would be introduced into each receptacle and coupled to the receptacle through a suitable electrical connector on each of the receptacle and the compartment. A fluid and optionally other species would be introduced into each of the compartments. The user would then enter treatment parameters using the display/controller 2525, and treatment would be initiated. When treatment is finished, the user can then remove each of the compartments from the waterbath 2500 and empty the contents in a sink.

### Example 8

A waterbath having multiple receptacles configured to receive treatment compartments can be used to provide the treatment methods described herein. Referring to FIG. 26, a waterbath 2600 includes a housing 2605 having two receptacles, such as receptacle 2610, each configured to receive a treatment compartment, such as treatment compartment 2615. The position of the receptacles can be adjusted laterally, as shown by arrow 2612, to provide enhanced subject comfort. Each of the receptacles can be adjusted independently of the other receptacle. The treatment compartment 2615 can be used with an insole 2620 such as the insole described in reference to FIG. 15, for example. The waterbath 2600 includes a display/controller 2625 that is operative to receive user input or selections and to implement a treatment method based on the user selections. The waterbath 2600 can be used to treat any one or more disorders described herein and can desirably be used to treat loss of sensation in the foot, to prevent loss (or further loss) of sensation of the foot, or other disorders of the foot.

In use, a user would position the receptacles at a desired spacing where the user is comfortable. A treatment compartment would be introduced into each receptacle and coupled to the receptacle through a suitable electrical connector on each of the receptacle and the compartment. A fluid and optionally other species would be introduced into each of the compartments. The user would then enter treatment parameters using the display/controller 2625, and treatment would be initiated. When treatment is finished, the user can then remove each of the compartments from the waterbath 2600, using the handle on each of the compartments, and empty the contents in a sink.

### Example 9

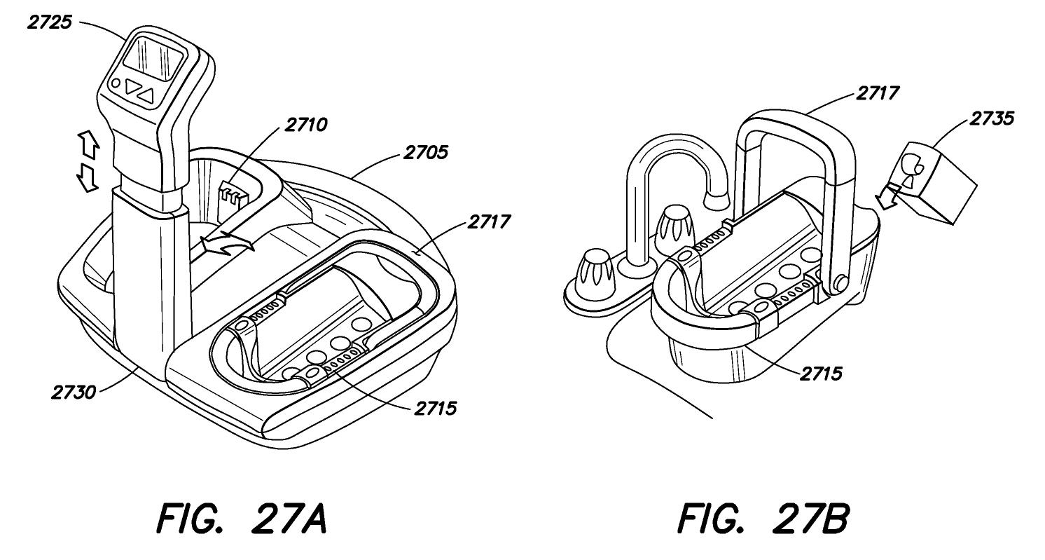

A waterbath having multiple receptacles configured to receive treatment compartments can be used to provide the treatment methods described herein. Referring to FIGS. 27A and 27B, a waterbath 2700 includes a housing 2705 having two receptacles, such as receptacle 2710, each configured to receive a treatment compartment, such as treatment compartment 2715. The treatment compartment 2715 can be used with an insole such as the insole described in reference to FIG. 15, for example, or can be used without an insole. The waterbath 2700 includes a removable remote 2725 that is operative to receive user input or selections and to send a suitable signal to a controller to implement a treatment method based on the user selections. The waterbath 2700 can be used to treat any one or more disorders described herein and can desirably be used to treat loss of sensation in the foot, to prevent loss (or further loss) of sensation of the foot, or other disorders of the foot.

In use, a treatment compartment would be introduced into each receptacle and coupled to the receptacle through a suitable electrical connector on each of the receptacle and the compartment. A fluid and optionally other species, such as those in a container 2735, can be introduced into each of the compartments. The user can then enter treatment parameters using the remote 2725. For example, the remote 2725 can be removed from the waterbath 2700 and treatment parameters can be selected by a user. The remote 2725 can then be docked or engaged with an arm 2730 of the waterbath 2700 to transfer the selected treatment parameters and to initiate treatment. When treatment is finished, the user can then remove each of the compartments from the waterbath 2700, using the handle on each of the compartments, and empty the contents in a sink. In FIG. 27B, the handle 2717 of the compartment 2715 is shown as attaching to the top sides of the compartment 2715 at a midpoint. As described herein, however, the handle may be attached in other positions.

### Example 10

An illustrative testing protocol is described below. The purpose of this testing was to collect operational data on the twin-peak monophasic output of a Rich-Mar Theratouch 4.7 device and an Acticare TSE device. Data was collected and compared on a single sample of each device under various load and output amplitude conditions as detailed below.

A TheraTouch 4.7 electrical stimulation device with cable set (2) (S/N 1113062541) and an Acticare TSE electrical stimulation device with cable set (Y cable to drive 2 pairs of electrodes, built in house) (S/N A000030009847) were each tested. Also used in the tests were: an oscilloscope with (2) 10× probes (Wrx1032), 5000, 500, 50 ohm resistive loads (measured values: 4960, 497, 50.4 ohms), resistor assortment 10-10 k ohms for current sampling, a digital voltmeter (Wrx1054) and a twin compartment footbath with four electrodes. A solution of water including 0.6% hydrogen peroxide was also used.

This testing was performed under environmental conditions that reflect the typical office environment of nominally 25° C. and under nominal line voltage conditions of 120 VAC/60 Hz. The output variables consist of data and observations recorded during testing.

The test protocol called for the following tests to be performed. All collected data, observations, deviations, and comments were recorded. Oscilloscope images were time stamped by the scope when captured.

The Theratouch device was set to generate a twin-peak monophasic output with a 50 uS pulse, 100 uS interpulse period, and 120 Hz waveform frequency. The output was loaded with the following resistive loads: open, 5000, 500, 50 ohms (+/−10%). The output amplitude was adjusted to a screen reading of 25 mA and 50 mA. For the 500 ohm load at 50 mA the following was recorded (pulse periods were measured at 50%, rise and fall times=10%-90%): mean voltage pulse amplitude for each pulse, pulse width for each pulse, interpulse period, and waveform frequency.

For each output load and amplitude combination the following was collected: oscilloscope waveform, and mean voltage pulse amplitude for each pulse.

Next, the Theratouch device was set to generate a twin-peak monophasic output with a 50 uS pulse, 100 uS interpulse period, and 100 Hz waveform frequency. The device was connected to two 4″ square electrodes in one of the compartments of the foot bath, connecting the negative lead to the electrode at the toe. A 1000 ohm sampling resistor was connected in a lead from the device. The device output voltage and voltage across the sampling resistor with the oscilloscope were monitored. When collecting data, the sampling resistor was adjusted so that the voltage drop across the resistor is less than 20% of output voltage. A water solution of 0.6% H₂O₂ was added to the foot bath along with one foot of a human volunteer test subject.

The device was started and the amplitude was increased to a maximum comfort level not to exceed 50 mA as displayed on screen. The current sample resistor was adjusted if required (with the device off). The new value was recorded if changed. The oscilloscope display was captured. The mean output voltage and current resistor voltage pulse amplitude for each pulse was recorded. The device was then shut down.

Using the Acticare device, the following protocol was implemented: the Acticare device was set to generate a twin-peak monophasic output with a 50 uS pulse, 100 uS interpulse period, and 100 Hz waveform frequency; the output was loaded with the following resistive loads: open, 5000, 500, 50 ohms (+/−10%); the output amplitude was adjusted to a screen reading of 14% and 28%; for the 500 ohm load at 28% the following was recorded (pulse periods to be measured at 50%, rise and fall times=10%-90%): mean voltage pulse amplitude for each pulse, pulse width for each pulse, interpulse period, and waveform frequency. For each output load and amplitude combination the following was collected: oscilloscope waveform, and mean voltage pulse amplitude for each pulse.

Next, the Acticare device was set to generate a twin-peak monophasic output with a 50 uS pulse, 100 uS interpulse period, and 100 Hz waveform frequency and the following protocol was used: the device was connected to two 4″ square electrodes in one compartment of the foot bath, connecting the negative lead to the electrode at the toe. The foot bath type used and electrode details were recorded in the lab notebook. A 1000 ohm sampling resistor was connected in a lead from the device. The device output voltage and voltage across the sampling resistor were monitored with the oscilloscope. A water solution of 0.6% H₂O₂ was added to the foot bath along with one foot of a human volunteer test subject. The device was started and the amplitude increased to a maximum comfort level not to exceed 28% as displayed on screen. The output amplitude setting was recorded. The current sample resistor was adjusted if required (with the device off). The new value was recorded if changed. The oscilloscope display output was captured. Mean output voltage and current resistor voltage pulse amplitude were recorded for each pulse. The device was shut down and a second set of electrodes were connected in their own foot bath compartment in parallel with the first; adding a water solution of 0.6% H₂O₂ and a second foot of the human volunteer test subject to the second foot bath. Again, the device was started and the amplitude increased to a maximum comfort level not to exceed 28% as displayed on screen. The output amplitude setting was recorded. The current sample resistor was adjusted if required (with the device off). The new value was recorded if changed. The oscilloscope display output was captured. The mean output voltage and current resistor voltage pulse amplitude for each pulse was recorded. The device was shut down.

In the next measurements, the following protocol was implemented: both the Theratouch and Acticare units were set to generate the waveforms used in the previous sections and with the same current sampling and output monitoring network. The following sequence of data collection and sensation testing steps were repeated on four individuals. For each individual data was recorded for one foot and for both feet in parallel with the Theratouch device. For each individual data was recorded for one foot and for both feet in parallel with the Acticare device. For each test the output intensity was increased to the following points and an image of the oscilloscope screen was captured: point of 1^{st} sensation, point of maximum comfort, 40% intensity for the Acticare device, 50 mA intensity for the Theratouch device; and for the 40% and 50 mA levels the RMS voltages as calculated by the oscilloscope were recorded.

To characterize the linearity of the devices over their output range peak, rms, and area under the curve data for both devices over their output range while driving a 500 ohm load were collected.

The results of the above measurements are now discussed. For the Theratouch device, oscilloscope images were captured at each recorded setting and loading. With a 500 ohm load and 50 mA setting the following values were recorded:

| 500 ohm | Pulse 1 | Pulse 2 |
|---|---|---|
| Voltage | 23 | 23 |

| | |
|---|---|
| Expected Voltage | 50 mA * 500 ohm = 25 V |

| | | |
|---|---|---|
| Pulse Width (uS) | 47 | 47 |

| | |
|---|---|
| Interpulse (uS) | 99 |
| Waveform Freq. | 120.4 Hz |

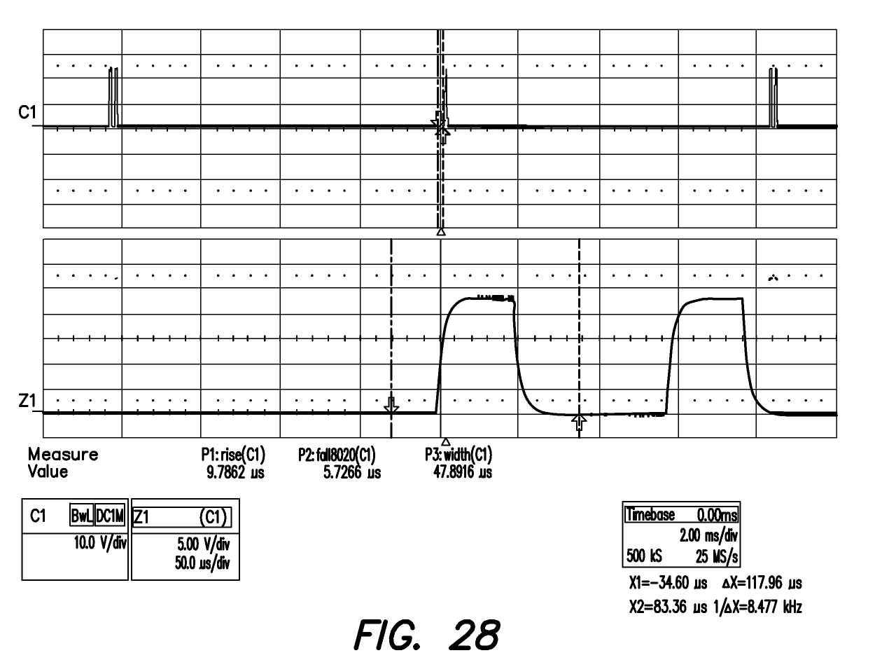

This table shows that the Theratouch device generates the voltage peak and timing expected per the user's guide. This assumes that the displayed amplitude value expects a 500 ohm load to be used. The peak voltage was approximately 10% below the calculated value. An oscilloscope trace that was captured is shown in FIG. 28.

As noted above the Theratouch device was loaded down with a sequence of resistive loads and waveform voltage value was recorded at panel settings of 25 mA and 50 mA. In this case the mean value was taken to be the value at the midpoint of the pulse.

Theratouch Voltage

| | |
|---|---|
| 25 mA Setting | 50 mA Setting |

| Load | Pulse 1 | Pulse 2 | Pulse 1 | Pulse 2 |
|---|---|---|---|---|
| Open | 13.3 | 13 | 26.6 | 26.7 |
| 5000 | 13 | 13 | 26.3 | 26.2 |
| 500 | 11.6 | 11.5 | 23 | 22.9 |
| 50 | 4.6 | 4.6 | 8.9 | 8.9 |

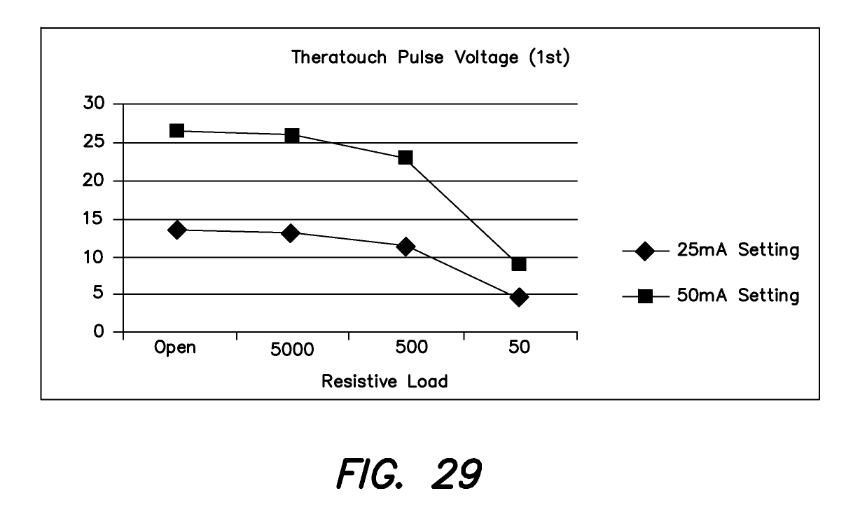

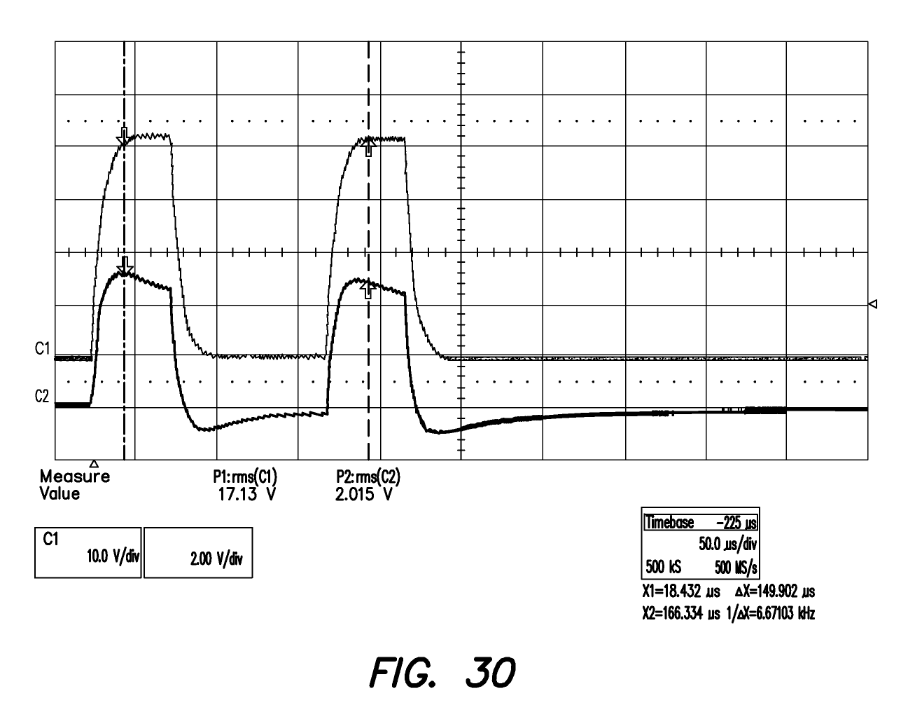

As can be seen in the table above and the chart shown in FIG. 29, the output impedance of the device limits the voltage delivered as the load impedance drops. Note that this chart shows that even though a current value is shown on the display as the output is adjusted, the unit supplies a fixed voltage within the limits of the output impedance.

The Theratouch device was set up as described above to generate a twin-peak monophasic output with a 50 uS pulse, 100 uS interpulse period, and 100 Hz waveform frequency. Oscilloscope images were captured at each recorded setting. A Sterilite Caddy, 1584, and Mettler 4″ square electrodes, part number 2002, were used with 2 liters of tap water and 500 ml of 3% H₂O₂ in each foot compartment. The device output voltage was monitored with channel 1 of the oscilloscope and the voltage across a current sample resistor connected in series with the negative lead was monitored with channel 2. The sample resistor was changed from the 1000 ohm value initially called for in the test plan to a 50 ohm value to minimize the effect on the output voltage.

With a one foot load, the maximum level the subject could comfortably tolerate was 90 mA. At this setting the following values were recorded: note that since both pulses were very similar only the readings for the first pulse are shown. A current scaling factor of the square root of (500 uS/8333 uS) is due to only a portion of the full 120 Hz waveform being displayed on the oscilloscope screen (see FIG. 30).

| | |
|---|---|
| Theratouch | 90 mA Setting |
| Subject 1 | One Foot |
| Voltage Ch1 | 42 V |
| Expected Voltage | 90 mA * 500 ohm = 45 V |
| Current Ch2 (rms) | 2.0 V/50 ohm * √(500 uS/8333 uS) = 9.8 mA |

Turning now to the data acquired with the Acticare device, the Acticare device was set up as noted above to generate a twin-peak monophasic output with a 50 uS pulse, 100 uS interpulse period, and 100 Hz waveform frequency. Oscilloscope images were captured at each recorded setting and loading. Due to the large tilt in the Acticare output under load, both the peak and the minimum pulse voltage were recorded.

With a 500 ohm load and 28% setting the following values were recorded:

| 500 ohm | Pulse 1 peak | Pulse 1 min | Pulse 2 peak | Pulse 2 min |
|---|---|---|---|---|
| Voltage | 21.5 | 13.0 | 19.6 | 13.0 |

| Expected | 90 V * 28% = 25.2 V | |
|---|---|---|
| Voltage | | |
| Pulse Width | 49.9 | 49.9 |
| (uS) | | |

| | |
|---|---|
| Interpulse (uS) | 100 |
| Waveform | 100 |
| Freq. | |

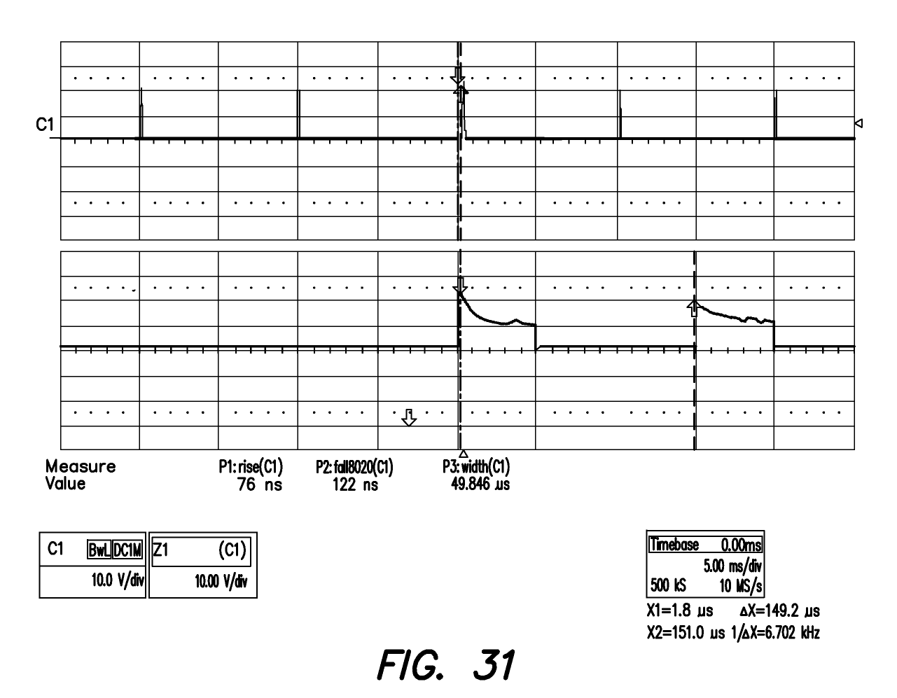

This table shows that the Acticare device generated the voltage peak and timing expected per the user's guide. The peak voltage was approximately 20% below the calculated value. What was not expected was the droop seen across the pulse width. This observation led to the additional testing detailed below to show that the droop was predictable. Review of the manufacturer's patent concerning the device also supported the observed results. FIG. 31 shows one of the oscilloscope images.

As noted above, the Acticare device was loaded down with a sequence of resistive loads and waveform voltage value was recorded at panel settings of 14% and 28%. In this case the mean value was taken to be the value at the mean of the peak and minimum waveform voltages.

Acticare Mean Voltage ((pk + min)/2)

| | |
|---|---|
| 14% Setting | 28% Setting |

| Load | Pulse 1 | Pulse 2 | Pulse 1 | Pulse 2 |
|---|---|---|---|---|
| Open | 9 | 8.85 | 23 | 23 |
| 5000 | 8.7 | 8.45 | 22.1 | 21.1 |
| 500 | 6.75 | 6.25 | 17.7 | 16.6 |
| 50 | 4.25 | 4.1 | 9.5 | 9 |

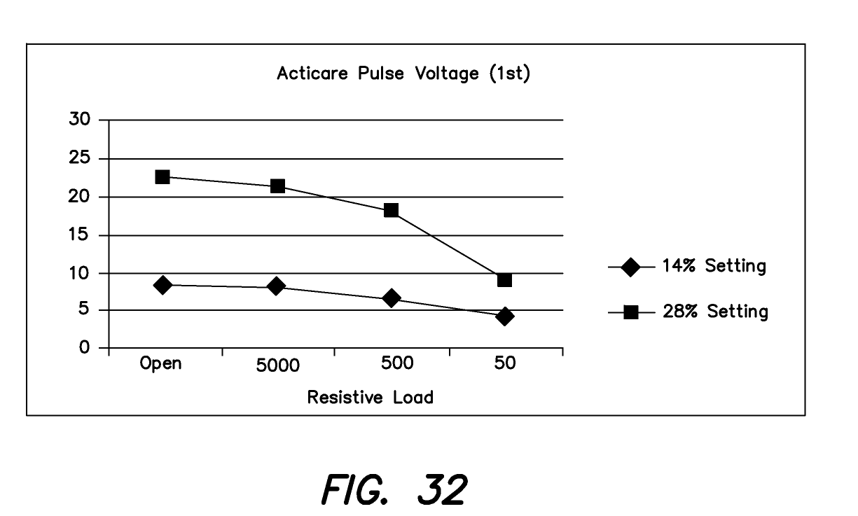

As can be seen in the table above and the chart in FIG. 32, the output impedance of the device limits the voltage delivered as the load impedance drops.

The Acticare device was set up as noted above to generate a twin-peak monophasic output with a 50 uS pulse, 100 uS interpulse period, and 100 Hz waveform frequency. Oscilloscope images were captured at each recorded setting. The device output voltage and current was monitored.

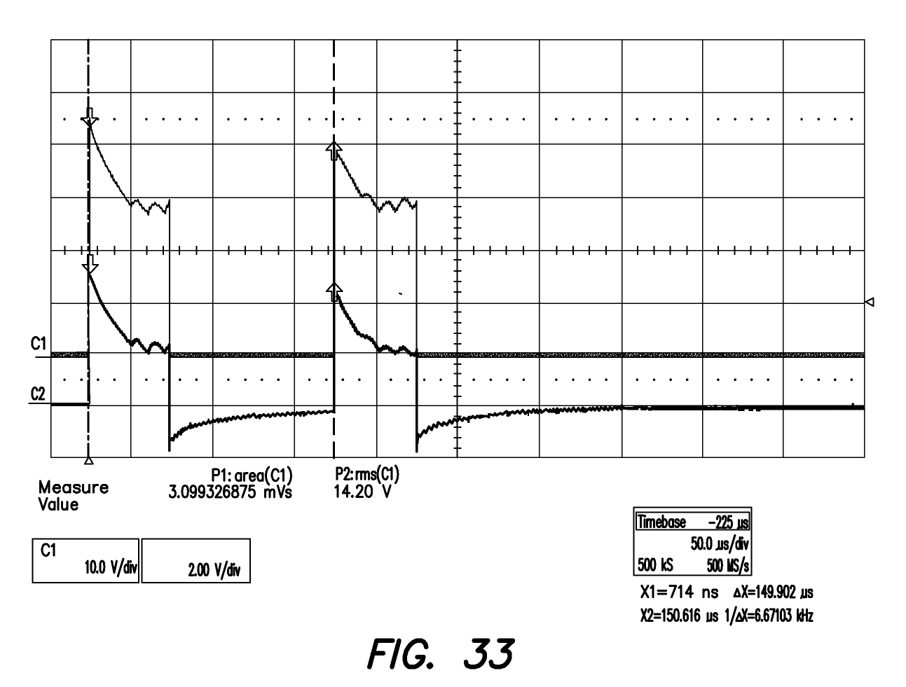

With a one foot load the maximum level the subject could comfortably tolerate was 50%. At this setting the following values were recorded and the image shown in FIG. 33 was recorded. Note that since both pulses were very similar only the readings for the first pulse are shown.

| | |
|---|---|
| Acticare | 50% Setting |
| Subject 1 | One Foot |
| Voltage Ch1 | Vpk: 44 V, Vmin: 30 V, Vmean: 37 V |
| Expected Voltage | 90 V * 50% = 45 V |
| Current Ch2 | 1.3 V/50 ohm * √(500 uS/10000 uS) = 5.8 mA |

Again, the peak voltage follows the formula expected from the user's guide but the signal has a significant droop that is now better understood.

The following measurements were performed to provide a set of data for determining what criteria could be used to compare the expected effect of the devices. Each subject was taken to the point of first sensation and to the maximum level they felt they could handle for thirty minutes. This was done with one foot first and then with both feet with the electrodes connected in parallel. Data and oscilloscope images were captured at each test point and at our proposed limit values of 50 mA on the Theratouch device and 40% on the Acticare device.

The test plan only called for the RMS values to be collected at the 50 mA and 40% values but as testing progressed it became clear that this might be the best gauge for comparing devices so, after subject 1, RMS values were collected for all readings. For the following table of comparisons some of the current values for subject 1 were calculated from the applied voltage values collected. The table below shows the results of testing four subjects on the two devices. The average value for the measured current from the Theratouch device was 16% higher than the Acticare device with a coefficient of variation of 32%.

| | | | Acticare | | Theratouch | | |
|---|---|---|---|---|---|---|---|
| | | | Vrms | Irms | Vrms | Irms | TT/AC |
| Subject | Sense | Feet | meas. | (mA) | meas. | (mA) | Ratio |
| 1 | 1st | one | 0.7 | 3.13 | 1.3 | 6.37 | 2.03 |
| 1 | Max | one | 1.3 | 5.81 | 2 | 9.80 | 1.69 |
| 1 | 1st | two | 1.46 | 6.53 | 1.6 | 7.84 | 1.20 |
| 1 | Max | two | 2.45 | 10.96 | 2.6 | 12.74 | 1.16 |
| 2 | 1st | one | 0.67 | 3.00 | 0.621 | 3.04 | 1.02 |
| 2 | Max | one | 1.15 | 5.14 | 0.725 | 3.55 | 0.69 |
| 2 | 1st | two | 0.97 | 4.34 | 1.07 | 5.24 | 1.21 |
| 2 | Max | two | 1.3 | 5.81 | 1.13 | 5.54 | 0.95 |
| 3 | 1st | one | 0.99 | 4.43 | 0.93 | 4.56 | 1.03 |
| 3 | Max | one | 2.19 | 9.79 | 1.6 | 7.84 | 0.80 |
| 3 | 1st | two | 1.9 | 8.50 | 1.4 | 6.86 | 0.81 |
| 3 | Max | two | 2.9 | 12.97 | 2.1 | 10.29 | 0.79 |
| 4 | 1st | one | 0.88 | 3.94 | 1.36 | 6.66 | 1.69 |
| 4 | Max | one | 1.72 | 7.69 | 1.854 | 9.08 | 1.18 |
| 4 | 1st | two | 2.15 | 9.62 | 2.37 | 11.61 | 1.21 |
| 4 | Max | two | 3.6 | 16.10 | 3.37 | 16.51 | 1.03 |
| | | | | | | Mean | 1.16 |
| | | | | | | % CV | 32% |

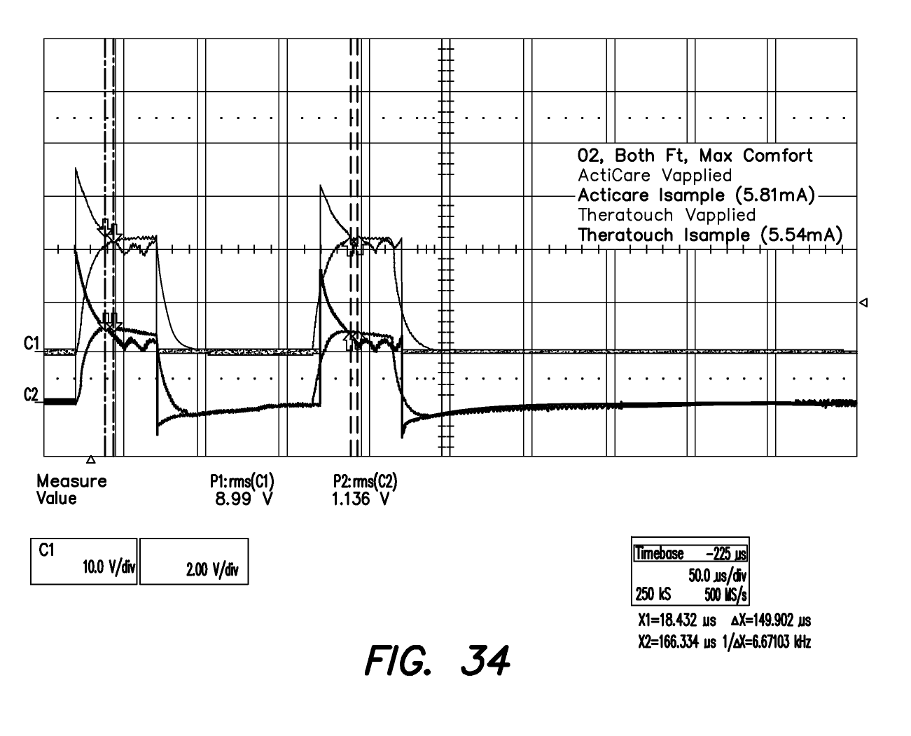

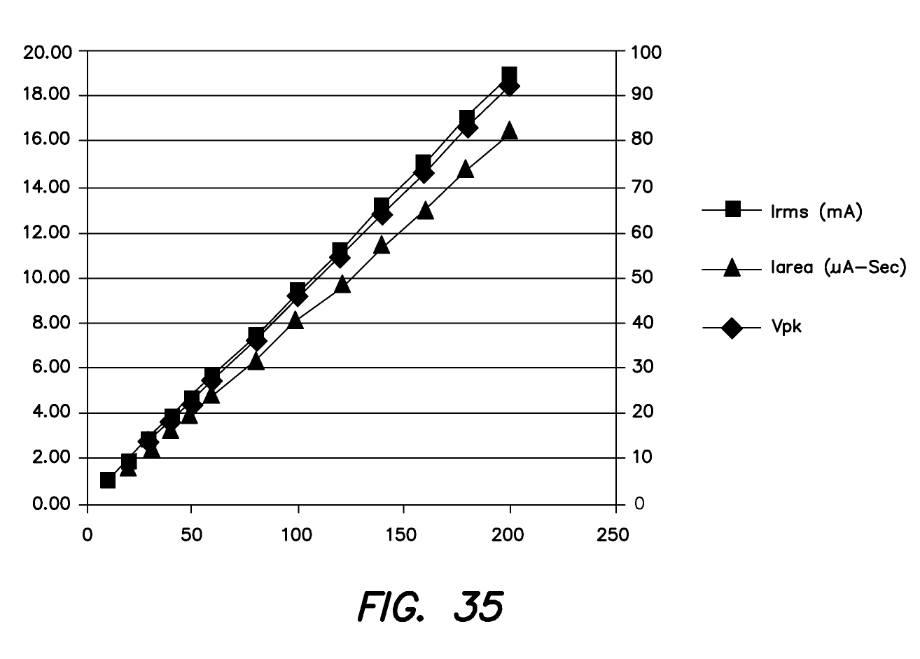

As a visual example of how the output of the two devices compare, shown in FIG. 34 is an overlay of the oscilloscope traces for subject 2. These images were captured with both feet driven to the maximum comfort level. This is the last line shown in the table above for subject 2. As detailed above, traces were collected of the applied voltage waveform and the voltage across a 50 ohm current sampling resistor in series with the negative output line. The RMS current value was calculated by the oscilloscope and then adjusted for the difference between the displayed portion of the waveform (500 uS) and the total period of waveform (10 mS for the Acticare device and 8.33 mS for the Theratouch device). The Acticare waveforms are shown in green and blue, and the Theratouch waveforms are shown in yellow and magenta as listed in the legend.

As can be seen the bulk of the waveforms overlap almost exactly, with the Acticare device showing much faster rise and fall times as well as a droop after the initial voltage peak. If the treatment limits are adjusted to account for this droop, comparing RMS current values this waveform should be expected to be just as effective as the Theratouch device.

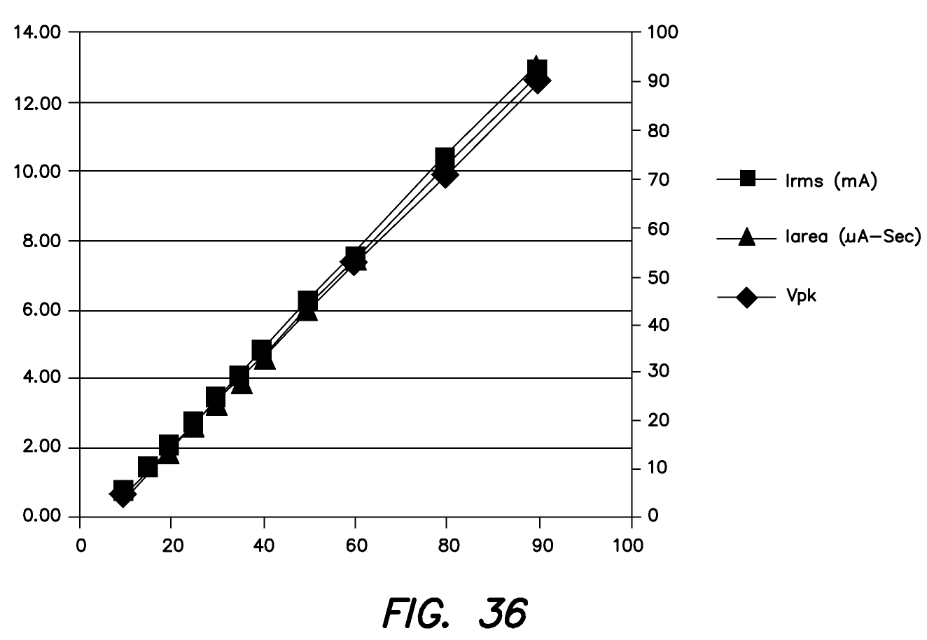

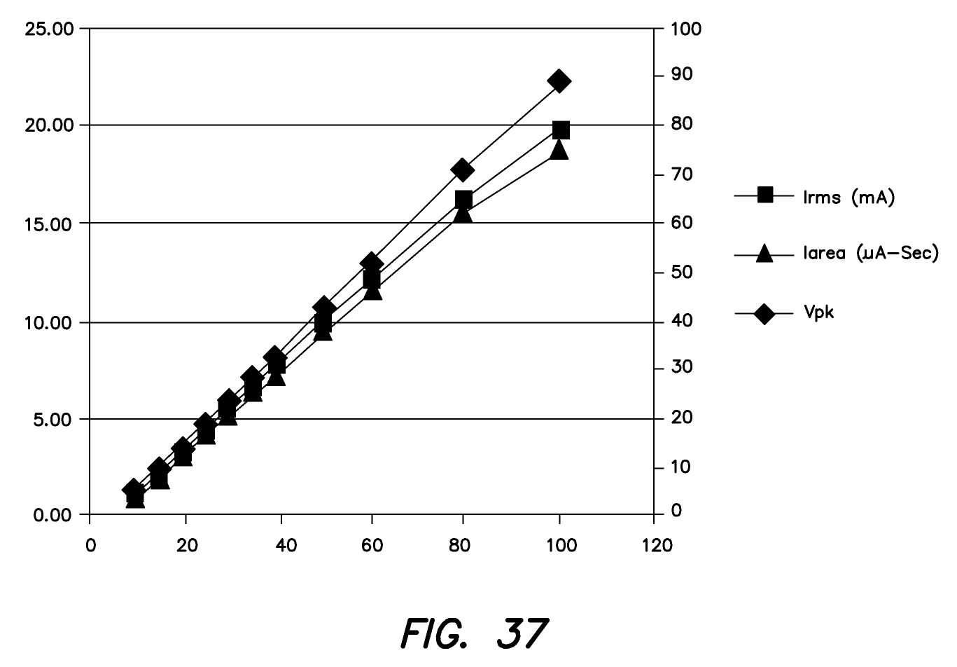

Shown below are data collected across the working range of the two devices into a 500 ohm load to demonstrate linearity and to determine comparable settings. In addition, a load sweep was performed on the Acticare device with a 250 ohm load to confirm that its output was still linear with a heavier load. A limit of 50 mA had been proposed for use with the Theratouch device. This results in a 4.62 mA RMS load current. Similar RMS load current is generated by the Acticare unit at a setting of 40%, at 4.83 mA this unit generates a current less than 5% higher. This value could be more closely matched but it is desirable to use a “whole” number value for ease of use. Graphs of data from the Theratouch device and the Actic are device are shown in FIGS. 35 and 36, respectively. A graph of data from the Acticare device at the 250 ohm load is shown in FIG. 37.

Theratouch 4.7
500 ohm load sweep

| Output | | Vrms | | | Varea | Iarea |
|---|---|---|---|---|---|---|
| Setting | | meas. | Vrms | Irms | (mV- | (uA- |
| (mA) | Vpk | (500 uS) | (8.3 mS) | (mA) | Sec) | Sec) |
| 200 | 92 | 38.5 | 9.43 | 18.86 | 8.2 | 16.4 |
| 180 | 83 | 34.7 | 8.50 | 17.00 | 7.4 | 14.8 |
| 160 | 73 | 30.7 | 7.52 | 15.04 | 6.5 | 13 |
| 140 | 64 | 26.9 | 6.59 | 13.18 | 5.7 | 11.4 |
| 120 | 55 | 22.9 | 5.61 | 11.22 | 4.8 | 9.6 |
| 100 | 46 | 19.1 | 4.68 | 9.36 | 4.1 | 8.2 |
| 80 | 36.3 | 15.2 | 3.72 | 7.45 | 3.24 | 6.48 |
| 60 | 27.5 | 11.5 | 2.82 | 5.63 | 2.43 | 4.86 |
| 50 | 22 | 9.44 | 2.31 | 4.62 | 2 | 4 |
| 40 | 18.6 | 7.75 | 1.90 | 3.80 | 1.68 | 3.36 |
| 30 | 14 | 5.84 | 1.43 | 2.86 | 1.27 | 2.54 |
| 20 | 9.22 | 3.84 | 0.94 | 1.88 | 0.85 | 1.7 |
| 10 | 4.6 | 1.93 | 0.47 | 0.95 | 0.43 | 0.86 |

ActiCare TSE
500 ohm load sweep

| Output | | Vrms | | | Varea | Iarea |
|---|---|---|---|---|---|---|
| Setting | | meas. | Vrms | Irms | (mV- | (uA- |
| (%) | Vpk | (500 uS) | (10 mS) | (mA) | Sec) | Sec) |
| 100 | 90 | 29.1 | 6.51 | 13.01 | 6.4 | 12.8 |
| 80 | 71 | 23.2 | 5.19 | 10.38 | 5.1 | 10.2 |
| 60 | 52.5 | 16.9 | 3.78 | 7.56 | 3.7 | 7.4 |
| 50 | 43.3 | 13.9 | 3.11 | 6.22 | 3 | 6 |
| 40 | 33.1 | 10.8 | 2.41 | 4.83 | 2.3 | 4.6 |
| 35 | 28.7 | 9.1 | 2.03 | 4.07 | 2 | 4 |
| 30 | 24 | 7.7 | 1.72 | 3.44 | 1.6 | 3.2 |
| 25 | 19.2 | 6.12 | 1.37 | 2.74 | 1.3 | 2.6 |
| 20 | 14.3 | 4.55 | 1.02 | 2.03 | 0.93 | 1.86 |
| 15 | 9.5 | 3.03 | 0.68 | 1.36 | 0.7 | 1.4 |
| 10 | 4.7 | 1.55 | 0.35 | 0.69 | 0.35 | 0.7 |

ActiCare TSE
250 ohm load sweep

| Output | | Vrms | | | Varea | Iarea |
|---|---|---|---|---|---|---|
| Setting | | meas. | Vrms | Irms | (mV- | (uA- |
| (%) | Vpk | (500 uS) | (10 mS) | (mA) | Sec) | Sec) |
| 100 | 89 | 22.2 | 4.96 | 19.86 | 4.7 | 18.8 |
| 80 | 71 | 18 | 4.02 | 16.10 | 3.9 | 15.6 |
| 60 | 51.8 | 13.5 | 3.02 | 12.07 | 2.9 | 11.6 |
| 50 | 42.5 | 11 | 2.46 | 9.84 | 2.36 | 9.44 |
| 40 | 32.5 | 8.5 | 1.90 | 7.60 | 1.8 | 7.2 |
| 35 | 28.3 | 7.4 | 1.65 | 6.62 | 1.6 | 6.4 |
| 30 | 23.5 | 6.15 | 1.38 | 5.50 | 1.3 | 5.2 |
| 25 | 18.9 | 4.89 | 1.09 | 4.37 | 1.05 | 4.2 |
| 20 | 14 | 3.64 | 0.81 | 3.26 | 0.77 | 3.08 |
| 15 | 9.2 | 2.36 | 0.53 | 2.11 | 0.49 | 1.96 |
| 10 | 4.5 | 1.18 | 0.26 | 1.06 | 0.24 | 0.96 |

The test plan called for the mean value to be recorded. Due to the tilt of the pulse top, the peak and minimum pulse voltage were recorded and the mean value of these two readings was calculated. The linearity check on the Acticare unit was not in the original test plan. It was added to confirm that the waveshape seen was not due to active per pulse current limiting by the Acticare device.

Based on the collected data, both the Acticare TSE and Theratouch device can be used to provide a similar stimulus effect. The Acticare device may be simpler for subjects to use in a home setting.

### Example 11

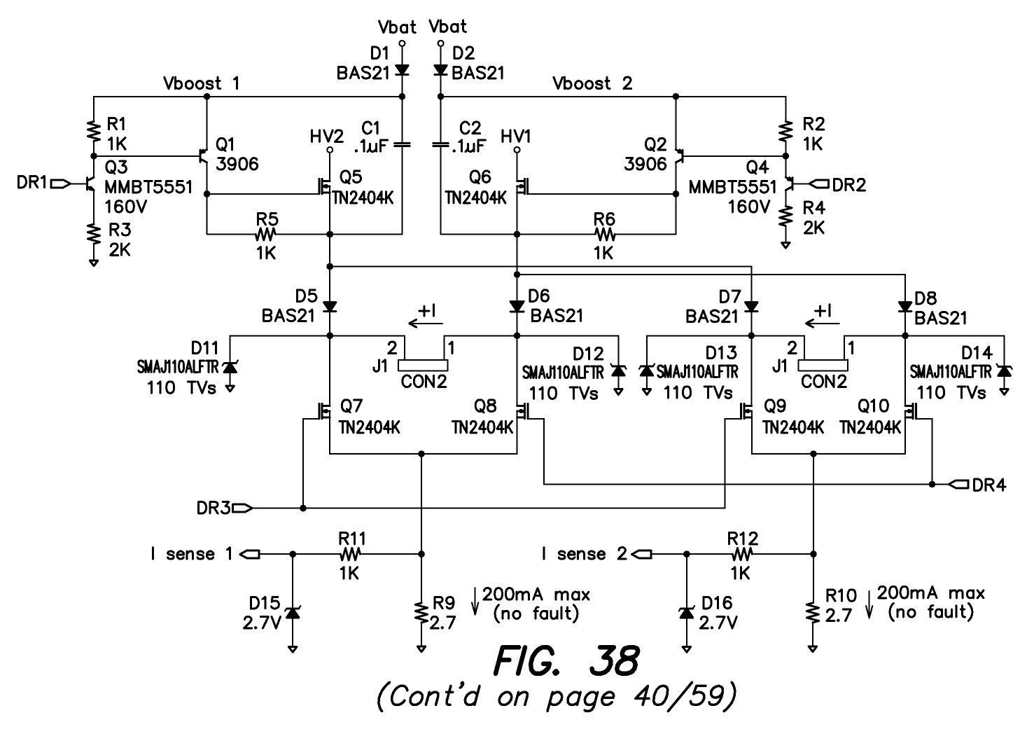

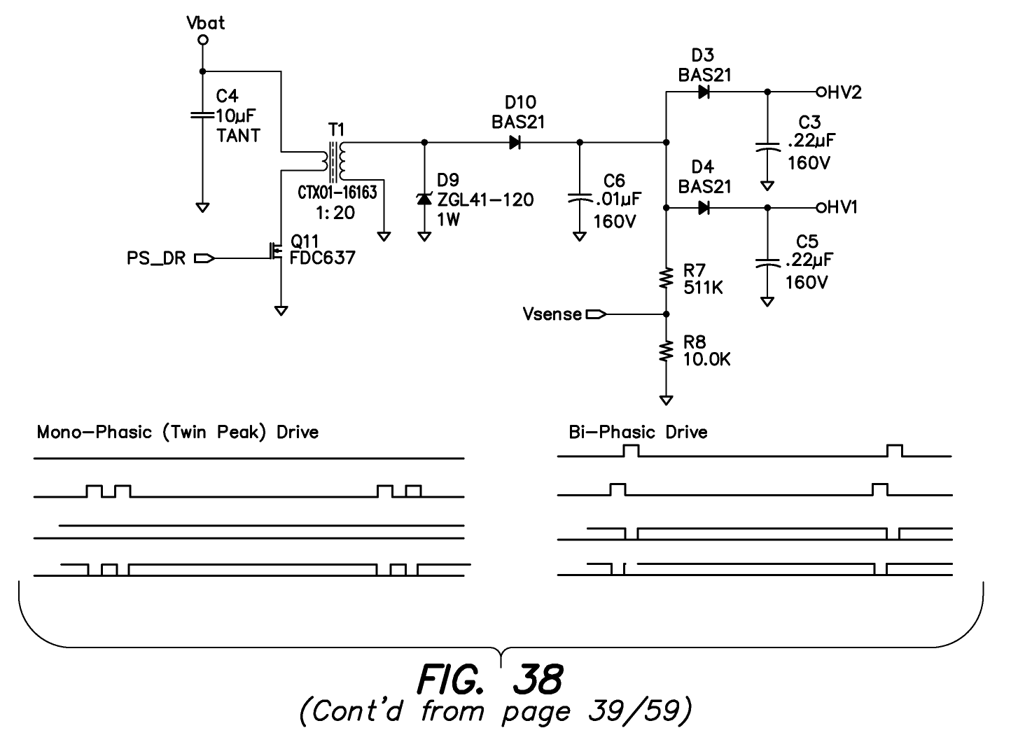

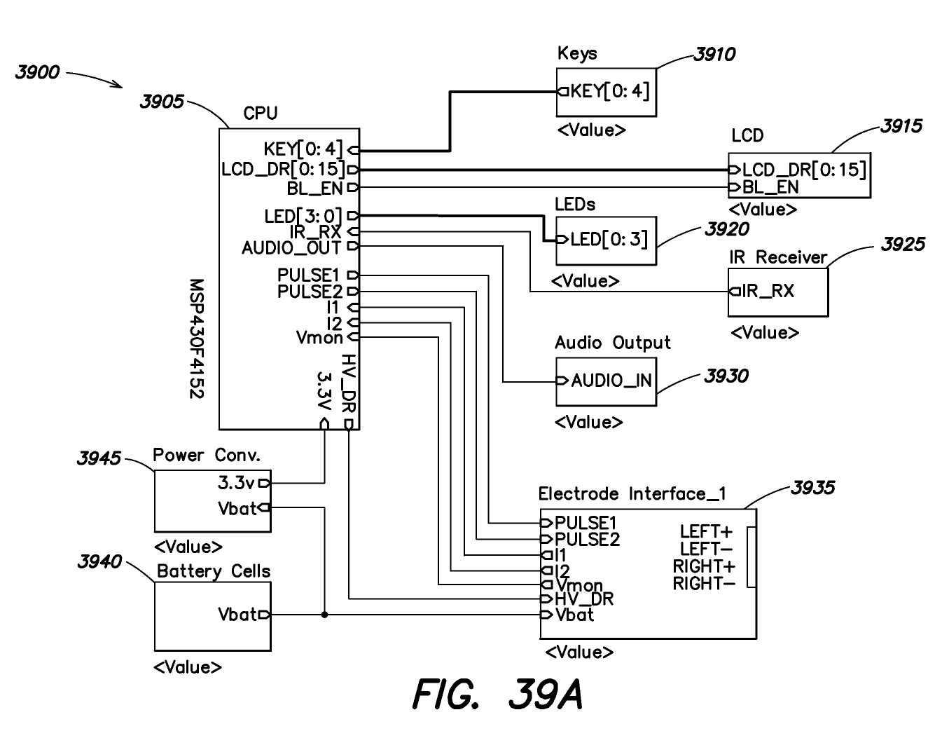

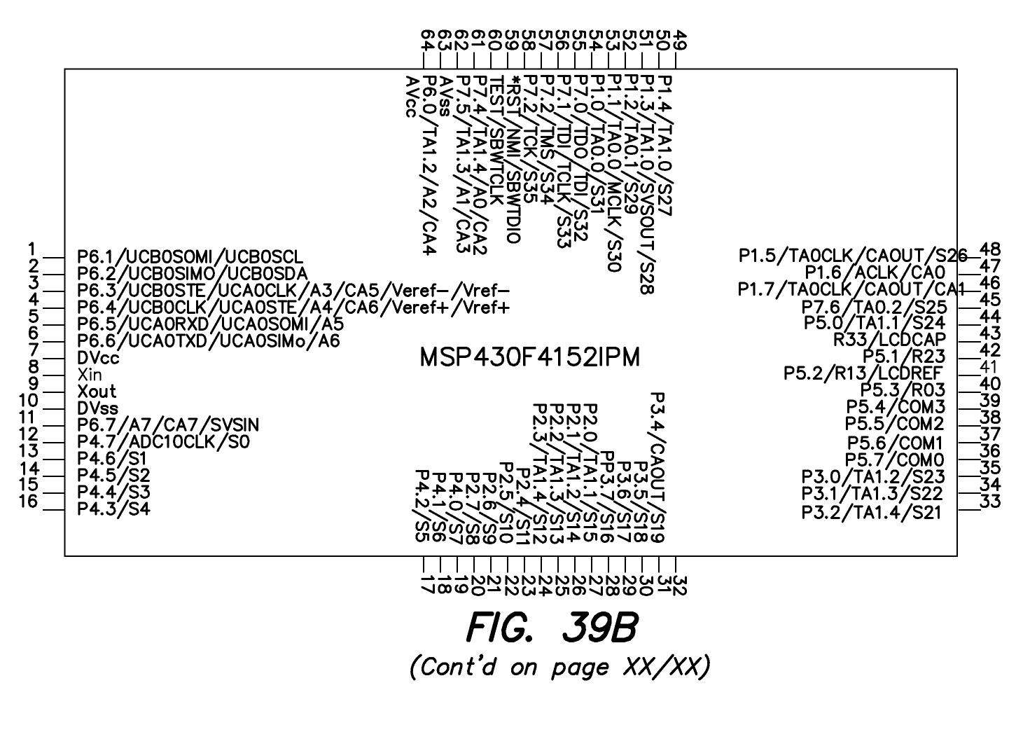

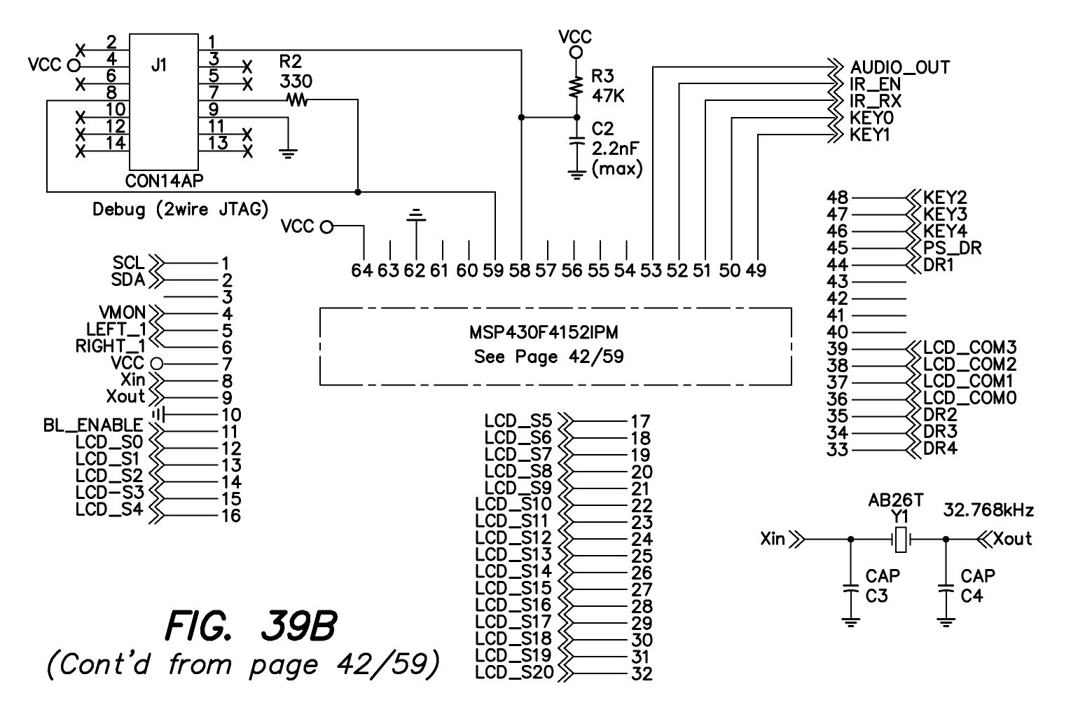

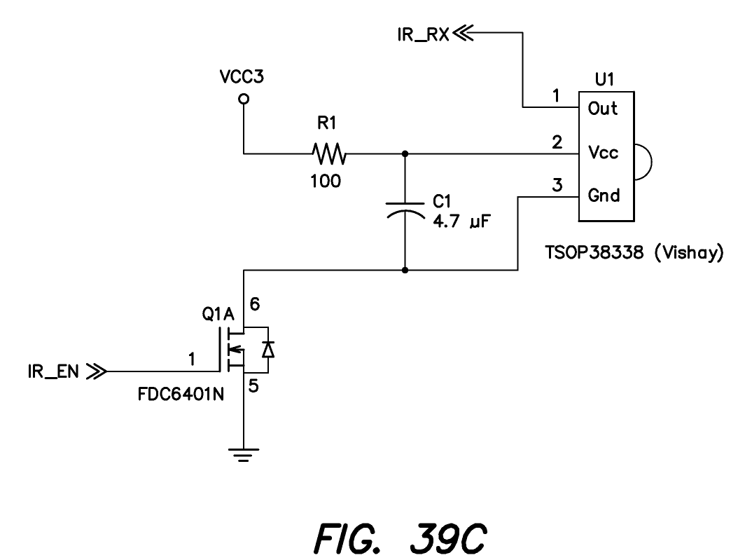

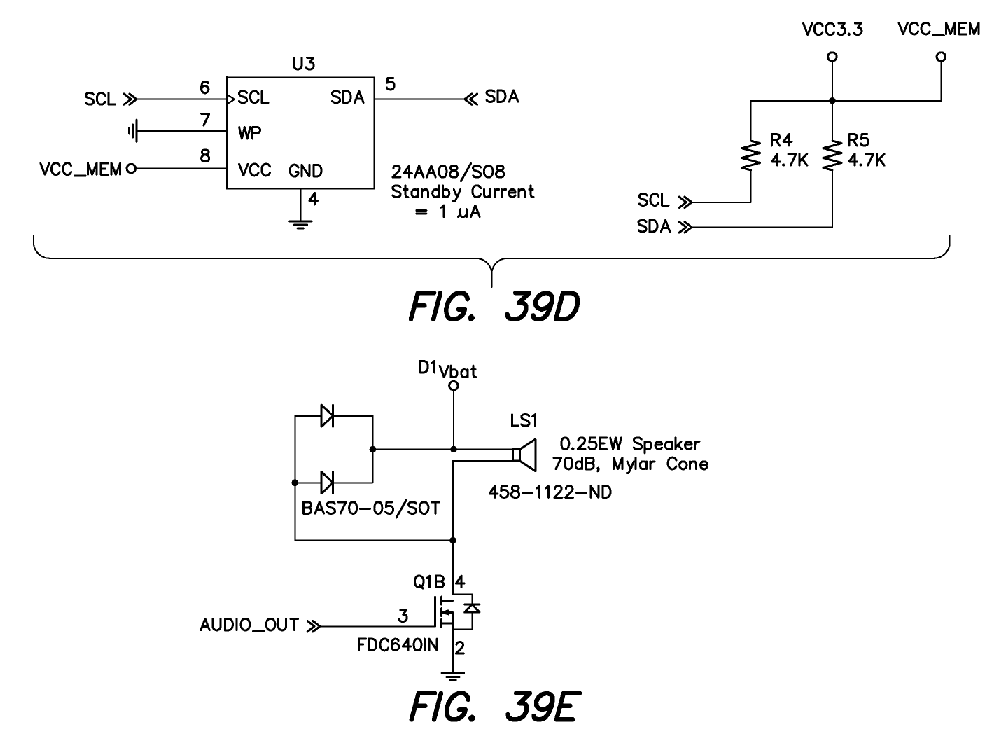

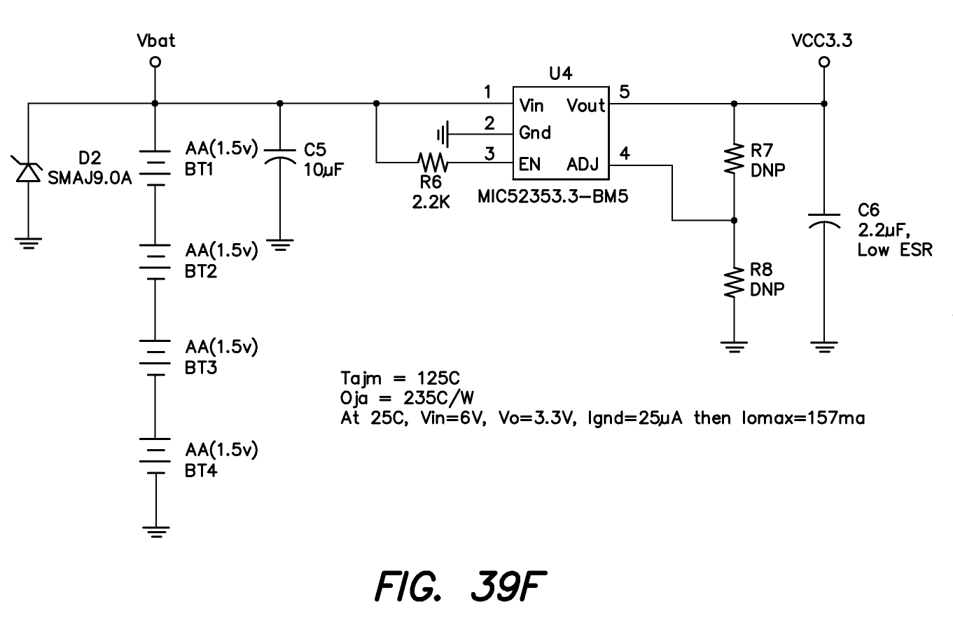

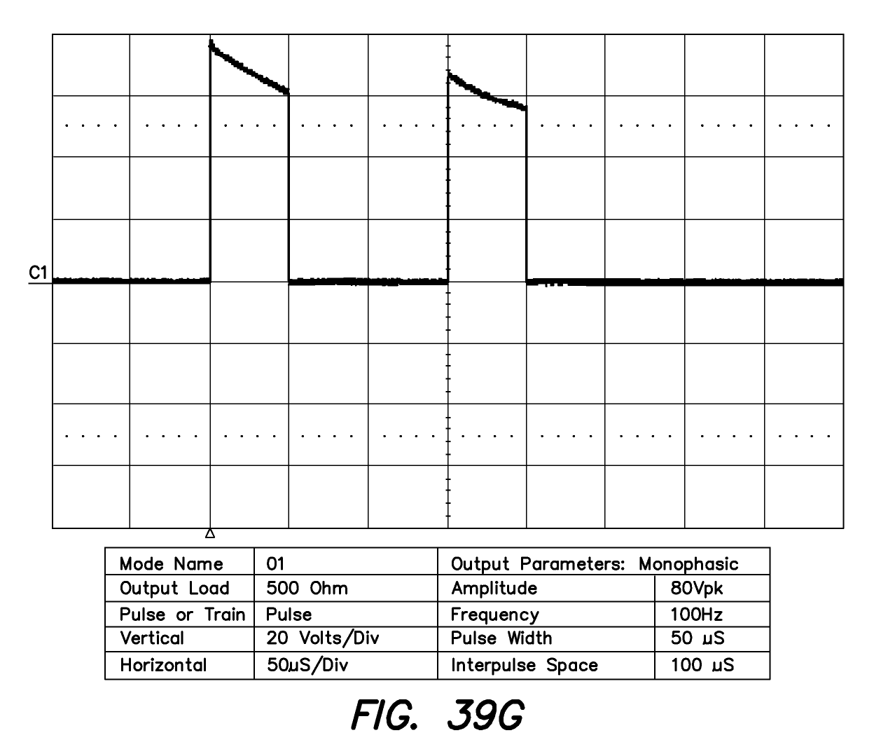

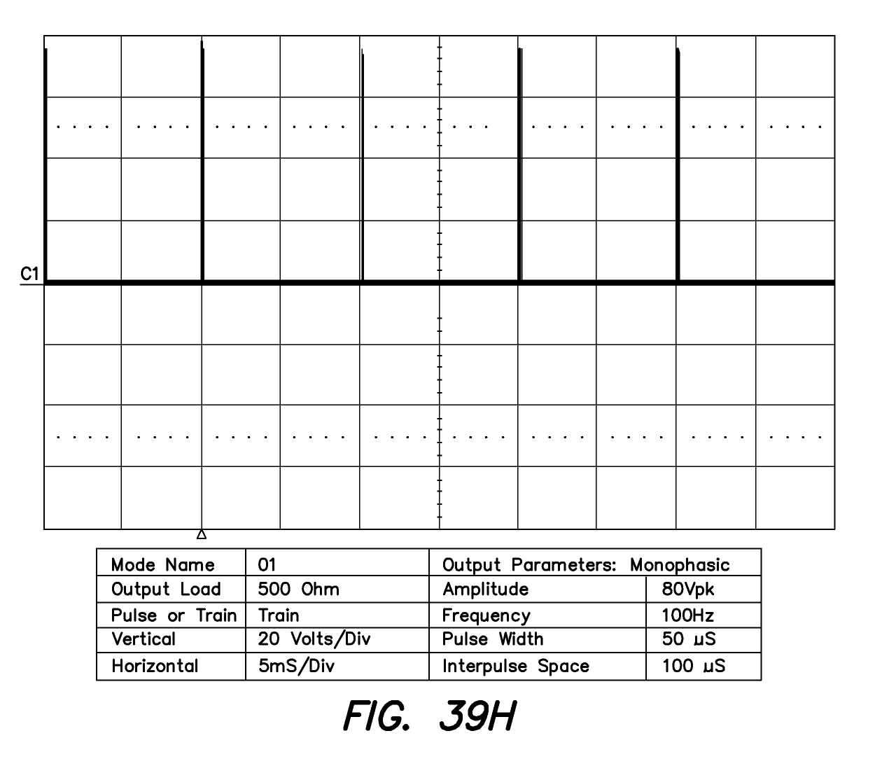

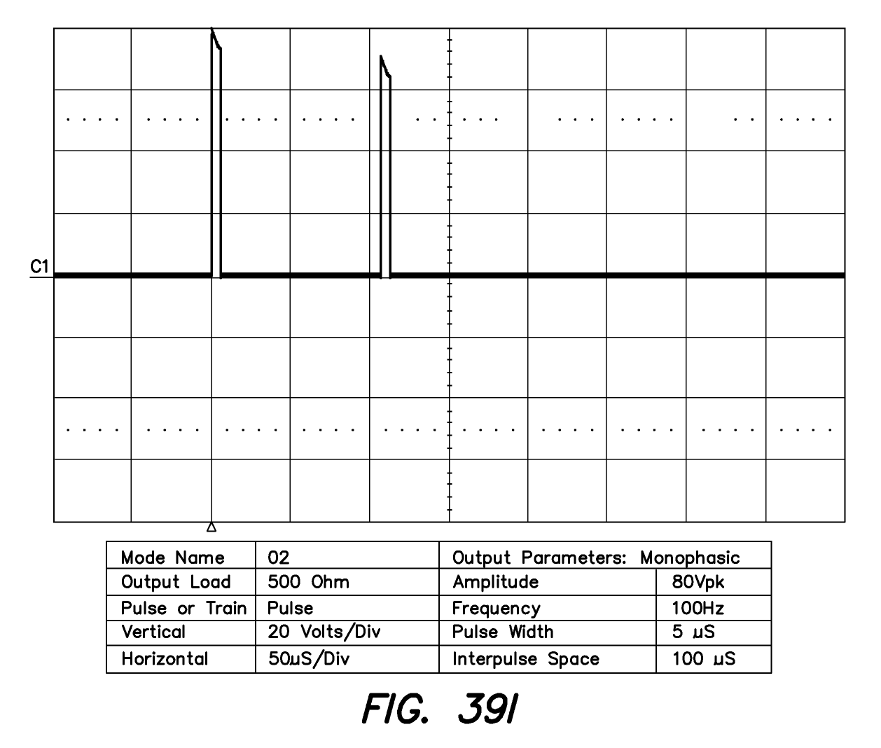

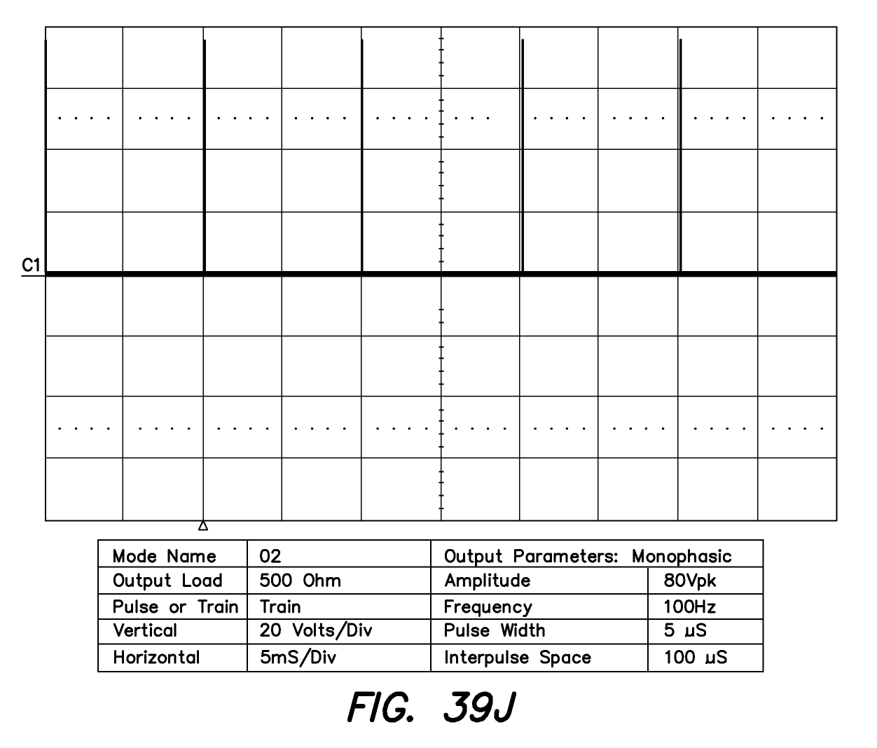

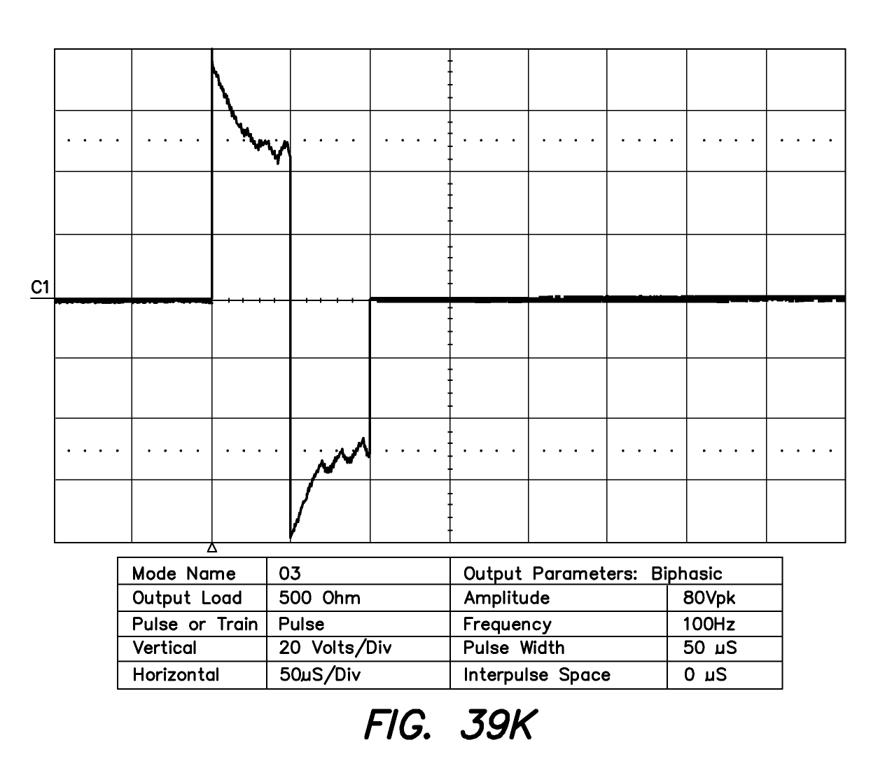

A pulse generator and associated circuitry for implementing the treatment methods described herein can be provided. FIG. 38 shows a circuit diagram of a generator suitable for use to generate a monophasic or biphasic waveform. A block diagram of a controller that can be used with the device described herein and to provide the treatment methods described herein is shown in FIG. 39A, with the circuit diagrams of the particular blocks shown in FIGS. 39B-39F.

With reference to FIG. 39A, the device comprises a microcontroller, 3905, a power source and regulator, 3940 and 3945, user interface components, 3910, 3915, 3920, and 3930, a remote control interface, 3925, and the boost converter and output drivers, 3935, detailed in FIG. 38. The batteries, 3940, and low voltage regulator, 3945, are detailed in FIG. 39F. The microcontroller, 3905, detailed in FIG. 39B, provides all user interface functions: monitoring a set of control keys, 3910; providing output to an LCD display, 3915; controlling output enable LEDs, 3920; and an output tone, 3930; detailed in FIG. 39C. Additional user interface functions include monitoring an IR remote control via an IR receiver, 3925; detailed in FIG. 39C. User interface functions include:
a) Power on and off
b) Selecting and displaying a treatment modality (e.g., a monophasic or biphasic waveform and/or current parameters)
c) Setting a treatment duration
d) Selecting treatment of a single foot or both feet
e) Starting the treatment period
f) Displaying the remaining treatment time
g) Adjusting and displaying the treatment intensity
h) Adjusting the treatment intensity through remote control
i) Stopping the treatment prematurely
j) Audible tones for adjustments and end of treatment
   In addition to the user interface functions the microcontroller, 3905, monitors and controls the boost converter and output drivers, 3935, through the following signal lines.
a) HV_DR provides a pulsed control signal to drive the boost converter. The pulse width and timing of this pulse will determine the bus voltage delivered to the output drivers. This signal is marked as PS_DR on FIG. 38.
b) Vmon monitors the bus voltage delivered to the output drivers. The microcontroller monitors this signal to determine the pulse characteristics of HV_DR. This signal is marked as Vsense on FIG. 38.
c) PULSE1 and PULSE2 provide control signals to the output drivers which determine polarity and pulse width of the output voltage applied to the electrode outputs, LEFT+, LEFT−, RIGHT+, and RIGHT−. These signals are four distinct signal lines and marked as DR1, DR2, DR3, and DR4 on FIG. 38.
d) I1 and I2 monitor the pulse current in each output channel This signal will allow the microcontroller to determine if the output load is within the desired range. These signals are labeled as Isense1 and Isense2 on FIG. 38.

The boost converter circuitry and output drivers are detailed in FIG. 38. This circuitry is monitored and controlled by the microcontroller firmware through the signals listed above. During treatment the peak amplitude of the output pulses is determined by the bus voltage generated by the boost converter. The boost converter will generate a bus voltage between 0 and 80 volts from the batteries, 3940. The main components of the boost converter are transistor Q11, transformer T1, output diodes D10, D3 and D4, and output capacitors C6, C3, and C5. These components form a simple flyback type boost converter. The transistor on time as determined by the pulse length and frequency of PS_DR will determine the energy stored in the transformer, T1 and transferred to the output capacitors during the transistor off time. The user intensity setting entered through the user interface along with the bus voltage fed back by Vsense will allow the microcontroller to calculate the correct pulse length and frequency to achieve the output voltage desired. The output drivers of an H-bridge arrangement, Q5-Q10, with the bottom half split to allow independent monitoring of the current in each channel. This H-bridge topology allows the forward and reverse pulses of the output waveform to be synthesized by switching sequences generated by the firmware and hardware within the microcontroller, 3905. The drive waveforms, DR1-DR4, are shown in FIG. 38 for both the monophasic and biphasic waveforms. Level translation circuitry is also provided. This is the electronics that translates the logic level signals from the microcontroller to signals referenced to the high voltage bus voltage to provide switching signals for the output transistors. This level translation circuit is designed such that the output transistors cannot remain in the on state for longer than a fixed period. This provides two levels of protection. Firstly it limits the maximum period that a pulse can be applied to the user in normal operation. Secondly, it provides a further level of protection against microprocessor failure, since the microprocessor may be expected to fail with its outputs in a frozen state. The two arms of the H-bridge are fed by two bus capacitors, C3 and C5 isolated by two diodes, D3 and D4. The waveform exhibits high rates of a change on its forward and trailing edges, and is substantially of square wave form except for the droop in the bus which is the result of partial discharge of the bus capacitor supplying the energy for the pulse. It should be noted that the trailing edge has a rapid descent to zero volts. This is achieved by turning on both bottom devices in the H-bridge arrangement during the off period as shown in the drive waveform plots.

Using the controller above, the following illustrative waveforms and current parameters can be used.

| | Pulse Pairs | Pulse Width | Interpulse Period |
|---|---|---|---|
| Program | per second | (μsec) | (μsec) |
| | Twin Pulse Waveform | | |
| 01 | 100 | 50 | 100 |
| 02 | 100 | 5 | 100 |
| | Biphasic Waveform | | |
| 03 | 100 | 50 | 0 |
| 04 | 5 | 100 | 0 |

Illustrative waveforms, pulses and pulse trains for programs 01-04 noted in the table immediately above are shown in FIGS. 39G-39N. Depending on the selected program, treatment of different disorders may be targeted. For example, monophasic waveforms can be used to prevent or treat loss of sensation, whereas biphasic waveforms can be used, for example, to treat pain.

### Example 12

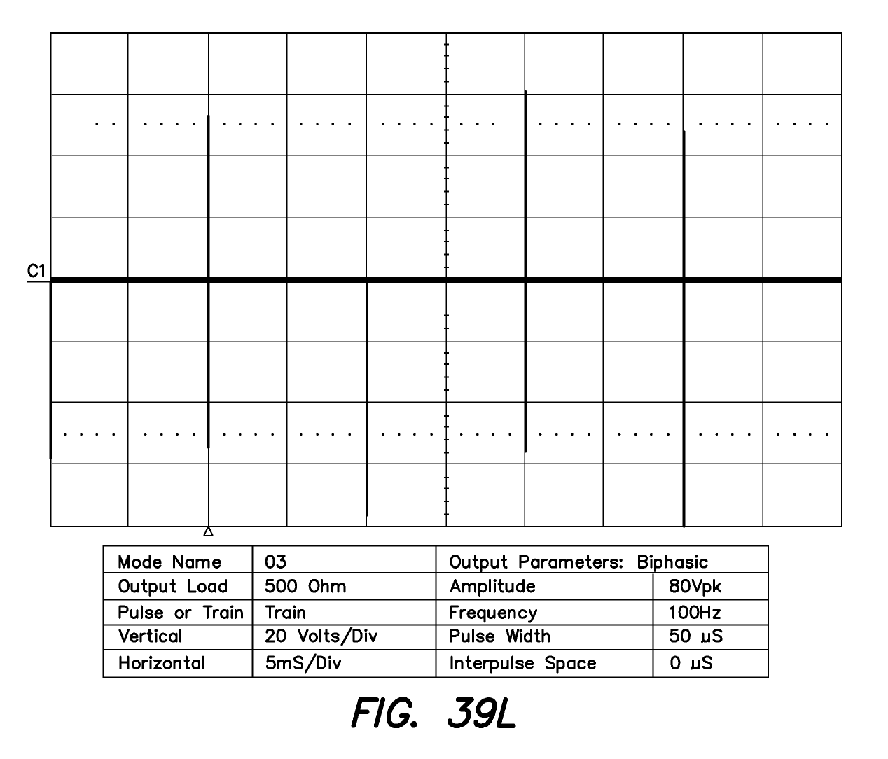

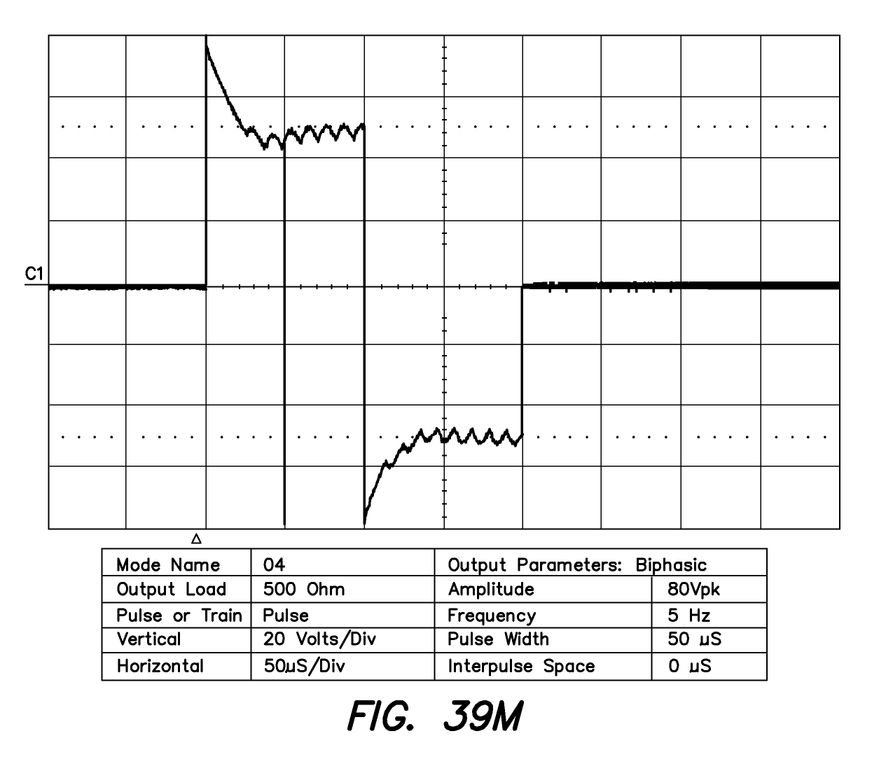

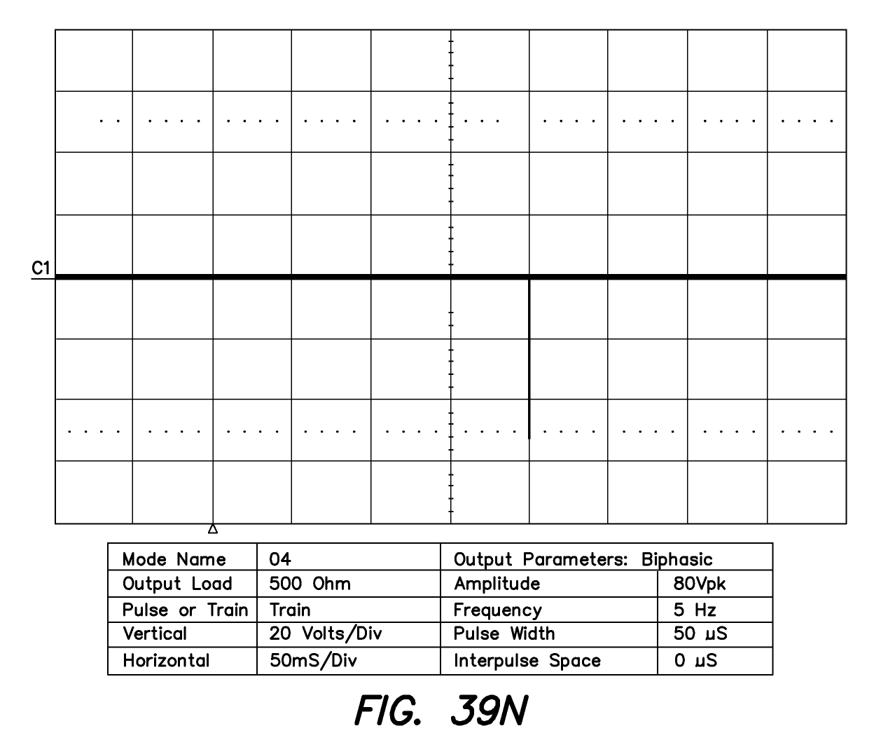

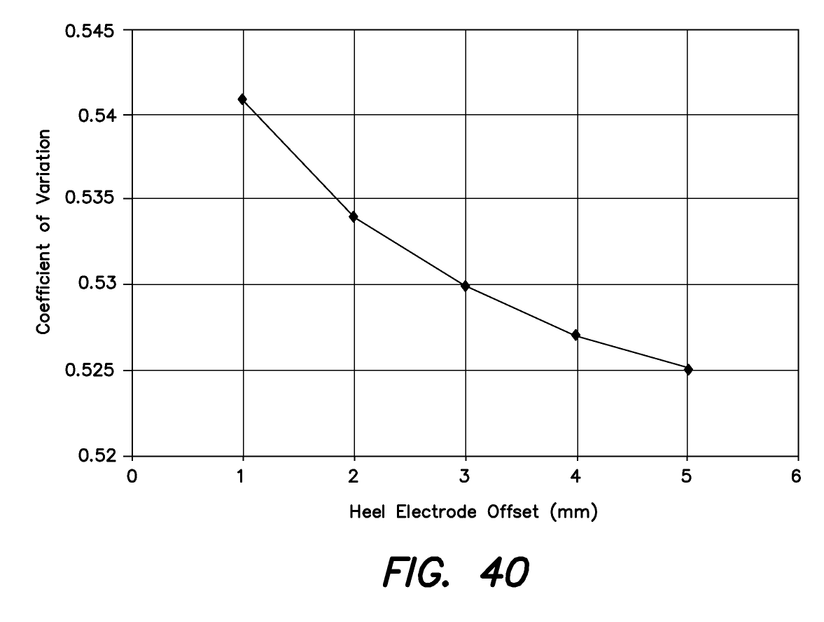

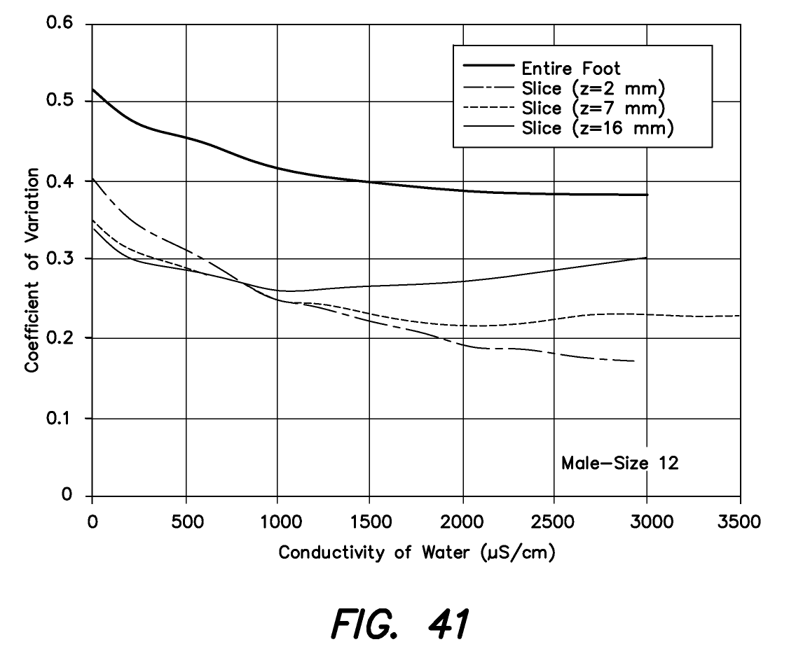

A finite element analysis was performed to determine the effects on uniformity of energy deposition of an electrode spacer and a range of electrical conductivity of the treatment solution. The graphs shown in FIGS. 40 and 41 were generated by a finite element analysis program that calculated the current flow within the water bath and foot. One metric for showing improvement in the design is the reduction of “hot spots” as shown by the coefficient of variation of the current density within the tissue of the foot.

FIG. 41 shows the relationship between the coefficient of variation and the conductivity of the water. In this chart the coefficient of variation is calculated across the volume of the foot and across three slices through the foot, at z=2 mm, 7 mm, and 16 mm, where z=0 mm is at the plantar surface of the foot. Evident in the z=2 mm plot there is a broad minima in the curve indicating a desired range of conductivity for the water. The z=2 mm plane is considered a desired region of interest for treating loss of sensation.

FIG. 40 shows the effect of spacing the heel electrode away from the foot. With the foot close to the electrode a high current area forms at the front edge of the electrode driving up the coefficient of variation. As the foot is spaced away from the electrode the relatively lower conductivity of the water forces the current to be spread out over a larger area. In this plot the coefficient of variation is calculated across the volume of the foot tissue.

### Example 13

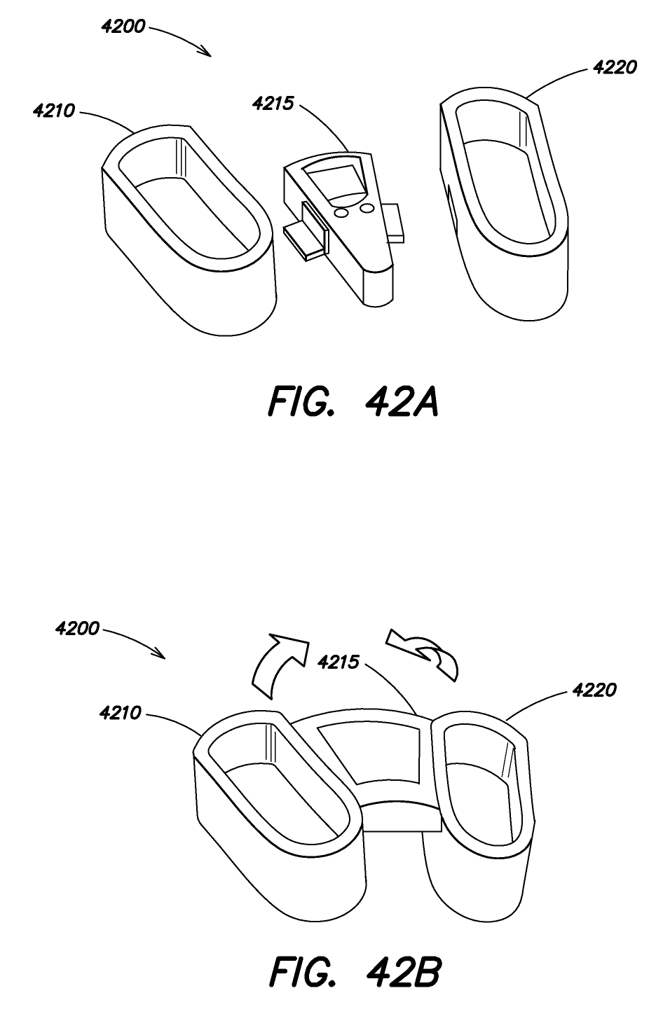

A treatment device that includes two separate compartments is shown in FIGS. 42A and 42B. The device 4200 includes a first compartment 4210 and a second compartment 4220, either of which may be configured as one or more of the compartments described herein. The compartments can be inserted into receptacles as described herein as shown in FIG. 42A, or the bridging controller can be inserted into or onto the compartments or a receptacle on the compartments as shown in FIG. 42B. A bridging controller 4215 can be used to select the desired treatment parameters. The controller 4215 contains all of the electronics, sets the appropriate spacing/positioning of the foot compartments and provides the electrical interfacing to the compartments. For storage the controller 4215 could store in one of the compartments and then the other compartments could stack on top of the other or could fold for storage.

### Example 14

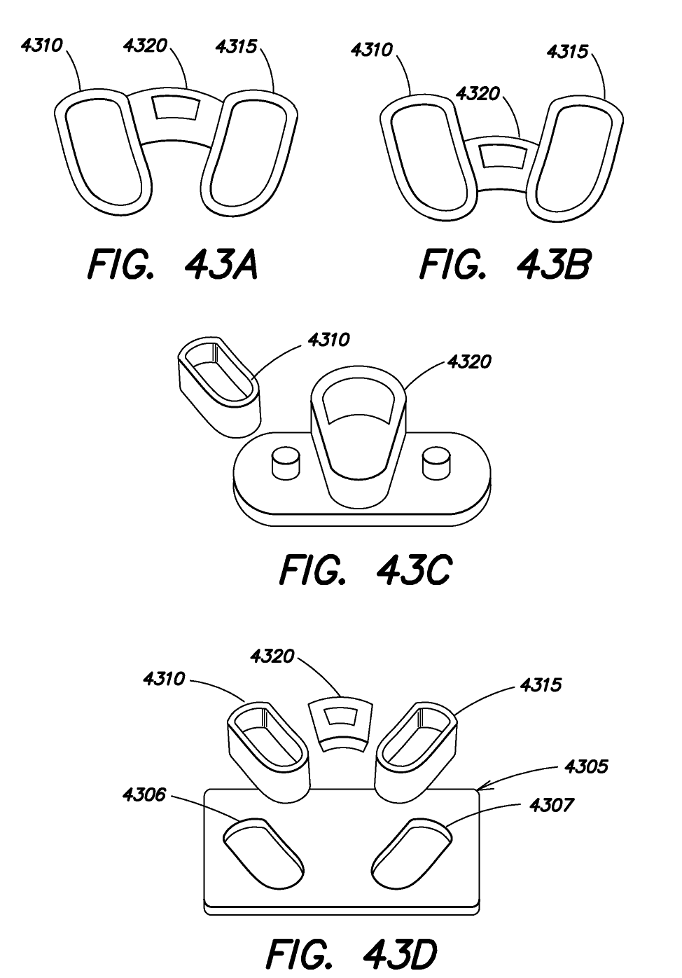

Treatment devices that illustrates a variation on the devices described in Example 13, which may include a mat that provides visual cues or mechanical cutouts which identify positioning of the compartments to set the correct spacing/positioning for treatment as well as allowing for easy placement of the bridge component interface onto or into connection points on the top surface of the compartments. Referring to FIGS. 43A, 43B, and 43D, a mat 4305 can be used to set the spacing between compartment 4310 and 4315. The mat includes depressions or areas 4306 and 4307 each of which is sized and arranged to receive one of the compartments 4310 and 4315. The depressions 4306 and 4307 can also be angled suitably such that the compartments 4310 and 4315 are angled once they are placed into the depressions 4306 and 4307. After or before placement of the compartments 4310 and 4315 into the depressions 4306 and 4307, respectively, of the mat 4305, a controller 4320 can be electrically coupled to the compartments 4310 and 4315 to permit treatment for loss of sensation using one or more of the methods described herein. As shown in FIGS. 43A and 43B, the exact position where the controller 4320 is electrically coupled to the compartments 4310 and 4315 can vary. In addition, the size and dimensions of the controller can also vary as shown in FIG. 43C. Further, FIG. 43C represents a configuration variation of the device of FIG. 42A where the compartments are placed onto receptacles attached to the controller 4320.

### Example 15

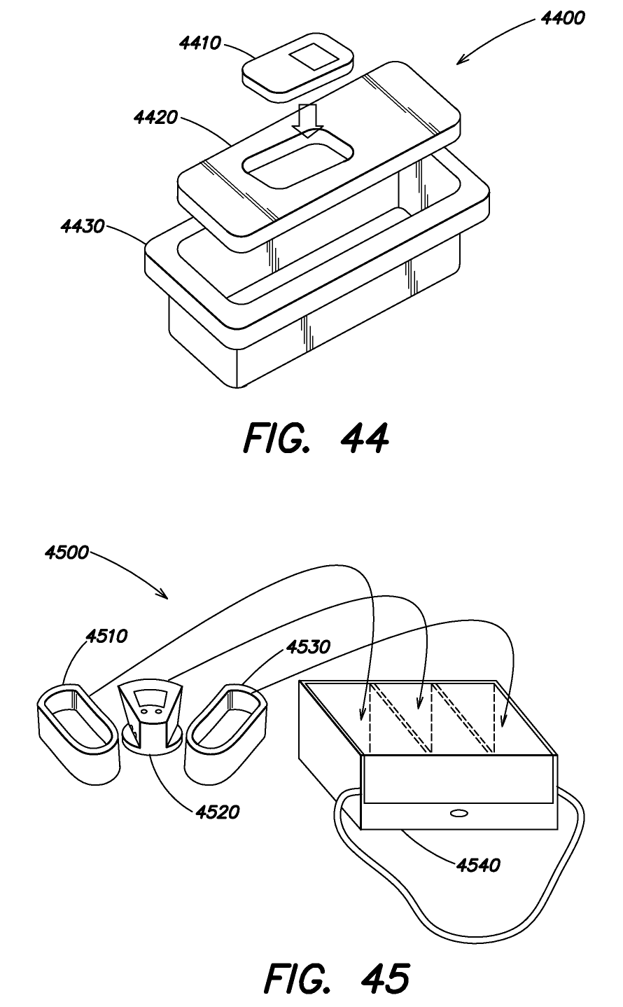

FIG. 44 is an illustration of a treatment device that includes a cover 4420, a controller 4410 configured to be inserted into the cover 4420 when not in use and a compartment 4430 configured to receive the cover 4420. The controller 4410 can be used to select a desired treatment method. The controller 4410 contains all of the electronics, is used with and placed into a mounting bridge (not shown) during treatment which sets the appropriate spacing/positioning of the foot compartments while also providing the electrical interfacing to the compartment 4430. In some examples, the compartment 4430 may be coupled to another compartment (not shown) through the mounting bridge such that treatment can be performed on both feet.

### Example 16

FIG. 45 is an illustration of a satchel or bag that can be used to transport a treatment device. The treatment device 4500 includes compartments 4510 and 4530 and a controller 4520. The components of the treatment device 4500 can be placed into a satchel 4540 having one or more sections or slots. For example, each compartment may be placed in its own section and the controller can be placed in another section.

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

What is claimed is:

1. A footbath comprising:
a housing;
a first and a second receptacle in the housing, each of the first and second receptacles sized and arranged to receive a compartment and comprising an electrical connector designed to electrically couple the compartment to the receptacle; and
a power source electrically coupled to the first and second receptacles, the power source configured to provide an effective amount of current to the compartment to prevent or treat loss of sensation.

2. The footbath of claim 1, in which the power source is configured to provide a waveform with an amplitude of 10-300 Volts.

3. The footbath of claim 1, in which the power source is configured to provide a pulsed current up to about 50 milliamperes, a pulse width between about 5 and about 100 microseconds, a pulse spacing from leading edge to leading edge of about 100 to 250 microseconds, and a pulse pair frequency between about 100 and about 200 Hertz.

4. The footbath of claim 1, in which the power source is configured to provide a monophasic waveform or a biphasic waveform.

5. The footbath of claim 1, in which each of the receptacles is independently movable to adjust the spacing between the receptacles.

6. The footbath of claim 1, further comprising a controller integrated into the housing and configured to receive user input.

7. The footbath of claim 1, in which the housing comprises a retaining mechanism configured to engage the compartment to retain the compartment in the receptacle.

8. The footbath of claim 1, further comprising a first electrode and a second electrode in each of the receptacles, the first electrode and the second electrode each configured to be electrically coupled to the compartment when the compartment is inserted into the receptacle.

9. The footbath of claim 8, in which the position of each of the first electrode and the second electrode is independently adjustable in each of the receptacles.

10. The footbath of claim 8, in which each receptacle further comprises a positioning mechanism to position the compartment in the receptacle.

11. A compartment sized and arranged to receive a human foot and comprising a housing, an electrical connector on the housing that is configured to electrically couple the compartment to a receptacle of a footbath, and a pair of electrodes in the housing.

12. The compartment of claim 11, further comprising a heel adjuster operative as a first electrode of the pair of electrodes and configured to be placed in contact with a heel of the human foot.

13. The compartment of claim 12, further comprising a conductive area adjacent to a toe portion of the compartment, the conductive area operative as a second electrode of the pair of electrodes.

14. The compartment of claim 11, further comprising a cover effective to provide a substantially fluid tight seal when in a closed position.

15. The compartment of claim 11, in which the surface of the compartment comprises a conductive material in areas that contact the human foot.

16. The compartment of claim 15, further comprising an insole in contact with the surface, the insole comprising a non-conductive substrate and removable areas effective to provide an electrical path between the conductive material and the foot at the removable areas.

17. The compartment of claim 15, further comprising an insole in contact with the surface, the insole comprising a conductive substrate and removable areas effective to act as insulators when present in the insole.

18. The compartment of claim 11, in which a depth of the compartment at a toe portion of the compartment is less than a depth of the compartment at a heel portion of the compartment to provide an angled compartment.

19. A kit comprising:
a footbath comprising a housing, at least one receptacle in the housing that is sized and arranged to receive a compartment, the receptacle comprising an electrical connector designed to electrically couple the compartment to the receptacle; and
a compartment comprising a connector configured to couple to the electrical connector of the receptacle to provide an electrical path between a pair of electrodes in the compartment and the receptacle.

20. The kit of claim 19, further comprising a power source configured to be electrically coupled to the receptacle, the power source further configured to be electrically coupled to a circuit in the footbath that is operative to provide an effective amount of current to a foot in the compartment for an effective duration to prevent or treat loss of sensation of the foot.

21. The kit of claim 19, further comprising a remote coupled to the housing and configured to receive user input.

22. The kit of claim 19, further comprising two receptacles in the housing, in which each of the receptacles is independently movable to adjust the spacing between the receptacles.

23. The kit of claim 19, in which the housing comprises a retaining mechanism configured to engage the compartment to retain the compartment in the receptacle.

24. A method comprising:
identifying and/or selecting a subject having loss of sensation in a foot; and
administering an effective amount of current to the foot to treat the sensation loss using the compartment of claim 11 and using a waveform with an amplitude of 10-300 Volts, a pulsed current up to about 50 milliamperes, a pulse width between about 5 and about 100 microseconds, pulse spacing from leading edge to leading edge of about 100 to 250 microseconds, a pulse pair frequency between about 100 and about 200 Hertz, a square pulse, and a monophasic or a biphasic waveform.

25. The method of claim 24, in which the administering step is performed in the absence of a drug agent.
